(19) United States
(12) Patent Application Publication
(10) Pub. No.: US 2007/0054262 A1
(43) Pub. Date: Mar. 8, 2007

(54) Methods of identifying optimal variants of peptide epitopes

(76) Inventors: Denise M. Baker, Poway, CA (US); Brian D. Livingston, San Diego, CA (US); Robert W. Chesnut, Cardiff-by-the-Sea, CA (US); Alessandro Sette, La Jolla, CA (US); Mark J. Newman, Carlsbad, CA (US)

Correspondence Address: STERNE, KESSLER, GOLDSTEIN & FOX, P.L.L.C., 1100 NEW YORK AVE., WASHINGTON, DC, 20005, US

(21) Appl. No.: 10/551,209

(22) PCT Filed: Mar. 29, 2004
(86) PCT No.: PCT/US04/09510

§ 371 Date: Sep. 28, 2005

Related U.S. Application Data
(60) Provisional application No. 60/458,026, filed on Mar. 28, 2003.

(51) Int. Cl. C12Q 1/70; G01N 33/569; G01N 33/554; G01N 33/53

(52) U.S. Cl. 435/5; 435/7.22; 435/7.31; 435/7.32

(57) ABSTRACT

The present invention is directed to methods for selecting a variant of a peptide epitope which induces a CTL response against another variant(s) of the peptide epitope, by determining whether the variant comprises only conserved residues, as defined herein, at non-anchor positions in comparison to the other variant(s). The present invention is also directed to variants identified by the methods above; peptides comprising such variants; nucleic acids encoding such variants and peptides; cells comprising such variants, and/or peptides, and/or nucleic acids; compositions comprising such variants, and/or peptides, and/or nucleic acids, and/or cells; as well as therapeutic and diagnostic methods for using such variants, peptides, nucleic acids, cells, and compositions.

### BACKGROUND OF THE INVENTION

1. Field of the Invention

This invention relates to the field of biology. In a particular embodiment, it relates to peptides, polynucleotides, and compositions useful to monitor or elicit an immune response to selected antigens, and methods of identifying such peptides and polynucleotides.

2. Related Art

HLA class I molecules are expressed on the surface of almost all nucleated cells. Following intracellular processing of antigens, epitopes from the antigens are presented as a complex with the HLA class I molecules on the surface of such cells. CTL recognize the peptide-HLA class I complex, which then results in the destruction of the cell bearing the HLA-peptide complex directly by the CTL and/or via the activation of non-destructive mechanisms e.g., the production of interferon, that inhibit viral replication.

Human Immunodeficiency Virus. Acquired immunodeficiency syndrome (AIDS) caused by infection with human immunodeficiency virus-1 (HIV-1) represents a major world health problem. Estimates indicate that about 16,000 people worldwide are infected with HIV each day.

The development of anti-viral drugs has been a major advancement in reducing viral loads in HIV infected patients. Highly active retroviral therapy (HAART) has been shown to reduce viremia to nearly undetectable levels. However, current drug therapies are not practicable as a long term solution to the HIV epidemic. HAART therapy is severely limited due to poor tolerance for the drugs and the emergence of drug-resistant virus. Moreover, replication competent HIV persists in the lymphoid tissue of patients who have responded to HAART, thus serving as a reservoir of virus. Lastly, current anti-retroviral drug therapies have little impact upon the global epidemic: almost 90% of the world's HIV infected population resides within countries lacking financial resources for these drugs. Thus, a need exists for an efficacious vaccine to both prevent and treat HIV infection.

Virus-specific, human leukocyte antigen (HLA) class I-restricted cytotoxic T lymphocytes (CTL) are known to play a major role in the prevention and clearance of virus infections in vivo (Oldstone et al., Nature 321:239, 1989; Jamieson et al., J. Virol. 61:3930, 1987; Yap et al, Nature 273:238, 1978; Lukacher et al., J. Exp. Med. 160:814, 1994; McMichael et al., N. Engl. J. Med. 309:13, 1983; Sethi et al., J. Gen. Virol. 64:443, 1983; Watari et al., J. Exp. Med. 165:459, 1987; Yasukawa et al., J. Immunol. 143:2051, 1989; Tigges et al., J. Virol. 66:1622, 1993; Reddenhase et al., J. Virol. 55:263, 1985; Quinnan et al., N. Engl. J. Med. 307:6, 1982).

While immune correlates of protective immunity against HIV infection are not well defined, there is a growing body of evidence that suggests CTL are important in controlling HIV infection. HIV-specific CTL responses can be detected early in infection and the appearance of the responses corresponds to the time in infection at which initial viremia is reduced (Pantaleo et al., Nature 370:463, 1994; Walker et al., Proc. Natl. Acad. Sci. 86:9514, 1989). In addition, HIV replication in infected lymphocytes can be inhibited by incubation with autologous CTL (see, e.g., Tsubota et al., J. Exp. Med. 169:1421, 1989). These data are supported by recent studies that indicate CTL are required for controlling viral replication in a SIV/rhesus animal model (Schmitz et al., Science 283:857, 1999), and additionally supported by studies that demonstrate that CTL exert selective pressure on HIV populations as evidenced by the eventual predominance of viruses with amino acid replacements in those regions of the virus to which CTL responses are directed (see, e.g., Borrow et al., Nature Med. 3:205-211, 1997; Price et al., Proc. Nat. Acad. Sci. 94:12890-1895, 1997; Koenig et al., Nature Med. 1:330-336, 1995; and Haas et al., J. Immunol. 157:4212-4221, 1996).

Virus-specific T helper lymphocytes are also known to be critical for maintaining effective immunity in chronic viral infections. Historically, HTL responses were viewed as primarily supporting the expansion of specific CTL and B cell populations; however, more recent data indicate that HTL may directly contribute to the control of virus replication. For example, a decline in CD4⁺ T cells and a corresponding loss in HTL function characterize infection with HIV (Lane et al., New Engl. J. Med. 313:79, 1985). Furthermore, studies in HIV infected patients have also shown that there is an inverse relationship between virus-specific HTL responses and viral load, suggesting that HTL play a role in viremia (see, e.g., Rosenberg et al., Science 278:1447, 1997).

A fundamental challenge in the development of an efficacious HIV vaccine is the heterogeneity observed in HIV. The virus, like some other infectious agents including retroviruses, rapidly mutates during replication resulting in the generation of virus that can escape anti-viral therapy and immune recognition (Borrow et al., Nature Med. 3:205, 1997). In addition, HIV can be classified into a variety of subtypes that exhibit significant sequence divergence (see, e.g., Lukashov et al., AIDS 12:S43, 1998). In view of the heterogeneous nature of HIV, and the heterogeneous immune response observed with HIV infection, induction of a multi-specific cellular immune response directed simultaneously against multiple HIV epitopes appears to be important for the development of an efficacious vaccine against HIV. There is a need to establish such vaccine embodiments which elicit immune responses of sufficient breadth and vigor to prevent and/or clear HIV infection.

Hepatitis B Virus. Chronic infection by hepatitis B virus (HBV) affects at least 5% of the world's population and is a major cause of cirrhosis and hepatocellular carcinoma (Hoofnagle, J., N. Engl. J. Med. 323:337, 1990; Fields, B. and Knipe, D., In: Fields Virology 2:2137, 1990). The World Health Organization lists hepatitis B as a leading cause of death worldwide, close behind chronic pulmonary disease, and more prevalent than AIDS. Chronic HBV infection can range from an asymptomatic carrier state to continuous hepatocellular necrosis and inflammation, and can lead to hepatocellular carcinoma.

The immune response to HBV is believed to play an important role in controlling hepatitis B infection. A variety of humoral and cellular responses to different regions of the HBV nucleocapsid core and surface antigens have been identified. T cell mediated immunity, particularly involving class I human leukocyte antigen-restricted cytotoxic T lymphocytes (CTL), is believed to be crucial in combatting established HBV infection.

Several studies have emphasized the association between self-limiting acute hepatitis and multispecific CTL responses (Penna, A. et al., J. Exp. Med. 174:1565, 1991; Nayersina, R. et al., J. Immunol. 150:4659, 1993). Spontaneous and interferon-related clearance of chronic HBV infection is also associated with the resurgence of a vigorous CTL response (Guidotti, L. G. et al., Proc. Natl. Acad. Sci. USA 91:3764, 1994). In all such cases the CTL responses are polyclonal, and specific for multiple viral proteins including the HBV envelope, core and polymerase antigens. By contrast, in patients with chronic hepatitis, the CTL activity is usually absent or weak, and antigenically restricted.

The crucial role of CTL in resolution of HBV infection has been further underscored by studies using HBV transgenic mice. Adoptive transfer of HBV-specific CTL into mice transgenic for the HBV genome resulted in suppression of virus replication. This effect was primarily mediated by a non-lytic, lymphokine-based mechanism (Guidotti, L. G. et al., Proc. Natl. Acad. Sci. USA 91:3764, 1994; Guidotti, L. G., Guilhot, S., and Chisari, F. V. J. Virol. 68:1265, 1994; Guidotti, L. G. et al., J. Virol. 69:6158, 1995; Gilles, P. N., Fey, G., and Chisari, F. V., J. Virol. 66:3955, 1992).

As is the case for HLA class I restricted responses, HLA class II restricted T cell responses are usually detected in patients with acute hepatitis, and are absent or weak in patients with chronic infection (Chisari, F. V. and Ferrari, C., Annu. Rev. Immunol. 13:29, 1995). HLA Class II responses are tied to activation of helper T cells (HTLs) Helper T lymphocytes, which recognize Class II HLA molecules, may directly contribute to the clearance of HBV infection through the secretion of cytokines which suppress viral replication (Franco, A. et al., J. Immunol. 159:2001, 1997). However, their primary role in disease resolution is believed to be mediated by inducing activation and expansion of virus-specific CTL and B cells.

In view of the heterogeneous immune response observed with HBV infection, induction of a multi-specific cellular immune response directed simultaneously against multiple epitopes appears to be important for the development of an efficacious vaccine against HBV. There is a need to establish vaccine embodiments that elicit immune responses that correspond to responses seen in patients that clear HBV infection. Epitope-based vaccines appear useful.

Hepatitis C Virus. Hepatitis C virus (HCV) infection is a global human health problem with approximately 150,000 new reported cases each year in the U.S. alone. HCV is a single stranded RNA virus, and is the etiological agent identified in most cases of non-A, non-B post-transfusion and post-transplant hepatitis, and is a common cause of acute sporadic hepatitis (Choo et al., Science 244:359, 1989; Kuo et al., Science 244:362, 1989; and Alter et al., in: Current Perspective in Hepatology, p. 83, 1989). It is estimated that more than 50% of patients infected with HCV become chronically infected and, of those, 20% develop cirrhosis of the liver within 20 years (Davis et al., New Engl. J. Med. 321:1501, 1989; Alter et al., in: Current Perspective in Hepatology, p. 83, 1989; Alter et al., New Engl. J. Med. 327:1899, 1992; and Dienstag, J. L. Gastroenterology 85:430, 1983). Moreover, the only therapy available for treatment of HCV infection is interferon-α. Most patients are unresponsive, however, and among the responders, there is a high recurrence rate within 6-12 months of cessation of treatment (Liang et al., J. Med. Virol. 40:69, 1993). Ribaviron, a guanosine analog with a broad spectrum activity against many RNA and DNA viruses, has been shown in clinical trials to be effective against chronic HCV infection when used in combination with interferon-α (see, e.g. Poynard et al., Lancet 352:1426-1432, 1998; Reichard et al., Lancet 351:83-87, 1998) However, the response rate is still well below 50%.

Virus-specific, human leukocyte antigen (HLA) class I-restricted cytotoxic T lymphocytes (CTL) are known to play a major role in the prevention and clearance of virus infections in vivo (Oldstone et al., Nature 321:239, 1989; Jamieson et al., J. Virol. 61:3930, 1987; Yap et al, Nature 273:238, 1978; Lukacher et al., J. Exp. Med. 160:814, 1994; McMichael et al., N. Engl. J. Med. 309:13, 1983; Sethi et al., J. Gen. Virol. 64:443, 1983; Watari et al., J. Exp. Med. 165:459, 1987; Yasukawa et al., J. Immunol. 143:2051, 1989; Tigges et al., J. Virol. 66:1622, 1993; Reddenhase et al., J. Virol. 55:263, 1985; Quinnan et al., N. Engl. J. Med. 307:6, 1982).

In view of the heterogeneous immune response observed with HCV infection, induction of a multi-specific cellular immune response directed simultaneously against multiple HCV epitopes appears to be important for the development of an efficacious vaccine against HCV. There is a need, however, to establish vaccine embodiments that elicit immune responses that correspond to responses seen in patients that clear HCV infection.

Human Papillomavirus. Human papillomavirus (HPV) is a member of the papillomaviridae, a group of small DNA viruses that infect a variety of higher vertebrates. More than 80 types of HPVs have been identified. Of these, more than 30 can infect the genital tract. Some types, generally types 6 and 11, may cause genital warts, which are typically benign and rarely develop into cancer. Other strains of HPV, “cancer-associated”, or “high-risk” types, can more frequently lead to the development of cancer. The primary mode of transmission of these strains of HPV is through sexual contact.

The main manifestations of the genital warts are cauliflower-like condylomata acuminata that usually involve moist surfaces; keratotic and smooth papular warts, usually on dry surfaces; and subclinical “flat” warts, which are found on any mucosal or cutaneous surface (Handsfield, H., Am. J. Med. 102(5A):16-20, 1997). These warts are typically benign but are a source of inter-individual spread of the virus (Ponten, J. & Guo, Z., Cancer Surv. 32:201-29, 1998). At least three HPV strains associated with genital warts have been identified: type 6a (see, e.g., Hofmann, K. J., et al., Virology 209(2):506-518, 1995), type 6b (see, e.g., Hofmann et al., supra) and type 11 (see, e.g., Dartmann, K. et al., Virology 151(1):124-130, 1986).

Cancer-associated HPVs have been linked with cancer in both men and women; they include, but are not limited to, HPV-16, HPV-18, HPV-31, HPV-45, HPV-33 and HPV-56. Other HPV strains, including types 6 and 11 as well as others, e.g., HPV-5 and HPV-8, are less frequently associated with cancer. The high risk types are typically associated with the development of cervical carcinoma and premalignant lesions of the cervix in women, but are also associated with similar malignant and premalignant lesions at other anatomic sites within the lower genital or anogenital tract. These lesions include neoplasia of the vagina, vulva, perineum, the penis, and the anus. HPV infection has also been associated with respiratory tract papillomas, and rarely, cancer, as well as abnormal growth or neoplasia in other epithelial tissues. See, e.g. VIROLOGY, 2^{ND} ED, Fields et al., Eds. Raven Press, New York, 1990, Chapters 58 and 59, for a review of HPV association with cancer.

The HPV genome consists of three functional regions, the early region, the late region, and the “long control region”. The early region gene products control viral replication, transcription and cellular transformation They include the HPV E1 and E2 proteins, which play a role in HPV DNA replication, and the E6 and E7 oncoproteins, which are involved in the control of cellular proliferation. The late region include the genes that encode the structural proteins L1 and L2, which are the major and minor capsid proteins, respectively. The “long control region” contains such sequences as enhancer and promoter regulatory regions.

HPV expresses different proteins at different stages of the infection, for example early, as well as late, proteins. Even in latent infections, however, early proteins are often expressed and are therefore useful targets for vaccine-based therapies. For example, high-grade dysplasia and cervical squamous cell carcinoma continue to express E6 and E7, which therefore can be targeted to treat disease at both early and late stages of infection.

Treatment for HPV infection is often unsatisfactory because of persistence of virus after treatment and recurrence of clinically apparent disease is common. The treatment may require frequent visits to clinics and is not directed at elimination of the virus but at clearing warts. Because of persistence of virus after treatment, recurrence of clinically apparent disease is common.

Thus, a need exists for an efficacious vaccine to both prevent and treat HPV infection and to treat cancer that is associated with HPV infection. Effective HPV vaccines would be a significant advance in the control of sexually transmissable infections and could also protect against clinical disease, particularly cancers such as cervical cancer. (see, e.g., Rowen, P. & Lacey, C., Dermatologic Clinics 16(4):835-838, 1998).

Virus-specific, human leukocyte antigen (HLA) class I-restricted cytotoxic T lymphocytes (CTL) are known to play a major role in the prevention and clearance of virus infections in vivo (Oldstone et al., Nature 321:239, 1989; Jamieson et al., J. Virol. 61:3930, 1987; Yap et al, Nature 273:238, 1978; Lukacher et al., J. Exp. Med. 160:814, 1994; McMichael et al., N. Engl. J. Med. 309:13, 1983; Sethi et al., J. Gen. Virol. 64:443, 1983; Watari et al., J. Exp. Med. 165:459, 1987; Yasukawa et al., J. Immunol. 143:2051, 1989; Tigges et al., J. Virol. 66:1622, 1993; Reddenhase et al., J. Virol. 55:263, 1985; Quinnan et al., N. Engl. J. Med. 307:6, 1982).

Virus-specific T helper lymphocytes are also known to be critical for maintaining effective immunity in chronic viral infections. Historically, HTL responses were viewed as primarily supporting the expansion of specific CTL and B cell populations; however, more recent data indicate that HTL may directly contribute to the control of virus replication. For example, a decline in CD4⁺ T cells and a corresponding loss in HTL function characterize infection with HIV (Lane et al., New Engl. J. Med. 313:79, 1985). Furthermore, studies in HIV infected patients have also shown that there is an inverse relationship between virus-specific HTL responses and viral load, suggesting that HTL plays a role in viremia (see, e.g., Rosenberg et al., Science 278:1447, 1997).

The development of vaccines with prophylactic and therapeutic efficacy against HPV is ongoing. Early vaccine development was hampered by the inability to culture HPV. With the introduction of cloning techniques and protein expression, however, some attempts have been made to stimulate humoral and CTL response to HPV (See, e.g., Rowen, P. & Lacey, C., Dermatologic Clinics 16(4):835-838 (1998)). Studies to date, however, have been inconclusive.

Activation of T helper cells and cytotoxic lymphocytes (CTLs) in the development of vaccines has also been analyzed. Lehtinen, M., et al. for instance, has shown that some peptides from the E2 protein of HPV type 16 activate T helper cells and CTLs (Biochem. Biophys. Res. Commun. 209(2):541-6 (1995). Similarly, Tarpey et al, has shown that some peptides from HPV type 11 E7 protein can stimulate human HPV-specific CTLs in vitro (Immunology 81:222-227 (1994)) and Borysiewicz et al. have reported a recombinant vaccinia virus expressing HPV 16 and HPV 17 E6 and E7 that stimulated CTL responses in at least one patient (Lancet 347:1347-1357, 1996).

Plasmodium falciparum and Malaria. Malaria, which is caused by infection with the parasite Plasmodium falciparum (PF), represents a major world health problem. Approximately 500 million people in the world are at risk from the disease, with approximately 200 million people actually harboring the parasites. An estimated 1 to 2 million deaths occur each year due to malaria. (Miller et al., Science 234:1349, 1986).

Fatal outcomes are not confined to first infections, and constant exposure is apparently a prerequisite for maintaining immunity. Naturally acquired sterile immunity is rare, if it exists at all. Accordingly, major efforts to develop an efficacious malaria vaccine have been undertaken.

Human volunteers injected with irradiated PF sporozoites are resistant to subsequent sporozoite challenges, which demonstrates that development of a malaria vaccine is indeed immunologically feasible. Furthermore, these immune individuals developed a vigorous response, including antibodies, and cytotoxic T lymphocyte (CTL) and helper T lymphocyte (HTL) components, directed against multiple antigens. Reproducing the breadth and multiplicity of this response in a vaccine, however, is a task of large proportions. The epitope approach, as described herein, may represent a solution to this challenge, in that it allows the incorporation of various antibody, CTL and HTL epitopes, from various proteins, in a single vaccine composition.

Anti-sporozoite antibodies are by themselves, in general, not completely efficacious in clearing the infection (Egan et al., Science 236:453, 1987). However, high concentrations of antibodies directed against the repeated region of the major B cell antigen of the sporozoite/circumsporozoite protein (CSP) have been shown to prevent liver cell infection in certain experimental models (Egan et al., Science 236:453, 1987; Potocnjak, P. et al., Science 207:71, 1980). The present inventors have shown that constructs encompassing CSP-repeat B cell epitopes and the optimized helper epitope PADRE™ (San Diego, Calif.) are highly immunogenic, and can protect in vitro against sporozoite invasion in both mouse and human liver cells, and protect mice in vivo against live sporozoite challenge (Franke et al., Vaccine 17:1201-1205, 1999)

PF-specific CD4⁺ T cells also have a role in malarial immunity beyond providing help for B cell and CTL responses. Experiments by Renia et al. (Renia, et al., Proc. Natl. Acad. Sci. USA 88:7963, 1991) demonstrated that HTLs directed against the Plasmodium yoelli CS protein could in fact adoptivley transfer protection against malaria.

Considerable data implicate CTLs in protection against pre-erythrocytic-stage malaria. CD8⁺ CTLs can eliminate Plasmodium berghei- or Plasmodium yoelii-infected mouse hepatocytes from in vitro culture in a major histocompatibility complex (MHC)-restricted and antigen-restricted manner (Hoffman et al,, Science 244:1078-1081, 1989; Weiss et al., J. Exp. Med. 171:763-773, 1990). Further, it has also been shown that the immunity that developed in mice vaccinated with irradiated sporozoites is also dependent upon the present of CD8+ T cells. These T cells accumulate in inflammatory liver infiltrates subsequent to challenge. Passive transfer of circumsporozoite (CSP)-specific CTL clones as long as three hours after inoculation of sporozoites (i.e., after the parasites have left the bloodstream and infected liver cells) were capable of protecting animals against infection (Romero et al., Nature 341:323, 1989).

It is notable that CTL-restricted responses directed against a single antigen are insufficient to protect mice with different MHC alleles, and a combination of multiple antigens was required even to protect mice from the most common laboratory strains of Plasmodium. These data indicate that a combination of epitopes form several antigens is necessary to elicit a protective CTL response.

Indirect evidence that CTLs are important in protective immunity against Pf in humans has also accumulated. It has been reported that cytotoxic CD8⁺ T cells can be identified in humans immunized with PF sporozoites (Moreno, et al., Int. Immunol. 3:997, 1991). Further, humans immunized with irradiated sporozoites or naturally exposed to malaria can generate a CTL response to the pre-erythrocytic-stage antigens, CSP, sporozoite surface protein 2 (SSP2), liver-stage antigen-1 (LSA-1), and exported protein-1 (Exp-1) (see, e.g. Malik et al., Proc. Natl. Acad. Sci. USA 88, 3300-3304, 1991; Doolan et al., Int. Immunol. 3:511-516, 1991; Hill et al., Nature 360:434-439, 1992). Additionally, there is evidence that the polymorphism within the CSP may be the result of selection by CTLs of parasites that express variant forms (MCutchan and Water, Immunol. Lett. 25:23-26, 1990). This is based on the observation that the variation is nonsynonymous at the nucleotide level, thereby indicating selective pressure at the protein level. The polymorphism primarily maps to identified CTL and T helper epitopes (Doolan et al., Int. Immunol. 5:2746, 1993); and CTL responses to some of the parasite variants do not cross-react (Hill et al., supra). Finally, the MHC class I human leukocyte antigen (HLA)-Bw53 has been associated with resistance to severe malaria in The Gambia, and CTLs to a conserved epitope restricted by the HLA-Bw53 allele have been identified on P. falciparum LSA-1 (Hill et al., Nature 352:595-600, 1991; Hill et al., Nature 340:434-439, 1992). Since HLA-Bw53 is found in 15%-40% of the population of sub-Saharan Africa but in less than 1% of Caucasians and Asians, these data suggest evolutionary selection on the basis of protection against severe malaria.

Thus, antibody, and both HLA class I and class II restricted responses directed against multiple sporozoite antigens appear to be involved in generating protective immunity to malaria. Furthermore, several important antigenic epitopes against which humoral and cellular immunity is focused have already been exactly delineated.

In view of the heterogeneous immune response observed with PF infection, induction of a multi-specific cellular immune response directed simultaneously against multiple PF epitopes appears to be important for the development of an efficacious vaccine against PF. There is a need, however, to establish vaccine embodiments that elicit immune responses that correspond to responses seen in patients that clear PF infection.

Epitope-Based Vaccines. The use of epitope-based vaccines has several advantages over current vaccines. The epitopes for inclusion in such a vaccine are to be selected from conserved regions of viral or tumor-associated antigens, in order to reduce the likelihood of escape mutants. The advantage of an epitope-based approach over the use of whole antigens is that there is evidence that the immune response to whole antigens is directed largely toward variable regions of the antigen, allowing for immune escape due to mutations. Furthermore, immunosuppressive epitopes that may be present in whole antigens can be avoided with the use of epitope-based vaccines.

Additionally, with an epitope-based vaccine approach, there is an ability to combine selected epitopes (CTL and HTL) and additionally to modify the composition of the epitopes, achieving, for example, enhanced immunogenicity. Accordingly, the immune response can be modulated, as appropriate, for the target disease. Similar engineering of the response is not possible with traditional approaches.

Another major benefit of epitope-based immune-stimulating vaccines is their safety. The possible pathological side effects caused by infectious agents or whole protein antigens, which might have their own intrinsic biological activity, is eliminated.

An epitope-based vaccine also provides the ability to direct and focus an immune response to multiple selected antigens from the same pathogen. Thus, patient-by-patient variability in the immune response to a particular pathogen may be alleviated by inclusion of epitopes from multiple antigens from that pathogen in a vaccine composition. A “pathogen” may be an infectious agent or a tumor associated molecule.

One of the most formidable obstacles to the development of broadly efficacious epitope-based immunotherapeutics has been the extreme polymorphism of HLA molecules. In the past, effective non-genetically biased coverage of a population has been a task of considerable complexity; such coverage has required that epitopes be used specific for HLA molecules corresponding to each individual HLA allele. Therefore, impractically large numbers of epitopes would been required in order to cover ethnically diverse populations. Recently, methods have been developed that allow the identification of epitopes that bind multiple HLA molecules. Therefore, epitope-based vaccines can be designed that contain epitopes which, either individually or in combination, bind a greater number of HLA molecules. The resulting epitope-based vaccines have a greater breadth of population coverage across one or more continents and even worldwide.

Variation in Epitopes of Infectious Agents. A challenge in the development of effective vaccines against infectious agents such as hepatitis B virus (HBV) (47, 60) hepatitis C virus (HCV) (61-63), human papilloma virus (HPV) (64, 65) Plasmodium falciparum (66), and human immunodeficiency virus (HIV-1) is the protein sequence variation associated with different isolates. This variation is the result of gene sequence mutations. When such mutations occur in regions encoding epitopes recognized by cytotoxic T-lymphocytes (CTL), they provide a mechanism for escape of the agent from immune system control.

HIV-1 represents an infectious agent with an especially high frequency of sequence variation. The sequence variation associated with HIV-1 proteins from related isolates, members of the same clades or types, as well as unrelated isolates, is well documented (1). Viral escape from CTL induced as the result of natural infection or vaccines was documented in nonhuman primate models where the mechanism behind this escape was mutation of the primary anchor residues in dominant CTL epitopes (5-9). Viral escape from HIV-specific CTL has also been strongly implied by data obtained from HIV-1 infected individuals whose disease status change, including the transition from acute to chronic infection (10, 11), loss of stable control of viral replication and subsequent progression to AIDS (4, 12) or mother-to-child transmission (13). Thus, HIV-1 genetic and protein sequence variation represent a significant challenge to immune system-based control of viral replication, both within infected individuals and within populations.

While the public health need for a vaccine against HIV-1 is well recognized and accepted, the genetic variation of HIV-1 isolates represents a highly significant obstacle (1, 14-16). Several strategies have been proposed, some of which include:
(1) Designing vaccines on HIV-1 types prevalent within small, well defined populations or geographical regions, such as individual countries or regions, and producing multiple different vaccines for exclusive use within these countries or regions (16).
(2) Use of HIV-1 ancestral or consensus sequences based on HIV types present in larger target populations, such as groups of neighboring countries or continents (15, 17-19).
(3) Incorporation of viral gene products obtained from multiple different virus isolates, representing diversely different types or clades, into a single ‘multi-valent’ vaccine.

Related vaccine design concepts that incorporate many of the advantages associated with the approaches described above are the use of highly conserved regions or epitopes derived from these regions as the basis of the vaccine. The logic behind this approach is that conserved regions of the viral genome are those that have been maintained through the evolution of HIV-1 because changes impact gene product function and general viral fitness. This theory is consistent with analyses of HIV-1 protein sequence data which demonstrated that CTL epitopes are concentrated in conserved regions and that regions devoid of CTL epitopes are the most variable (20). Additional support comes from published reports describing CTL responses, induced as the result of natural infection or vaccination, that recognize viral proteins or epitopes common to viral isolates from diverse types or clades (21-26). Broad function CTL responses are also known to be correlated with slower progression to AIDS, at least for certain carefully studied populations (27, 28). Despite these reports and the clustering of CTL epitopes in conserved regions of HIV-1 gene products, amino acid sequence variation of analogous regions and epitopes from different viral isolates, both within the same type or lade and from different types, remains significant. There are currently no rules guiding the selection of conserved regions of CTL epitopes for use in vaccines other than the use of amino acid sequence identity (29).

A clear understanding of how CTL recognize pathogen infected cells has emerged over the past decade. It is now well established that small fragments of pathogen-derived proteins are generated, defined as peptide epitopes generally 8-11 amino acids in length, which bind to HLA-A, -B, or -C (human Class I Major Histocompatability Molecules) molecules expressed on the cell surface. Sequencing of naturally processed peptides bound to HLA molecules provided a means to identify the amino acid residues required for allele-specific epitope-peptide binding (30-32). Data obtained from X-ray crystallographic analysis of HLA-epitope peptide complexes, allowed for the identification and structural characterization of ‘binding pockets’ within the peptide binding cleft of HLA molecules. More refined epitope anchor motif definitions were then developed using data obtained from in vitro peptide-MHC binding assays. It is now well known that the main anchor residues typically occur at position 2 and the carboxyl terminus of peptides 8-11 amino acids in length, thus positions 8, 9, 10 or 11 (33-40). The definition of epitope peptide binding anchor motifs is the key to most, if not all, epitope prediction methods.

Initial CTL epitope identification methods were developed using common HLA alleles, such as HLA-A2.1. Motifs defined using different HLA molecules were found to be similar and this lead to the definition of HLA supertype families (41). The biological effect of this supertype relationship was first demonstrated for HIV-1 epitopes in a study where the HLA-A3 and -A11 epitope peptide binding patterns repertoires were demonstrated to be overlapping, not only with each other but also with HLA-A31, -A33 and -A*6801 (42). This binding specificity was defined as the HLA-A3 supertype. A significant overlap in peptide binding patterns was also demonstrated amongst several serologically distant HLA-B alleles (43, 44), and multiple HLA-A2 alleles (45, 46), resulting in the definition of the HLA-B7 and HLA-A2 supertype families. Recognition of epitopes by CTL in a supertype manner has since been demonstrated to occur naturally in infectious diseases and cancer (47-53).

While only two positions within CTL epitopes are typically characterized as the primary binding anchor positions, the amino acids that can serve as the anchor residues are more variable. The preferred and tolerated amino acids that can serve as anchor residues for the HLA-A2, -A3 and -B7 supertype families of epitopes are listed in Table 1. It is possible for analogous HIV-1 epitope peptides derived from different isolates, which differ with respect to the amino acids used as anchor residues, to bind to HLA molecules similarly. This type of variation can be as conserved since it is likely that CTL produced against one epitope would recognize the related epitope. Thus, variation limited to changes in anchor residues that result in sufficient epitope peptide binding to HLA molecules does not result in immune escape from CTL. Epitopes that contain this type of variation can be identified using the appropriately designed motif search algorithms.

The TCR of CTL has been reported to be somewhat flexible or promiscuous with respect to recognition of epitope peptides bound to HLA molecules. For HIV-1, this flexibility was demonstrated as CTL recognition of related, but slightly variable, epitopes by single clones of CTL produced following natural infection (54, 55). Similar flexibility of CTL epitope recognition was demonstrated using rhesus macaques and natural infection with SIV or immunization (56, 57). This observation is not unique to HIV-1 and SIV but rather the TCR appears to have evolved to allow promiscuous recognition of peptide epitope bound to MHC molecules (58).

Selective replacement of certain amino acids in CTL epitope peptides, amino acids thought to represent TCR contact points, is not only tolerated but can increase the recognition of the epitopes by CTL clones (59). The types of amino acid substitutions that can be incorporated, typically amino acids that are similar in chemical properties are best tolerated, and their positions, independent of primary anchor positions, within a selected number of CTL epitopes from tumor associated antigens were also defined.

For HIV-1 and other infectious agents, reproducible methods for predicting the CTL recognition of related variant epitopes that occur amongst isolates have not been developed. Nor have methods for identifying CTL epitopes that are most likely to induce broadly functional responses when used in vaccine. Thus, there exists a need to develop such methods to overcome the challenge associated with protein sequence variation in HIV and other infectious agents.
1. Åsjö, B., et al. AIDS 11:A17-A36. 1997.
2. Phillips, R. E., et al. Nature 354:453-459. 1991.
3. Phillips, R. E., et al. Chem. Immunol. 56:150-164. 1993.
4. McMichael, A. J., et al. Annu. Rev. Immunol. 15:271-296. 1997.
5. Allen, T. M., et al. Nature 407:386-390. 2000.
6. Barouch, D. H., et al. Nature 415:335-339. 2002.
7. Evans, D. T., et al. Nat. Med. 5:1270-1276. 1999.
8. Vogel, T. U., et al. J. Virol. 76:11623-11636. 2002.
9. Nacsa, J., et al. Virology 305:210-218. 2003.
10. Borrow, P., et al. Nat. Med. 3:205-211. 1997.
11. Price, D. A., et al. Proc. Natl. Acad. Sci. U.S.A 94:1890-1895. 1997.
12. Goulder, P. J., et al. Nat. Med. 3:212-217. 1997.
13. Goulder, P. J., et al. Immunol. Lett. 79:109-116. 2001.
14. Walker, B. D. et al. Nat. Immunol. 2:473-475. 2001.
15. Korber, B., et al. Br. Med. Bull. 58:19-42. 2001.
16. Gaschen, B., et al. Science 296:2354-2360. 2002.
17. Novitsky, V., et al. J. Virol. 76:5435-5451. 2002.
18. Davis, N. L., et al. IUBMB. Life 53:209-211. 2002.
19. Ellenberger, D. L., et al. Virology 302:155-163. 2002.
20. Yusim, K., et al. J. Virol. 76:8757-8768. 2002.
21. Ferrari, G., et al. Proc. Natl. Acad. Sci. U.S.A. 94:1396-1401. 1997.
22. Fukada, K., et al. AIDS 16:701-711. 2002.
23. Ferrari, G., et al. AIDS Res. Hum. Retroviruses 16:1433-1443. 2000.
24. Buseyne, F., et al. Virology 250:316-324. 1998.
25. Lynch, J. A., et al. J. Infect. Dis. 178:1040-1046. 1998.
26. Wilson, S. E., et al. AIDS Res. Hum. Retroviruses 14:925-937. 1998.
27. Rowland-Jones, S. L., et al. Immunol. Lett. 66:9-14. 1999.
28. Gillespie, G. M., et al. AIDS 16:961-972. 2002.
29. Altfeld, M. A., et al. J. Virol. 75:1301-1311. 2001.
30. Sette, A. et al. Curr. Opin. Immunol. 4:79-86. 1992.
31. Sinigaglia, F. et al. Curr. Opin. Immunol. 6:52-56. 1994.
32. Engelhard, V. H. Curr. Opin. Immunol. 6:13-23. 1994.
33. Brown, K., et al. Nature 364:33-39. 1994.
34. Guo, H. C., et al. Proc. Natl. Acad. Sci. U.S.A. 90:8053-8057. 1993.
35. Guo, H. C., et al. Nature 360:364-366. 1992.
36. Silver, M. L., et al. Nature 360:367-369. 1992.
37. Matsumura, M., et al. Science 257:927-934. 1992.
38. Madden, D. R., et al. Cell 70:1035-1048. 1995.
39. Fremont, D. H., et al. Science 257:919-927. 1992.
40. Sapp, M., et al. J. Mol. Biol. 219:277-319. 1991.
41. Kubo, R. T., et al. J. Immunol. 152:3913-24. 1994.
42. Sidney, J., et al. Hum. Immunol. 45:79-93. 1996.
43. Sidney, J., et al. J. Immunol. 154:247-259. 1995.
44. Sidney, J., et al. J. Immunol 157:3480-3490. 1996.
45. Del Guercio, M.-F., et al. J. Immunol. 154:685-693. 1995.
46. Fruci, D., et al. Hum. Immunol. 38:187-192. 1993.
47. Bertoni, R., et al. J. Clin. Invest. 100:503-513. 1997.
48. Threlkeld, S. C., et al. J. Immunol. 159:1648-1657.
49. Khanna, R., et al. J. Virol. 71:7429-7435. 1997.
50. Bertoletti, A., et al. Hepatology 26:1027-1034. 1997.
51. Fleischhauer, K., et al. J. Immunol. 157:787-297. 1996.
52. Kawashima, I., et al. Hum. Immunol. 59:1-14. 1998.
53. Wang, R. F., et al. J. Immunol. 160:890-897. 1998.
54. Tomiyama, H., et al. J. Immunol. 30:2521-2530. 2000.
55. Buseyne, F., et al. Int. Immunol. 13:941-950. 2001.
56. Shen, L., et al. J. Immunol. 153:12-15-1994. 2000.
57. Charini, W. A., et al. J. Immunol. 167:4996-5003. 2001.
58. Mason, D. Immunol. Today. 19:395-404. 1998.
59. Tangri, S., et al. J. Exp. Med. 194:833-846. 2001.
60. Rehermann, B., et al. J. Exp. Med. 181:1047-1058. 1995.
61. Chang, K. M., et al. J. Immunol. 162:1156-1164. 1999.
62. Chang, K. M., et al. J. Clin. Invest. 100:2376-2385. 1997.
63. Propato, A., et al. Hum. Immunol. 62:561-576. 2001.
64. Kast, W. M., et al. J. Immunol. 152:3904-3912. 1994.
65. Ressing, M. E., et al. J. Immunol. 154:5934-5943. 1995.
66. Doolan, D. L., et al. Immunity. 7:97-112. 1997.

### SUMMARY OF THE INVENTION

The present invention is directed to methods for selecting a variant of a peptide epitope which induces a CTL response against another variant(s) of the peptide epitope, by determining whether the variant comprises only conserved residues, as defined herein, at non-anchor positions in comparison to the other variant(s).

In some embodiments, antigen sequences from a population of an infectious agent, said antigens comprising variants of a peptide epitope, are optionally aligned (manually or by computer) along their length, preferably their full length. Variant(s) of a peptide epitope (preferably naturally occurring variants), each 8-11 amino acids in length and comprising the same MHC class I supermotif or motif, are identified manually or with the aid of a computer. In some embodiments, a variant is optionally chosen which comprises preferred anchor residues of said motif and/or which occurs with high frequency within the population of variants. In other embodiments, a variant is randomly chosen. The randomly or otherwise chosen variant is compared to from one to all the remaining variant(s) to determine whether it comprises only conserved residues in the non-anchor positions relative to from one to all the remaining variant(s).

The present invention is also directed to variants identified by the methods above; peptides comprising such variants; nucleic acids encoding such variants and peptides; cells comprising such variants, and/or peptides, and/or nucleic acids; compositions comprising such variants, and/or peptides, and/or nucleic acids, and/or cells; as well as therapeutic and diagnostic methods for using such variants, peptides, nucleic acids, cells, and compositions.

### BRIEF DESCRIPTION OF THE DRAWINGS/FIGURES

FIGS. 1A-1E. Recognition of variant peptides by CTL generated against a single epitope. Variant peptides were identified from 167 HIV strains for 5 HIV epitopes, 3 HLA-A2 restricted (Env 134, A, Gag 386, B, and Vpr 62, C) and 2 HLA-A11 restricted (Pol 98, D, and Env 47, E). These are listed according to their relationship to a previously determined parent (P) into single anchor substitutions (A), single non-anchor substitutions (NA) or multiple substitutions (M). Binding of each variant peptide is also shown. The number of viral sequences containing each variant peptide is shown in the column labeled # Isolates, and is reported for the total sequences, Clade B sequences (B), and Clade C sequences (C). Finally, the ability of CTL primed against the parent peptide to recognize the variant peptides is shown in the bar graphs.

FIGS. 2A-2C. Characterization of the peptide-specific T cell lines. A. FACS analysis of the TCRs expressed by peptide-stimulated cells after 0, 1, and 5 peptide stimulations, using a panel of commercially available mAb for mouse TCR 2-14. B-C. Peptide affinity. Parent and variant peptides were titrated against CTL that had been stimulated 5 times with the parent peptide.

FIGS. 2A-2B. Recognition of a panel of variant peptides by PBL from an HIV-infected individual.

FIG. 4. Prediction of immunological conservation. Gag 271 variants and their binding are shown, along with the number of isolates that express each variant. Immunological recognition was predicted for each variant based on two different choices in the immunizing peptide. On the right, the immunogenicity for each variant is shown.

### DETAILED DESCRIPTION OF THE INVENTION

Definitions

The invention can be better understood with reference to the following definitions:

An “antigen” refers to a polypeptide encoded by the genome of an infectious agent, or other another source, but preferably an infectious agent in the present invention. Examples of HIV antigens include Env, Gag, Nef, Pol, Tat, Rev, Vif, Vpr, Vpu, p17, p24, p2, p7, p1, p6, Protease, RT, Integrase, and gp160 (preferably Env, Gag, Nef, Pol, Tat, Rev, Vif, Vpr, Vpu). Examples of HIV antigens include Core, Env, and Pol. Examples of HCV antigens include Core, E1, E2, Ns1, Ns2, Ns3, Ns4, and Ns5. Examples of HPV antigens include E1, E2, E3, E4, E5, E6, E7, L1, and L2. Examples of Plasmodium falciparum antigens include CSP, SSP2, Exp1, and LSA1.

Throughout this disclosure, “binding data” results are often expressed in terms of “IC₅₀'s.” IC₅₀ is the concentration of peptide in a binding assay at which 50% inhibition of binding of a reference peptide is observed. Given the conditions in which the assays are run (i.e., limiting HLA proteins and labeled peptide concentrations), these values approximate K_{D} values. Assays for determining binding are described in detail, e.g., in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). It should be noted that IC₅₀ values can change, often dramatically, if the assay conditions are varied, and depending on the particular reagents used (e.g., HLA preparation, etc.). For example, excessive concentrations of HLA molecules will increase the apparent measured IC₅₀ of a given ligand.

Alternatively, binding is expressed relative to a reference peptide. Although as a particular assay becomes more, or less, sensitive, the IC₅₀'s of the peptides tested may change somewhat, the binding relative to the reference peptide will not significantly change. For example, in an assay run under conditions such that the IC₅₀ of the reference peptide increases 10-fold, the IC₅₀ values of the test peptides will also shift approximately 10-fold. Therefore, to avoid ambiguities, the assessment of whether a peptide is a good (i.e. high), intermediate, weak, or negative binder is generally based on its IC₅₀, relative to the IC₅₀ of a standard peptide. The Tables included in this application present binding data in a preferred biologically relevant form of IC₅₀ nM.

Binding may also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g. Hill et al., J. Immunol. 152, 2890 (1994); Marshall et al., J. Immunol 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896(1992)).

As used herein, “high affinity” with respect to HLA class I molecules is defined as binding with an IC₅₀ or K_{D} value, of 50 nM or less, “intermediate affinity” is binding with an IC₅₀ or K_{D} value of between 50 and about 500 nM, weak affinity is binding with an IC₅₀ or K_{D} value of between about 500 and about 5000 nM. “High affinity” with repect to binding to HLA class II molecules is defined as binding with an IC₅₀ or K_{D} value of 100 nM or less; “intermediate affinity” is binding with an IC₅₀ or K_{D} value of between about 100 and about 1000 nM.

A “computer” or “computer system” generally includes: a processor and related computer programs; at least one information storage/retrieval apparatus such as a hard drive, a disk drive or a tape drive; at least one input apparatus such as a keyboard, a mouse, a touch screen, or a microphone; and display structure, such as a screen or a printer. Additionally, the computer may include a communication channel in communication with a network. Such a computer may include more or less than what is listed above.

“Cross-reactive binding” indicates that a peptide is bound by more than one HLA molecule; a synonym is degenerate binding.

A “cryptic epitope” elicits a response by immunization with an isolated peptide, but the response is not cross-reactive in vitro when intact whole protein, which comprises the epitope, is used as an antigen.

The term “derived” when used to discuss an epitope is a synonym for “prepared.” A derived epitope can be isolated from a natural source, or it can be synthesized in accordance with standard protocols in the art. Synthetic epitopes can comprise artificial amino acids “amino acid mimetics,” such as D isomers of natural occurring L amino acids or non-natural amino acids such as cyclohexylalanine. A derived/prepared epitope can be an analog of a native epitope.

A “diluent” includes sterile liquids, such as water and oils, including those of petroleum, animal, vegetable or synthetic origin, such as peanut oil, soybean oil, mineral oil, sesame oil and the like. Water is a preferred diluent for pharmaceutical compositions. Saline solutions and aqueous dextrose and glycerol solutions can also be employed as diluents, particularly for injectable solutions.

A “dominant epitope” is an epitope that induces an immune response upon immunization with a whole native antigen (see, e.g., Sercarz, et al., Annu. Rev. Immunol 11:729-766, 1993). Such a response is cross-reactive in vitro with an isolated peptide epitope.

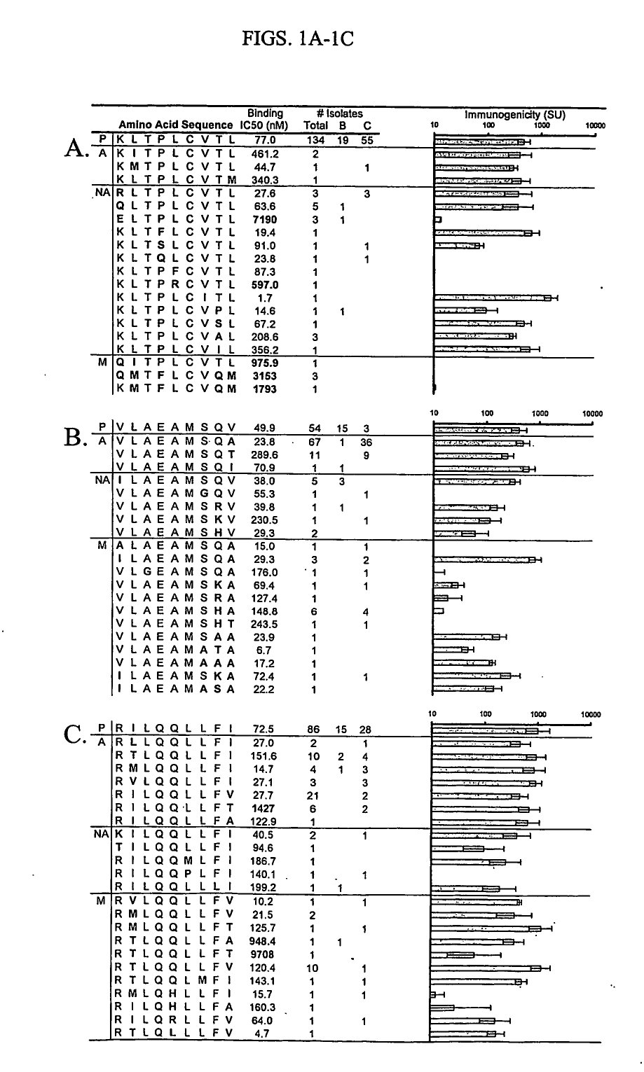

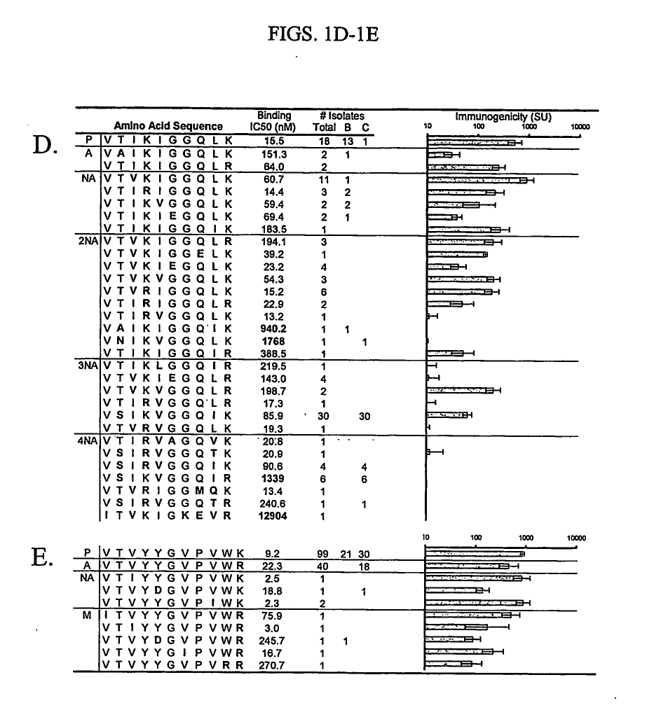

An “epitope” is the collective features of a molecule, such as primary, secondary and tertiary peptide structure, and charge, that together form a site recognized by an immunoglobulin, T cell receptor or HLA molecule. Alternatively, an epitope can be defined as a set of amino acid residues which is involved in recognition by a particular immunoglobulin, or in the context of T cells, those residues necessary for recognition by T cell receptor proteins and/or Major Histocompatibility Complex (MHC) receptors. Epitopes are present in nature, and can be isolated, purified or otherwise prepared/derived by humans. For example, epitopes can be prepared by isolation from a natural source, or they can be synthesized in accordance with standard protocols in the art. Synthetic epitopes can comprise artificial amino acids, “amino acid mimetics,” such as D isomers of naturally-occurring L amino acids or non-naturally-occuring amino acids such as cyclohexylalanine. Throughout this disclosure, epitopes may be referred to in some cases as peptides. The variants of the invention are set forth in Tables 6-9 and FIGS. 1A-4.

It is to be appreciated that proteins or peptides that comprise a variant of the invention as well as additional amino acid(s) are still within the bounds of the invention. In certain embodiments, the peptide comprises a fragment of an antigen. A “fragment of an antigen” or “antigenic fragment” or simply “fragment” is a portion of an antigen which has 100% identity with a wild type antigen or naturally-ocurring variant thereof. The fragment may or may not comprise an epitope of the invention. The fragment may be less than or equal to 600 amino acids, less than or equal to 500 amino acids, less than or equal to 400 amino acids, less than or equal to 250 amino acids, less than or equal to 100 amino acids, less than or equal to 85 amino acids, less than or equal to 75 amino acids, less than or equal to 65 amino acids, or less than or equal to 50 amino acids in length. In certain embodiments, a fragment is e.g., less than 101 or less than 51 amino acids in length, in any increment down to 5 amino acids in length. For example, the fragment may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length.

In certain embodiments, there is a limitation on the length of a peptide of the invention. The embodiment that is length-limited occurs when the protein/peptide comprising an epitope of the invention comprises a region (i.e., a contiguous series of amino acids) having 100% identity with a native sequence. In order to avoid the definition of epitope from reading, e.g., on whole natural molecules, there is a limitation on the length of any region that has 100% identity with a native peptide sequence. Thus, for a peptide comprising an epitope of the invention and a region with 100% identity with a native peptide sequence, the region with 100% identity to a native sequence generally has a length of: less than or equal to 600 amino acids, often less than or equal to 500 amino acids, often less than or equal to 400 amino acids, often less than or equal to 250 amino acids, often less than or equal to 100 amino acids, often less than or equal to 85 amino acids, often less than or equal to 75 amino acids, often less than or equal to 65 amino acids, and often less than or equal to 50 amino acids. In certain embodiments, an “epitope” of the invention is comprised by a peptide having a region with less than 51 amino acids that has 100% identity to a native peptide sequence, in any increment down to 5 amino acids.

Accordingly, peptide or protein sequences longer than 600 amino acids are within the scope of the invention, so long as they do not comprise any contiguous sequence of more than 600 amino acids that have 100% identity with a native peptide sequence. For any peptide that has five contiguous residues or less that correspond to a native sequence, there is no limitation on the maximal length of that peptide in order to fall within the scope of the invention. It is presently preferred that a peptide of the invention (e.g., a peptide comprising an epitope of the invention) be less than 600 residues long in any increment down to eight amino acid residues.

A peptide epitope occurring with “high frequency” is one that occurs in at least 30%, at least 40%, at least 50%, at least 60%, at least 70%, at least 80%, or at least 90% of the infectious agents in a population. A “high frequency” peptide epitope is one of the more common in a population, preferably the first most common, second most common, third most common, or fourth most common in a population of variant peptide epitopes.

“Human Leukocyte Antigen” or “HLA” is a human class I or class II Major Histocompatibility Complex (MHC) protein (see, e.g., Stites, et al., IMMUNOLOGY, 8^{TH} ED., Lange Publishing, Los Altos, Calif. (1994).

An “HLA supertype or HLA family”, as used herein, describes sets of HLA molecules grouped on the basis of shared peptide-binding specificities. HLA class I molecules that share somewhat similar binding affinity for peptides bearing certain amino acid motifs are grouped into such HLA supertypes. The terms HLA superfamily, HLA supertype family, HLA family, and HLA xx-like molecules (where “xx” denotes a particular HLA type), are synonyms. See Tables 1-4.

As used herein, “high affinity” with respect to HLA class I molecules is defined as binding with an IC₅₀, or K_{D} value, of 50 nM or less; “intermediate affinity” is binding with an IC₅₀ or K_{D} value of between about 50 and about 500 nM; “weak affinity” is binding with an IC₅₀ or K_{D} value between about 500 and about 5000 nM. “High affinity” with respect to binding to HLA class II molecules is defined as binding with an IC₅₀ or K_{D} value of 100 nM or less; “intermediate affinity” is binding with an IC₅₀ or K_{D} value of between about 100 and about 1000 nM. See “binding data.”

An “IC₅₀” is the concentration of peptide in a binding assay at which 50% inhibition of binding of a reference peptide is observed. Given the conditions in which the assays are run (i.e., limiting HLA proteins and labeled peptide concentrations), these values approximate K_{D} values. See “binding data.”

The terms “identical” or percent “identity,” in the context of two or more peptide sequences or antigen fragments, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

An “immunogenic” peptide or an “immunogenic” epitope or “peptide epitope” is a peptide that comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T lymphocyte (CTL) response, or a helper T lymphocyte (HTL) response, to the peptide.

An “infectious agent” refers to a disease-causing microorganism, including viruses, bacteria, fungi, and protozoa against which a cellular immune response, preferably a CTL response, plays a role in acquired immunity. Examples of infectious agents include viruses such as human immunodeficiency virus (HIV), hepatitis B virus (HBV), hepatitis C virus (HCV), human papillomma virus (HPV), Influenza virus, Dengue virus, Epstein-Barr virus, bacteria such as Mycobacterium tuberculosis and Chlamydia, fungi such as Candida albicans, Cryptococcus neoformans, Coccidoides spp., Histoplasma spp, and Aspergillus fumigatis, protozoa such as Plasmodium spp., including P. falciparum, Trypanosoma spp., Schistosoma spp., Leishmania spp and the like. Preferred infectious agents include HIV, HBV, HCV, HPV, Epstein-Barr virus, Plasmodium falciparum, Influenza virus and Dengue virus.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment. An “isolated” epitope refers to an epitope that does not include the whole sequence of the antigen or polypeptide from which the epitope was derived. Typically the “isolated” epitope does not have attached thereto additional amino acids that result in a sequence that has 100% identity with a native sequence. The native sequence can be a sequence such as a tumor-associated antigen from which the epitope is derived. Thus, the term “isolated” means that the material is removed from its original environment (e.g., the natural environment if it is naturally occurring). For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules of the present invention, and further include such molecules produced synthetically.

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, FUNDAMENTAL IMMUNOLOGY, 3^{RD} ED., Raven Press, New York (1993).

The term “motif” refers to a pattern of residues in an amino acid sequence of defined length, preferably a peptide of less than about 15 amino acids in length, or less than about 13 amino acids in length, usually from about 8 to about 13 amino acids (e.g., 8, 9, 10, 11, 12, or 13) for a class I HLA motif and from about 6 to about 25 amino acids (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs often differ in their pattern of the primary and secondary anchor residues. See Tables 1-3.

A “native” or a “wild type” sequence refers to a sequence found in nature.

A “negative binding residue” or “deleterious residue” is an amino acid which, if present at certain positions (typically not primary anchor positions) in a peptide epitope, results in decreased binding affinity of the peptide for the peptide's corresponding HLA molecule.

The term “peptide” is used interchangeably with “oligopeptide” in the present specification to designate a series of residues, typically L-amino acids, connected one to the other, typically by peptide bonds between the α-amino and carboxyl groups of adjacent amino acids.

A “PanDR binding” peptide or “PADRE®” peptide (Epimmune, San Diego, Calif.) is a member of a family of molecules that binds more than one HLA class II DR molecule. The pattern that defines the PADRE® family of molecules can be referred to as an HLA Class II supermotif. A PADRE® molecule binds to HLA-DR molecules and stimulates in vitro and in vivo human helper T lymphocyte (HTL) responses. For a further definition of the PADRE® family, see copending application U.S. Ser. No. 09/709,774, filed Nov. 11, 2000; and Ser. No. 09/707,738, filed Nov. 6, 2000; PCT publication Nos WO 95/07707, and WO 97/26784; U.S. Pat. No. 5,736,142 issued Apr. 7, 1998; U.S. Pat. No. 5,679,640, issued Oct. 21, 1997; and U.S. Pat. No. 6,413,935, issued Jul. 2, 2002.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

A “primary anchor residue” is an amino acid at a specific position along a peptide sequence which is understood to provide a contact point between the immunogenic peptide and the HLA molecule. One, two or three, primary anchor residues within a peptide of defined length generally defines a “motif” for an immunogenic peptide. These residues are understood to fit in close contact with peptide binding grooves of an HLA molecule, with their side chains buried in specific pockets of the binding grooves themselves. In one embodiment of an HLA class I motif, the primary anchor residues are located at position 2 (from the amino terminal position) and at the carboxyl terminal position of a peptide epitope in accordance with the invention. The primary anchor positions for each motif and supermotif of HLA Class I are set forth in Tables 1-2. For example, analog peptides can be created by altering the presence or absence of particular residues in these anchor positions. Such analogs are used to modulate the binding affinity of an epitope comprising a particular motif or supermotif. A “preferred primary anchor residue” is an anchor residue of a motif or supermotif that is associated with optimal binding. Preferred primary anchor residues are indicated in bold-face in Tables 1-2. A “tolerated primary anchor residue” is an anchor residue of a motif or supermotif that is associated with binding to a lesser extent than a preferred residue. Tolerated primary anchor residues are indicated in italicized text in Tables 1-2.

“Promiscuous recognition” by a TCR is where a distinct peptide is recognized by the various T cell clones in the context of various HLA molecules. Promiscuous binding by an HLA molecule is synonymous with cross-reactive binding.

A “protective immune response” or “therapeutic immune response” refers to a CTL and/or an HTL response to an antigen derived from an antigen of an infectious agent, which in some way prevents or at least partially arrests disease symptoms, side effects or progression. The immune response may also include an antibody response which has been facilitated by the stimulation of helper T cells.

By “ranking” the variants in a population of peptide epitopes is meant ordering each variant by its frequency of occurrance relative to the other variants.

The term “residue” refers to an amino acid or amino acid mimetic incorporated into a peptide or protein by an amide bond or amide bond mimetic.

A “secondary anchor residue” is an amino acid at a position other than a primary anchor position in a peptide which may influence peptide binding. A secondary anchor residue occurs at a significantly higher frequency amongst HLA-bound peptides than would be expected by random distribution of amino acids at a given position. A secondary anchor residue can be identified as a residue which is present at a higher frequency among high or intermediate affinity binding peptides, or a residue otherwise associated with high or intermediate affinity binding. The secondary anchor residues are said to occur at “secondary anchor positions.” For example, analog peptides can be created by altering the presence or absence of particular residues in these secondary anchor positions. Such analogs are used to finely modulate the binding affinity of an epitope comprising a particular motif or supermotif. The terminology “fixed peptide” is generally used to refer to an analog peptide that has changes in primary anchore position; not secondary.

A “subdominant epitope” is an epitope which evokes little or no response upon immunization with a whole antigen or a fragment of the whole antigen comprising a subdominant epitope and a dominant epitope, which comprise the epitope, but for which a response can be obtained by immunization with an isolated peptide, and this response (unlike the case of cryptic epitopes) is detected when whole antigen or a fragment of the whole antigen comprising a subdominant epitope and a dominant epitope is used to recall the response in vitro or in vivo.

A “supermotif” is a peptide binding specificity shared by HLA molecules encoded by two or more HLA alleles. Preferably, a supermotif-bearing peptide is recognized with high or intermediate affinity (as defined herein) by two or more HLA antigens.

“Synthetic peptide” refers to a peptide that is abtained from a non-natural source, e.g. is man-made. Such peptides may be produced using such methods as chemical synthesis or recombinant DNA technology. “Synthetic peptides” include “fusion proteins.”

As used herein, a “vaccine” is a composition used for vaccination, e.g., for prophylaxis or therapy, that comprises one or more peptides of the invention. There are numerous embodiments of vaccines in accordance with the invention, such as by a cocktail of one or more peptides; one or more peptides of the invention comprised by a polyepitopic peptide; or nucleic acids that encode such peptides or polypeptides, e.g., a minigene that encodes a polyepitopic peptide. The “one or more peptides” can include any whole unit integer from 1-150, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120, 125, 130, 135, 140, 145, or 150 or more peptides of the invention. The peptides or polypeptides can optionally be modified, such as by lipidation, addition of targeting or other sequences. HLA class I-binding peptides of the invention can be linked to HLA class II-binding peptides, e.g., a PADRE® universal HTL-bindind peptide, to facilitate activation of both cytotoxic T lymphocytes and helper T lymphocytes. Vaccines can comprise peptide pulsed antigen presenting cells, e.g., dendritic cells.

A “variant of a peptide epitope” refers to a peptide that is identified from a different viral strain at the same position in an aligned sequence, and that varies by one or more amino acids from the parent peptide epitope. Examples of peptide epitope variants include those shown in Tables 6-9 and FIGS. 1A-4. A “variant of an antigen” refers to an antigen that comprises at least one variant of a peptide epitope. Examples of antigen variants include those listed by sequence and/or accession number in Tables 10-22. A “variant of an infectious agent” refers to an infectious agent whose genome encodes at least one variant of an antigen. Variants of infectious agents are related viral, bacterial, funagl, or protozoan strains or isolates that vary in sequence but cause the same disease symptoms. Examples of infectious agent variants include HIV Clade A, B, and C subtypes, HBV subtypes adr, ayr, adw, and ayw, HCV types 1, 2, 3, 4, 5, and 6, HPV strains 1-92 (preferably strains 16, 18, 31, 33, 45, 52, 56, and 58) (see Table 10, listing accession numbers for the complete genome sequences of 167 HIV variants; Table 22, showing an alignment of the complete polyprotein sequences of 50 HCV variants) (see also, Human Retroviruses and AIDS 2000: A Compilation and Analysis of Nucleic Acid and Amino Acid Sequences, Kuiken C L, et al., Eds. Theoretical Biology and Biophysics Group, Los Alamos National Laboratory, Los Alamos, N. Mex.).

The nomenclature used to describe peptides/proteins follows the conventional practice wherein the amino group is presented to the left (the N-terminus) and the carboxyl group to the right (the C-terminus) of each amino acid residue. When amino acid residue positions are referred to in a peptide epitope they are numbered in an amino to carboxyl direction with position one being the position closest to the amino terminal end of the epitope, or the peptide or protein of which it may be a part. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. However, when three letter symbols or full names are used without capitals, they may refer to L amino acids. Glycine has no asymmetric carbon atom and is simply referred to as “Gly” or “G”. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.) In addition to these symbols, “B”in the single letter abbreviations used herein designates α-amino butyric acid. In some embodiments, α-amino butyric acid may be replaced with cysteine.

Acronyms used herein are as follows:
APC: Antigen presenting cell
CD3: Pan T cell marker
CD4: Helper T lymphocyte marker
CD8: Cytotoxic T lymphocyte marker
CEA: Carcinoembryonic antigen (see, e.g., SEQ ID NO: 363)
CTL: Cytotoxic T lymphocyte
DC: Dendritic cells. DC functioned as potent antigen presenting cells by stimulating cytokine release from CTL lines that were specific for a model peptide derived from hepatitis B virus. In vivo experiments using DC pulsed ex vivo with an HBV peptide epitope have stimulated CTL immune responses in vivo following delivery to naïve mice.
DLT: Dose-limiting toxicity, an adverse event related to therapy.
DMSO: Dimethylsulfoxide
ELISA: Enzyme-linked immunosorbant assay
E:T: Effector:Target ratio
G-CSF: Granulocyte colony-stimulating factor
GM-CSF: Granulocyte-macrophage (monocyte)-colony stimulating factor
HBV: Hepatitis B virus
HER2/neu: A tumor associated antigen; c-erbB-2 is a synonym (see, e.g., SEQ ID NO: 364)
HLA: Human leukocyte antigen
HLA-DR: Human leukocyte antigen class II
HPLC: High Performance Liquid Chromatography
HTC: Helper T Cell
HTL: Helper T Lymphocyte. A synonym for HTC.
ID: Identity
IFNγ: Interferon gamma
IL-4: Interleukin-4
IV: Intravenous
LU_{30%}: Cytotoxic activity for 10⁶ effector cells required to achieve 30% lysis of a target cell population, at a 100:1 (E:T) ratio.
MAb: Monoclonal antibody
MAGE: Melanoma antigen (see, e.g., SEQ ID NO: 365 and 366 for MAGE2 and MAGE3)
MLR: Mixed lymphocyte reaction
MNC: Mononuclear cells
PB: Peripheral blood
PBMC: Peripheral blood mononuclear cell
ProGP™: Progenipoietin™ product (Searle, St. Louis, Mo.), a chimeric flt3/G-CSF receptor agonist.
SC: Subcutaneous
S.E.M.: Standard error of the mean
QD: Once a day dosing
TAA: Tumor Associated Antigen
TNF: Tumor necrosis factor
WBC: White blood cells

The following describes the peptides, nucleic acid molecules, compositions, and methods of the invention in more detail.

Methods of Identifying Candidate Peptide Epitopes

The present invention is directed to methods for selecting a variant of a peptide epitope which induces a CTL response against another variant(s) of the peptide epitope, by determining whether the variant comprises only conserved residues, as defined herein, at non-anchor positions in comparison to the other variant(s).

In some embodiments, antigen sequences from a population of an infectious agent, said antigens comprising variants of a peptide epitope, are optionally aligned (manually or by computer) along their length, preferably their full length. Variant(s) of a peptide epitope (preferably naturally occurring variants), each 8-11 amino acids in length and comprising the same MHC class I supermotif or motif, are identified manually or with the aid of a computer. In some embodiments, a variant is optionally chosen which comprises preferred anchor residues of said motif and/or which occurs with high frequency within the population of variants. In other embodiments, a variant is randomly chosen. The randomly or otherwise chosen variant is compared to from one to all the remaining variant(s) to determine whether it comprises only conserved residues in the non-anchor positions relative to from one to all the remaining variant(s).

The present invention is also directed to variants identified by the methods above; peptides comprising such variants; nucleic acids encoding such variants and peptides; cells comprising such variants, and/or peptides, and/or nucleic acids; compositions comprising such variants, and/or peptides, and/or nucleic acids, and/or cells; as well as therapeutic and diagnostic methods for using such variants, peptides, nucleic acids, cells, and compositions.

In some embodiments, the invention is directed to a method for identifying a candidate peptide epitope which induces a HLA class I CTL response against variants of said peptide epitope, comprising
a) identifying, from a particular antigen of an infectious agent, variants of a peptide epitope 8-11 amino acids in length, each variant comprising primary anchor residues of the same HLA class I binding motif; and
b) determining whether one of said variants comprises only conserved non-anchor residues in comparison to at least one remaining variant, thereby identifying a candidate peptide epitope.

In some embodiments, (b) comprises identifying a variant which comprises only conserved non-anchor residues in comparison to at least 25%, at least 50%, at least 75%, at least 80%, at least 85%, at least 90%, at least 95%, at least 97%, or at least 99% of the remaining variants.

In some embodiments, the invention is directed to a method for identifying a candidate peptide epitope which induces a HLA class I CTL response against variants of said peptide epitope, comprising
a) identifying, from a particular antigen of an infectious agent, variants of a peptide epitope 8-11 amino acids in length, each variant comprising primary anchor residues of the same HLA class I binding motif;
b) determining whether each of said variants comprises conserved, semi-conserved or non-conserved non-anchor residues in comparison to each of the remaining variants; and
c) identifying a variant which comprises only conserved non-anchor residues in comparison to at least one remaining variant.

In some embodiments, (c) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

In some embodiments, the invention is directed to a method for identifying a candidate peptide epitope which induces a HLA class I CTL response against variants of said peptide epitope, comprising
a) identifying, from a particular antigen of an infectious agent, a population of variants of a peptide epitope 8-11 amino acids in length, each peptide epitope comprising primary anchor residues of the same HLA class I binding motif;
b) choosing a variant selected from the group consisting of:
   i) a variant which comprises preferred primary anchor residues of said motif; and
   ii) a variant which occurs with high frequency within the population of variants; and
c) determining whether the variant of (b) comprises only conserved non-anchor residues in comparison to at least one remaining variant, thereby identifying a candidate peptide epitope.

In some embodiments, (c) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

In some embodiments, the invention is directed to method for identifying a candidate peptide epitope which induces a HLA class I CTL response against variants of said peptide epitope, comprising
a) identifying, from a particular antigen of an infectious agent, a population of variants of a peptide epitope 8-11 amino acids in length, each peptide epitope comprising primary anchor residues of the same HLA class I binding motif;
b) choosing a variant selected from the group consisting of:
   i) a variant which comprises preferred primary anchor residues of said motif; and
   ii) a variant which occurs with high frequency within the population of variants; and
c) determining whether the variant of (b) comprises conserved, semi-conserved or non-conserved non-anchor residues in comparison to each of the remaining variants; and
d) identifying a variant which comprises only conserved non-anchor residues in comparison to at least one remaining variant.

In some embodiments, (d) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

In some embodiments, (a) comprises aligning the sequences of said antigens.

In some embodiments, (b) comprises comprises choosing a variant which comprises preferred primary anchor residues of said motif.

In some embodiments, (b) comprises comprises choosing a variant which occurs with high frequency within said population.

In some embodiments, (b) comprises ranking said variants by frequency of occurrence within said population.

In some embodiments, (b) comprises choosing a variant which comprises preferred primary anchor residues of said motif and which occurs with high frequency within said population.

In some embodiments, (b) comprises ranking said variants by frequency of occurrence within said population.

In some embodiments, the identified variant comprises the fewest conserved anchor residues in comparison to each of the remaining variants.

In some embodiments, the remaining variants comprise 1, 2, 3, 4, 5, 6, 7, 8, 9, 10, 11, 12, 13, 14, 15, 16, 17, 18, 19, 20, 21, 22, 23, 24, 25, 26, 27, 27, 28, 30, 35, 40, 45, 50, 55, 60, 65, 70, 75, 80, 85, 90, 95, 100, 110, 120, 130, 140, 150, 160, 170, 180, 190, 200, 220, 240, 260, 280, or 300 variants.

In some embodiments, the infectious agent is selected from the group consisting of: HIV, HBV, HCV, HPV, Plasmodium falciparum, Influenza virus, and Dengue virus, Epstein-Barr virus, Mycobacterium tuberculosis, Chlamydia, Candida albicans, Cryptococcus neoformans, Coccidoides spp., Histoplasma spp, Aspergillus fumigatis, Plasmodium spp., Trypanosoma spp., Schistosoma spp., and Leishmania spp.

In some embodiments, the infectious agent is selected from the group consisting of: HIV, HBV, HCV, HPV, Plasmodium falciparum, Influenza virus, and Dengue virus.

In some embodiments, the infectious agent is HIV and the antigen is selected from the group consisting of: Gag, Env, Pol, Nef, Rev, Tat, Vif, Vpr, and Vpu.

In some embodiments, the infectious agent is HBV and the antigen is selected from the group consisting of: Pol, Env, Core, and NS1/Env2.

In some embodiments, the infectious agent is HCV and the antigen is selected from the group consisting of: Core, E1, E2, NS1, NS2, NS3, NS4, and NS5.

In some embodiments, the infectious agent is HPV and the antigen is selected from the group consisting of: E1, E2, E3, E4, E5, E6, E7, L1, and L2.

In some embodiments, the infectious agent is Plasmodium falciparum and the antigen is selected from the group consisting of: CSP, SSP2, EXP1, LSA1.

In some embodiments, the selected variant and the at least one remaining variant comprise different primary anchor residues of the same motif or supermotif.

In some embodiments, the motif or supermotif is selected from the group consisting of those in Tables 1-2.

In some embodiments, the conserved non-anchor residues are at any of positions 3-7 of said variant.

In some embodiments, the variant comprises only 1-3 conserved non-anchor residues compared to at least one remaining variant.

In some embodiments, the variant comprises only 1-2 conserved non-anchor residues compared to at least one remaining variant.

In some embodiments, the variant comprises only 1 conserved non-anchor residue compared to at least one remaining variant.

In some embodiments, the infectious agent is HPV, and further wherein, the HPV infectious agent is selected from the group consisting of HPV strains 16, 18, 31, 33, 45, 52, 56, and 58.

In some embodiments, the variants are a population of naturally occurring variants.

Optional Alignment. Optionally, antigen sequences, either full-length or partial, may be aligned mannually or by computer. Convenient computer programs for aligning multiple sequences include Omiga, Oxford software, version 1.1.3, using ClustalW alignment, using an open gap penalty of 10.0, extend gap penalty of 0.05, and delay divergent sequences of 40.0 (See, e.g., Table 21); and BLASTP 2.2.5 (Nov. 16, 2002) (Altschul, S. F., et al., Nucleic Acids Res. 25:3389-3402 (1997)) using a cutoff=3e-88 (to select human sequences) (see, e.g., Table 20). Alternatively, alignments may be obtained through publicly available sources such as published journal articles and published patent documents or as disclosed herein (see, e.g., Tables 10-22).

HLA Class I Motifs Indicative of CTL Inducing Peptide Epitopes. A large fraction of HLA class I and class II molecules can be classified into a relatively few supertypes, each respective supertype characterized by largely overlapping peptide binding repertoires, and consensus structures of the main peptide binding pockets. Thus, peptides of the present invention are preferably identified by the primary residues of any one of several HLA-specific amino acid motifs, or if the presence of the motif corresponds to the ability to bind several allele-specific HLA antigens, a supermotif (see, e.g., Tables 1-2). The preferred primary residues are indicated in bold, while the tolerated primary residues are indicated by italics.

The primary anchor residues of the HLA class I peptide epitope supermotifs and motifs are summarized in Tables 1-2. Preferred primary anchors are shown in bold, while tolerated primary anchors are shown in italics. Primary and secondary anchor positions for HLA Class I are summarized in Table 3. Allele-specific HLA molecules that fall within the various HLA class I supertypes are listed in Table 4. In some cases, patterns of amino acid residues are present in both a motif and a supermotif. The relationship of a particular motif and any related supermotif is indicated in the description of the individual motifs.

Thus, the peptide motifs and supermotifs described below, and summarized in Tables 1-2, provide guidance for the identification and use of peptide epitopes comprising primary anchor residues of motifs or supermotifs in accordance with the invention.

Allele-specific HLA molecules that comprise HLA class I supertype families are listed in Table 4.

HLA-A1 supermotif. The HLA-A1 supermotif is characterized by the presence in peptide ligands of a small (T or S) or hydrophobic (L, I, V, or M) primary anchor residue in position 2, and an aromatic (Y, F, or W) primary anchor residue at the C-terminal position of the epitope. The corresponding family of HLA molecules that bind to the A1 supermotif (i.e., the HLA-A1 supertype) is comprised of at least A*0101, A*2601, A*2602, A*2501, and A*3201 (see, e.g., DiBrino, M. et al., J. Immunol. 151:5930, 1993; DiBrino, M. et al., J. Immunol. 152:620, 1994; Kondo, A. et al., Immunogenetics 45:249, 1997). Other allele-specific HLA molecules predicted to be members of the A1 superfamily are shown in Table 4. Peptides binding to each of the individual HLA proteins can be modulated by substitutions at primary and/or secondary anchor positions, preferably choosing respective residues specified for the supermotif.

HLA-A2 supermotif. Primary anchor specificities for allele-specific HLA-A2.1 molecules (see, e.g., Falk et al., Nature 351:290-296, 1991; Hunt et al., Science 255:1261-1263, 1992; Parker et al., J. Immunol. 149:3580-3587, 1992; Ruppert et al., Cell 74:929-937, 1993) and cross-reactive binding among HLA-A2 and -A28 molecules have been described. (See, e.g., Fruci et al., Human Immunol. 38:187-192, 1993; Tanigaki et al., Human Immunol 39:155-162, 1994; Del Guercio et al., J. Immunol. 154:685-693, 1995; Kast et al., J. Immunol. 152:3904-3912, 1994 for reviews of relevant data.) These primary anchor residues define the HLA-A2 supermotif; which presence in peptide ligands corresponds to the ability to bind several different HLA-A2 and -A28 molecules. The HLA-A2 supermotif comprises peptide ligands with L, I, V, M, A, T, or Q as a primary anchor residue at position 2 and L, I, V, M, A, or T as a primary anchor residue at the C-terminal position of the epitope.

The corresponding family of HLA molecules (i.e., the HLA-A2 supertype that binds these peptides) is comprised of at least: A*0201, A*0202, A*0203, A*0204, A*0205, A*0206, A*0207, A*0209, A*0214, A*6802, and A*6901. Other allele-specific HLA molecules predicted to be members of the A2 superfamily are shown in Table 4. As explained in detail below, binding to each of the individual allele-specific HLA molecules can be modulated by substitutions at the primary anchor and/or secondary anchor positions, preferably choosing respective residues specified for the supermotif.

The motifs comprising the primary anchor residues V, A, T, or Q at position 2 and L, I, V, A, or T at the C-terminal position are those most particularly relevant to the invention claimed herein.

HLA-A3 supermotif. The HLA-A3 supermotif is characterized by the presence in peptide ligands of A, L, I, V, M, S, or, T as a primary anchor at position 2, and a positively charged residue, R or K, at the C-terminal position of the epitope, e.g. in position 9 of 9-mers (see, e.g., Sidney et al., Hum. Immunol. 45:79, 1996). Exemplary members of the corresponding family of HLA molecules (the HLA-A3 supertype) that bind the A3 supermotif include at least A*0301, A*1101, A*3101, A*3301, and A*6801. Other allele-specific HLA molecules predicted to be members of the A3 supertype are shown in Table 4. As explained in detail below, peptide binding to each of the individual allele-specific HLA proteins can be modulated by substitutions of amino acids at the primary and/or secondary anchor positions of the peptide, preferably choosing respective residues specified for the supermotif.

HLA-A24 supermotif. The HLA-A24 supermotif is characterized by the presence in peptide ligands of an aromatic (F, W, or Y) or hydrophobic aliphatic (L, I, V, M, or T) residue as a primary anchor in position 2, and Y, F, W, L, I, or M as primary anchor at the C-terminal position of the epitope (see, e.g., Sette and Sidney, Immunogenetics, in press, 1999). The corresponding family of HLA molecules that bind to the A24 supermotif (i.e., the A24 supertype) includes at least A*2402, A*3001, and A*2301. Other allele-specific HLA molecules predicted to be members of the A24 supertype are shown in Table 4. Peptide binding to each of the allele-specific HLA molecules can be modulated by substitutions at primary and/or secondary anchor positions, preferably choosing respective residues specified for the supermotif.

HLA-B7 supermotif. The HLA-B7 supermotif is characterized by peptides bearing proline in position 2 as a primary anchor, and a hydrophobic or aliphatic amino acid (L, I, V, M, A, F, W, or Y) as the primary anchor at the C-terminal position of the epitope. The corresponding family of HLA molecules that bind the B7 supermotif (i.e., the HLA-B7 supertype) is comprised of at least twenty six HLA-B proteins including: B*0702, B*0703, B*0704, B*0705, B*1508, B*3501, B*3502, B*3503, B*3504, B*3505, B*3506, B*3507, B*3508, B*5101, B*5102, B*5103, B*5104, B*5105, B*5301, B*5401, B*5501, B*5502, B*5601, B*5602, B*6701, and B*7801 (see, e.g., Sidney, et al., J. Immunol. 154:247, 1995; Barber, et al., Curr. Biol. 5:179, 1995; Hill, et al., Nature 360:434, 1992; Rammensee, et al., Immunogenetics 41:178, 1995 for reviews of relevant data). Other allele-specific HLA molecules predicted to be members of the B7 supertype are shown in Table 4. As explained in detail below, peptide binding to each of the individual allele-specific HLA proteins can be modulated by substitutions at the primary and/or secondary anchor positions of the peptide, preferably choosing respective residues specified for the supermotif.

HLA-B27 supermotif. The HLA-B27 supermotif is characterized by the presence in peptide ligands of a positively charged (R, H, or K) residue as a primary anchor at position 2, and a hydrophobic (F, Y, L, W, M, L A, or V) residue as a primary anchor at the C-terminal position of the epitope (see, e.g., Sidney and Sette, Immunogenetics, in press, 1999). Exemplary members of the corresponding family of HLA molecules that bind to the B27 supermotif (i.e., the B27 supertype) include at least B*1401, B*1402, B*1509, B*2702, B*2703, B*2704, B*2705, B*2706, B*3801, B*3901, B*3902, and B*7301. Other allele-specific HLA molecules predicted to be members of the B27 supertype are shown in Table 4. Peptide binding to each of the allele-specific HLA molecules can be modulated by substitutions at primary and/or secondary anchor positions, preferably choosing respective residues specified for the supermotif.

HLA-B44 supermotif. The HLA-B44 supermotif is characterized by the presence in peptide ligands of negatively charged (D or E) residues as a primary anchor in position 2, and hydrophobic residues (F, W, Y, L, I, M, V, or A) as a primary anchor at the C-terminal position of the epitope (see, e.g., Sidney et al., Immunol. Today 17:261, 1996). Exemplary members of the corresponding family of HLA molecules that bind to the B44 supermotif (i.e., the B44 supertype) include at least: B*1801, B*1802, B*3701, B*4001, B*4002, B*4006, B*4402, B*4403, and B*4006. Peptide binding to each of the allele-specific HLA molecules can be modulated by substitutions at primary and/or secondary anchor positions; preferably choosing respective residues specified for the supermotif.

HLA-B58 supermotif. The HLA-B58 supermotif is characterized by the presence in peptide ligands of a small aliphatic residue (A, S, or T) as a primary anchor residue at position 2, and an aromatic or hydrophobic residue (F, W, Y, L, I, V, M, or A) as a primary anchor residue at the C-terminal position of the epitope (see, e.g., Sidney and Sette, Immunogenetics, in press, 1999 for reviews of relevant data). Exemplary members of the corresponding family of HLA molecules that bind to the B58 supermotif (i.e., the B58 supertype) include at least: B*1516, B*1517, B*5701, B*5702, and B*5801. Other allele-specific HLA molecules predicted to be members of the B58 supertype are shown in Table 4. Peptide binding to each of the allele-specific HLA molecules can be modulated by substitutions at primary and/or secondary anchor positions, preferably choosing respective residues specified for the supermotif.

HLA-B62 supermotif. The HLA-B62 supermotif is characterized by the presence in peptide ligands of the polar aliphatic residue Q or a hydrophobic aliphatic residue (L, V, M, I, or P) as a primary anchor in position 2, and a hydrophobic residue (F, W, Y, M, I, V, L, or A) as a primary anchor at the C-terminal position of the epitope (see, e.g., Sidney and Sette, Immunogenetics, in press, 1999). Exemplary members of the corresponding family of HLA molecules that bind to the B62 supermotif (i.e., the B62 supertype) include at least: B*1501, B*1502, B*1513, and B5201. Other allele-specific HLA molecules predicted to be members of the B62 supertype are shown in Table 4. Peptide binding to each of the allele-specific HLA molecules can be modulated by substitutions at primary and/or secondary anchor positions, preferably choosing respective residues specified for the supermotif.

HLA-A1 motif. The HLA-A1 motif is characterized by the presence in peptide ligands of T, S, or M as a primary anchor residue at position 2 and the presence of Y as a primary anchor residue at the C-terminal position of the epitope. An alternative allele-specific A1 motif is characterized by a primary anchor residue at position 3 rather than position 2. This motif is characterized by the presence of D, E, A, or S as a primary anchor residue in position 3, and a Y as a primary anchor residue at the C-terminal position of the epitope (see, e.g., DiBrino et al., J. Immunol., 152:620, 1994; Kondo et al., Immunogenetics 45:249, 1997; and Kubo et al., J. Immunol. 152:3913, 1994 for reviews of relevant data). Peptide binding to HLA A1 can be modulated by substitutions at primary and/or secondary anchor positions, preferably choosing respective residues specified for the motif.

Those epitopes comprising T, S, or M at position 2 and Y at the C-terminal position are also HLA-A1 supermotif-bearing peptide epitopes, as these residues are a subset of the A1 supermotif primary anchors.

HLA-A*0201 motif. An HLA-A2*0201 motif was determined to be characterized by the presence in peptide ligands of L or M as a primary anchor residue in position 2, and L or V as a primary anchor residue at the C-terminal position of a 9-residue peptide (see, e.g., Falk et al., Nature 351:290-296, 1991) and was further found to comprise an I at position 2 and I or A at the C-terminal position of a nine amino acid peptide (see, e.g., Hunt et al., Science 255:1261-1263, Mar. 6, 1992; Parker et al., J. Immunol. 149:3580-3587, 1992). The A*0201 allele-specific motif has also been defined by the present inventors to additionally comprise V, A, T, or Q as a primary anchor residue at position 2, and M or T as a primary anchor residue at the C-terminal position of the epitope (see, e.g., Kast et al., J. Immunol. 152:3904-3912, 1994). Thus, the HLA-A*0201 motif comprises peptide ligands with L, I, V, M, A, T, or Q as primary anchor residues at position 2 and L, I, V, M, A, or T as a primary anchor residue at the C-terminal position of the epitope. The preferred and tolerated residues that characterize the primary anchor positions of the HLA-A*0201 motif are identical to the residues describing the A2 supermotif (For reviews of relevant data, see, e.g., Del Guercio et al., J. Immunol. 154:685-693, 1995; Ruppert et al., Cell 74:929-937, 1993; Sidney et al., Immunol. Today 17:261-266, 1996; Sette and Sidney, Curr. Opin. in Immunol. 10:478-482, 1998). Secondary anchor residues that characterize the A*0201 motif have additionally been defined (see, e.g., Ruppert et al., Cell 74:929-937, 1993). These are shown in Table 3. Peptide binding to HLA-A*0201 molecules can be modulated by substitutions at primary and/or secondary anchor positions, preferably choosing respective residues specified for the motif.

HLA-A3 motif. The HLA-A3 motif is characterized by the presence in peptide ligands of L, M, V, I, S, A, T, F, C, G, or D as a primary anchor residue at position 2, and the presence of K, Y, R, H, F, or A as a primary anchor residue at the C-terminal position of the epitope (see, e.g., DiBrino et al., Proc. Natl. Acad. Sci USA 90:1508, 1993; and Kubo et al., J. Immunol. 152:3913-3924, 1994). Peptide binding to HLA-A3 can be modulated by substitutions at primary and/or secondary anchor positions, preferably choosing respective residues specified for the motif.

The A3 supermotif primary anchor residues comprise a subset of the A3- and A11-allele specific motif primary anchor residues.

HLA-A11 motif. The HLA-A11 motif is characterized by the presence in peptide ligands of V, T, M, L, I, S, A, G, N, C, D, or F as a primary anchor residue in position 2, and K, R, Y, or H as a primary anchor residue at the C-terminal position of the epitope (see, e.g., Zhang et al., Proc. Natl. Acad. Sci USA 90:2217-2221, 1993; and Kubo et al., J. Immunol. 152:3913-3924, 1994). Peptide binding to HLA-A11 can be modulated by substitutions at primary and/or secondary anchor positions, preferably choosing respective residues specified for the motif.

There is extensive overlap between the A3 and A11 motif primary anchor specificities.

HLA-A24 motif. The HLA-A24 motif is characterized by the presence in peptide ligands of Y, F, W, or M as a primary anchor residue in position 2, and F, L, I, or W as a primary anchor residue at the C-terminal position of the epitope (see, e.g., Kondo et al., J. Immunol. 155:4307-4312, 1995; and Kubo et al., J. Immunol. 152:3913-3924, 1994). Peptide binding to HLA-A24 molecules can be modulated by substitutions at primary and/or secondary anchor positions; preferably choosing respective residues specified for the motif.

The primary anchor residues characterizing the A24 allele-specific motif comprise a subset of the A24 supermotif primary anchor residues.

Computer or Manual Screening. Peptides bearing HLA Class I or Class II supermotifs or motifs may be identified by computer searches or manually, e.g., as follows. In utilizing computer screening to identify peptide epitopes, a protein sequence or translated sequence may be analyzed using software developed to search for motifs, for example the “FINDPATTERNS’ program (Devereux, et al. Nucl. Acids Res. 12:387-395, 1984) or MotifSearch 1.4 software program (D. Brown, San Diego, Calif.) to identify potential peptide sequences containing appropriate HLA binding motifs. The identified peptides can be scored using customized polynomial algorithms to predict their capacity to bind specific HLA class I or class II alleles. As appreciated by one of ordinary skill in the art, a large array of computer programming software and hardware options are available in the relevant art which can be employed to implement the motifs in order to evaluate (e.g., without limitation, to identify epitopes, identify epitope concentration per peptide length, or to generate analogs) known or unknown peptide sequences.

Translated antigen protein sequences may be analyzed using a text string search software program, e.g., MotifSearch 1.4 (D. Brown, San Diego) to identify potential peptide sequences containing appropriate HLA binding motifs; alternative programs are readily produced in accordance with information in the art in view of the motif/supermotif disclosure herein. Furthermore, such calculations can be made mentally.

Identified supermotif or motif sequences may be scored using polynomial algorithms to predict their capacity to bind to specific HLA-Class I or Class II molecules. These polynomial algorithms take into account both extended and refined motifs (that is, to account for the impact of different amino acids at different positions), and are essentially based on the premise that the overall affinity (or ΔG) of peptide-HLA molecule interactions can be approximated as a linear polynomial function of the type:
“ΔG”=a₁ᵢ×a₂ᵢ×a₃ᵢ . . . ×aₙᵢ
where aⱼᵢ is a coefficient which represents the effect of the presence of a given amino acid (j) at a given position (i) along the sequence of a peptide of n amino acids. The crucial assumption of this method is that the effects at each position are essentially independent of each other (i.e., independent binding of individual side-chains). When residue j occurs at position i in the peptide, it is assumed to contribute a constant amount jᵢ to the free energy of binding of the peptide irrespective of the sequence of the rest of the peptide. This assumption is justified by studies from our laboratories that demonstrated that peptides are bound to MHC and recognized by T cells in essentially an extended conformation (data omitted herein).

The method of derivation of specific algorithm coefficients has been described in Gulukota et al., J. Mol. Biol. 267:1258-126, 1997; (see also Sidney et al., Human Immunol. 45:79-93, 1996; and Southwood et al., J. Immunol. 160:3363-3373, 1998). Briefly, for all i positions, anchor and non-anchor alike, the geometric mean of the average relative binding (ARB) of all peptides carrying j is calculated relative to the remainder of the group, and used as the estimate of jᵢ. For Class II peptides, if multiple alignments are possible, only the highest scoring alignment is utilized, following an iterative procedure. To calculate an algorithm score of a given peptide in a test set, the ARB values corresponding to the sequence of the peptide are multiplied. If this product exceeds a chosen threshold, the peptide is predicted to bind. Appropriate thresholds are chosen as a function of the degree of stringency of prediction desired.

Additional methods to identify preferred peptide sequences, which also make use of specific motifs, include the use of neural networks and molecular modeling programs (see, e.g., Milik et al., Nature Biotechnology 16:753, 1998; Altuvia et al., Hum. Immunol. 58:1, 1997; Altuvia et al, J. Mol. Biol. 249:244, 1995; Buus, S. Curr. Opin. Immunol. 11:209-213, 1999; Brusic, V. et al., Bioinformatics 14:121-130, 1998; Parker et al., J. Immunol. 152:163, 1993; Meister et al., Vaccine 13:581, 1995; Hammer et al., J. Exp. Med. 180:2353, 1994; Sturniolo et al., Nature Biotechnol. 17:555 1999).

Conserved, Semi-conserved, and Non-conserved Non-anchor Residues. The determination of non-anchor residues as being conserved (conservative) or semi-conserved (semi-conservative) or non-conserved (non-conservative) in comparison to the non-anchor poitions of from one to all of the remaining variant(s) is defined by as follows, the results of which are summarized in Table 5.

Table 5 shows the similarity assignments between any given amino acid pair so that a given amino acid substitution could be characterized as being a (conservative) or semi-conserved (semi-conservative) or non-conserved (non-conservative) residue.

The degree of similarity between amino acid pairs was quantified by averaging, for each amino acid pair, the rank coefficient scores for PAM250, hydrophobicity, and side chain volume as described below. Based on the average values of these composite rankings, Table 5 shows each pair to be conserved, semi-conserved or non-conserved.

The Dayhoff PAM250 score (Dayhoff, M. O., et al., Atlas of Protein Sequence and Structure, Vol. 5, suppl.3. (1978) M. O. Dayhoff, ed. National Biomedical Research Foundation, Washington D.C., p. 345; Creighton, T. E., Proteins: structures and molecular properties (1993) (2nd edition) W.H. Freeman and Company, NY; http://prowl.rockefeller.edu/aainfo/pam250.html) is a commonly utilized protein alignment scoring matrix which measures the percentage of acceptable point mutations (PAM) within a defined time frame. The frequencies of these mutations are different from what would be expected from the probability of random mutations, and presumably reflect a bias due to the degree of physical and chemical similarity of the amino acid pair involved in the substitution. To obtain a score of amino acid similarity that could be standardized with other measures of similarity, the PAM250 scores were converted to a rank value, where 1 indicates the highest probability of being an accepted mutation.

The most commonly utilized scales to represent the relative hydrophobicity of the 20 naturally occurring amino acids (Cornette, J., et al., J. Mol. Biol. (1987) 195:659) are those developed on the basis of experimental data by Kyte and Doolittle (Kyte, J. and R. F. Doolittle, J. Mol. Biol. (1982) 157:105), and by Fauchere and Pliska (Fauchere, J. and V. Pliska, Eur. J. Med. Chem. (1983) 18:369). The Kyte/Doolittle scale measures the H₂O/organic solvent partition of individual amino acids. Because it considers the position of amino acids in folded proteins, it may most accurately reflect native hydrophobicity in the context of proteins. The Fauchere/Pliska scale measures the octanol/H₂O partitioning of N-acetyl amino acid amides, and most accurately reflects hydrophobicity in the context of denatured proteins and/or small synthetic peptides. To obtain scores for hydrophobicity, each amino acid residue was ranked on both the Kyte/Doolittle and Fauchere/Pliska hydrophobicity scales. An average rank between the two scales was calculated and the average difference in hydrophobicity for each pair was calculated.

Finally, for calculating amino acid side-chain volume, the partial volume in solution obtained by noting the increase in volume of water after adding either one molecule or one gram of amino acid residue was considered (Zamyatnin, A. A., Ann. Rev. Biophys. Bioeng. (1984) 13:145; Zamyatnin, A. A., Prog. Biophys. Mol. Biol. (1972) 24:107). The absolute difference in the partial volume of each possible pairing of the 20 naturally occurring amino acids was calculated and ranked, where 1 indicated residues with the most similar volumes, and 20 the most dissimilar.

Thus, by consulting Table 5, one can determine whether a residue in a variant is considered to be conserved, semi-conserved, or non-conserved in comparison to a residue in another variant(s). The residue of the parent variant (randomly or otherwise chosen variant) is shown across the top of Table 5, and the residue of the variant(s) it is compared with is shown below the parent residue.

As shown in Table 5, each of the amino acids shown across the top of the table bears a numerically defined relationship to the remaining 19 genetically encoded amino acids. The lower the index, the higher the conservation; the same amino acid will have a similarity assignment of 1.0; maximally different amino acids will have similarity assignments approaching 20. Using the method set forth above, amino acids which are not gene-encoded can also be assigned similarity indices and can be classified with respect to any natively occurring amino acid as conserved (conservative) or semi-conserved (semi-conservative) or non-conserved (non-conservative).

Variant Peptide Epitopes

In some embodiments, the invention is directed to an isolated peptide comprising or consisting of a variant. In some embodiments, the invention is directed to an isolated polynucleotide encoding such a peptide.

The isolated variants of the invention are all class I binding peptides, i.e., CTL peptides. In particular, the variants of the invention comprise a motif or supermotif, as described above. Variants of the invention are those set forth in Tables 6-9 and FIGS. 1A-4 (SEQ ID Nos:______). Variants of the invention may be referred to herein as “variants” and “variant peptide epitopes” or referred to by Table or referred to by SEQ ID NO. Other peptide epitopes are referred to herein as CTL epitopes or CTL peptides and HTL epitopes or HTL peptides.

Peptides and Polynucleotides. In some embodiments, the invention is directed to an isolated peptide comprising or consisting of a variant, wherein the variant consists of a sequence selected from those in Tables 6-9 and FIGS. 1A-4 (SEQ ID Nos:______).

Peptides of the invention may be fusion proteins of variant(s) to CTL epitope(s), and/or HTL epitope(s), and/or linker(s), and/or spacer(s), and/or carrier(s), and/or additional amino acid(s), and/or may comprise or consist of homopolymers of a variant or heteropolymers of more than one variant, as is described in detail below.

Peptides which comprise a variant of the invention may comprise or consist of a fragment of an antigen (“fragment” or “antigenic fragment”), wherein the fragment comprises a variant. The fragment may be a portion of any antigen of an infectious agent, e.g., the sequences in Tables 11-22 (SEQ ID Nos:______, respectively). The variant of the invention may be within the fragment or may be linked, directly or indirectly, to the fragment.

The fragment may comprise or consist of a region of a native antigen that contains a high concentration of class I and/or class II epitopes, preferably it contains the greatest number of epitopes per amino acid length. Such epitopes can be present in a frame-shifted manner, e.g. a 10 amino acid long peptide could contain two 9 amino acid long epitopes and one 10 amino acid long epitope.

The fragment may be less than or equal to 600 amino acids, less than or equal to 500 amino acids, less than or equal to 400 amino acids, less than or equal to 250 amino acids, less than or equal to 100 amino acids, less than or equal to 85 amino acids, less than or equal to 75 amino acids, less than or equal to 65 amino acids, or less than or equal to 50 amino acids in length. In certain embodiments, a fragment is less than 101 amino acids in length, in any increment down to 5 amino acids in length. For example, the fragment may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length. Fragments of full length antigens may be fragments from about residue 1-20, 21-40, 41-60, 61-80, 81-100, 101-120, 121-140, 141-160, 161-180, 181-200, 201-220, 221-240, 241-260, 261-280, 281-300, 301-320, 321-340, 341-360, 361-380, 381-400, 401-420, 421-440, 441-460, 461-480, 481-500, 501-520, 521-540, 541-560, 561-580, 581-600, 601-620, 621-680, 681-700, 701-720, 721-740, 741-780, 781-800, 801-820, 821-840, 841-860, 861-880, 881-900, 901-920, 921-940, 941-960, 961-980, 981 to the C-terminus of the antigen.

Peptides which comprise a variant of the invention may be a fusion protein comprising one or more amino acid residues in addition to the variant or fragment. Fusion proteins include homopolymers and heteropolymers, as described below.

In some embodiments, the peptide comprises or consists of multiple variants, e.g., 2, 3, 4, 5, 6, 7, 8, or 9 variants of the invention. In some embodiments, the peptide comprises at least 1, at least 2, at least 3, at least 4, at least 5, at least 6, at least 7, or at least 8 variants of the invention.

The peptide may also be a homopolymer of one variant or the peptide may be a heteropolymer which contains at least two different variants. Polymers have the advantage of increased probability for immunological reaction and, where different variants are used to make up the polymer, the ability to induce antibodies and/or T cells that react with different antigenic determinants of the antigen(s) targeted for an immune response.

A homopolymer may comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, or 150 copies of the same variant.

A heteropolymer may comprise one or more copies of an individual variant and one or more copies of one or more different variants of the invention. The variants that form a heteropolymer may all be from the same antigen, e.g., may be from any of those in Tables 11-22 (SEQ ID NOS:______) or other antigens herein or known in the art, or may be from different antigens, preferably from infectious agents. Combinations of variants that may form a heteropolymer include, for example, Gag 545 variants EPLTSLKSLF (SEQ ID NO:______) and YPLASLKSLF (SEQ ID NO:______), or combinations of peptides from different tables in Tables 6-9 and/or FIGS. 1A-4 or those combinations in Tables 23-28. Heteropolymers may contain multiple copies of one or more variants.

Thus, peptides of the invention such as heteropolymers may comprise a first variant and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 other (different) variants.

In some embodiments, the peptide comprising a variant may also comprise a number of CTL and/or HTL epitopes, e.g., 1,2,3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 CTL and/or HTL epitopes.

The CTL and/or HTL epitope and the variant of the invention may be from the same antigen of an infectious agent or from different antigens. Thus, for example, if the variant is from HIV pol, the CTL peptide and/or HTL peptide may also be from HIV pol. Alternatively, if the variant is from HIV pol, the CTL peptide and/or HTL peptide may be from another antigen such as HIV env or HIV vpr. As another example, if the variant is from HBV E6, the CTL peptide and/or HTL peptide may be from HBV E7. The CTL and/or HTL epitope and the variant of the invention may be from the same infectious agent or different infectious agents. Thus, for example, the variant may be from HIV, and the CTL and/or HTL epitope may be from HIV or may be from another infectious agent sush such as HBV, HCV, HPV, or Plasmodium falciparum.

The CTL peptide and/or HTL peptide may be from other antigens including hepatitis B core and surface antigens (HBVc, HBVs), hepatitis C antigens, Epstein-Barr virus antigens, human immunodeficiency virus (HIV) antigens and human papilloma virus (HPV) antigens (in particular anitgens from HPV-16, HPV-18, HPV-31, HPV-33, HPV-45, HPV-52, HPV-56 and HPV-58, Mycobacterium tuberculosis and Chlamydia. Examples of suitable fungal antigens include those derived from Candida albicans, Cryptococcus neoformans, Coccidoides spp., Histoplasma spp, and Aspergillus fumigatis. Examples of suitable protozoan parasitic antigens include those derived from Plasmodium spp., including P. falciparum, Trypanosoma spp., Schistosoma spp., Leishmania spp and the like.

Alternatively, the CTL peptide and/or HTL peptide may be from tumor-associated antigens such as but not limited to, melanoma antigens MAGE-1, MAGE-2, MAGE-3, MAGE-11, MAGE-A10, as well as BAGE, GAGE, RAGE, MAGE-C1, LAGE-1, CAG-3, DAM, MUC1, MUC2, MUC18, NY-ESO-1, MUM-1, CDK4, BRCA2, NY-LU-1, NY-LU-7, NY-LU-12, CASP8, RAS, KIAA-2-5, SCCs, p53, p73, CEA, HER2/neu, Melan-A, gp100, tyrosinase, TRP2, gp75/TRP1, kallikrein, prostate-specific membrane antigen (PSM), prostatic acid phosphatase (PAP), prostate-specific antigen (PSA), PT1-1, ∃-catenin, PRAME, Telomerase, FAK, cyclin D1 protein, NOEY2, EGF-R, SART-1, CAPB, HPVE7, p15, Folate receptor CDC27, PAGE-1, and PAGE-4.

Examples of CTL peptides and HTL peptides are disclosed in WO 01/42270, published 14 Jun. 2001; WO 01/41788, published 14 Jun. 2001; WO 01/42270, published 14 Jun. 2001; WO 01/45728, published 28 Jun. 2001; and WO 01/41787, published 14 Jun. 2001.

The HTL peptide may comprise a “loosely HLA-restricted” or “promiscuous” sequence. Examples of amino acid sequences that are promiscuous include sequences from antigens such as tetanus toxoid at positions 830-843 (QYIKANSKFIGITE; SEQ ID NO: 627), Plasmodium falciparum CS protein at positions 378-398 (DIEKKIAKMEKASSVFNVVNS; SEQ ID NO: 628), and Streptococcus 18 kD protein at positions 116-131 (GAVDSILGGVATYGAA; SEQ ID NO: 629). Other examples include peptides bearing a DR 1-4-7 supermotif, or either of the DR3 motifs.

The HTL peptide may comprise a synthetic peptide such as a Pan-DR-binding epitope (e.g., a PADRE® peptide, Epimmune Inc., San Diego, Calif., described, for example, in U.S. Pat. No. 5,736,142), for example, having the formula aKXVAAZTLKAAa, where “X” is either cyclohexylalanine, phenylalanine, or tyrosine; “Z” is either tryptophan, tyrosine, histidine or asparagine; and “a” is either D-alanine or L-alanine (SEQ ID NO: 746). Certain pan-DR binding epitopes comprise all “L” natural amino acids; these molecules can be provided as peptides or in the form of nucleic acids that encode the peptide. See also, U.S. Pat. Nos. 5,679,640 and 6,413,935.

The peptide comprising a variant may comprise additional amino acid(s). Such additional amino acids may be Ala, Arg, Asn, Asp, Cys, Gln, Gly, Glu, His, Ile, Leu, Lys, Met, Phe, Pro, Ser, Thr, Tyr, Trp, Val, amino acid mimetics, and other unnatural amino acids such as those described below. Additional amino acids may provide for ease of linking peptides one to another, for linking variants to one another, for linking variants to CTL and/or HTL epitopes, for coupling to a carrier support or larger peptide, for modifying the physical or chemical properties of the peptide or oligopeptide, or the like. Amino acids such as Ala, Arg, Asn, Asp, Cys, Gln, Gly, Glu, His, Ile, Leu, Lys, Met, Phe, Pro, Ser, Thr, Tyr, Trp, or Val, or the like, can be introduced at the C- and/or N-terminus of the peptide and/or can be introduced internally.

The peptide comprising a variant may comprise an amino acid spacer(s), which may be joined to the variants, CTL epitopes, HTL epitopes, carriers, etc. within a peptide or may be joined to the peptide at the N-and/or C-terminus. Thus, spacers may be at the N-terminus or C-terminus of peptide, or may be internal such that they link or join variants, CTL epitopes, HTL epitopes, carriers, additional amino acids, and/or antigenic fragments one to the other.

The spacer is typically comprised of one or more relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer may be composed of the same residues or may be composed of one or more different residues and thus may be a homo- or hetero-oligomer of spacer residues. Thus, the spacer may contain more than one Ala residue (poly-alanine) or more than one Gly residue (poly-glycine), or may contain both Ala and Gly residues, e.g., Gly, Gly-Gly-, Ser,Ser-Ser-, Gly-Ser-, Ser-Gly-, etc. When present, the spacer will usually be at least one or two residues, more usually three to six residues and sometimes 10 or more residues, e.g., 3, 4, 5, 6, 7, 8, 9, or 10, or even more residues. (Livingston, B. D. et al. Vaccine 19:4652-4660 (2000)).

Peptides comprising a variant may comprise carrier(s) such as those well known in the art, e.g., thyroglobulin, albumins such as human serum albumin, tetanus toxoid, polyamino acids such as poly L-lysine, poly L-glutamic acid, influenza virus proteins, hepatitis B virus core protein, and the like. (See Table 29).

In addition, the peptide comprising or consisting of a variant may be modified by terminal-NH₂ acylation, e.g., by alkanoyl (C₁-C₂₀) or thioglycolyl acetylation, terminal-carboxyl amidation, e.g., ammonia, methylamine, etc. In some instances these modifications may provide sites for linking to a support or other molecule.

The peptides in accordance with the invention can contain modifications such as but not limited to glycosylation, side chain oxidation, biotinylation, phosphorylation, addition of a surface active material, e.g. a lipid, or can be chemically modified, e.g., acetylation, etc. Moreover, bonds in the peptide can be other than peptide bonds, e.g., covalent bonds, ester or ether bonds, disulfide bonds, hydrogen bonds, ionic bonds, etc.

Peptides of the present invention may contain substitutions to modify a physical property (e.g., stability or solubility) of the resulting peptide. For example, peptides may be modified by the substitution of a cysteine (C) with α-amino butyric acid (“B”). Due to its chemical nature, cysteine has the propensity to form disulfide bridges and sufficiently alter the peptide structurally so as to reduce binding capacity. Substituting α-amino butyric acid for C not only alleviates this problem, but actually improves binding and crossbinding capability in certain instances. Substitution of cysteine with α-amino butyric acid may occur at any residue of a peptide, e.g., at either anchor or non-anchor positions of a variant within a peptide, or at other positions of a peptide.

The peptides comprising a variant can comprise amino acid mimetics or unnatural amino acids, e.g. D- or L-naphylalanine; D- or L-phenylglycine; D- or L-2-thieneylalanine; D- or L-1, -2, 3, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-ρ-fluorophenylalanine; D- or L-ρ-biphenylphenylalanine; D- or L-ρ-methoxybiphenylphenylalanine; D- or L-2-indole(alkyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g. thiazolyl, thiophenyl, pyrazolyl, benzimidazolyl, naphthyl, furanyl, pyrrolyl, and pyridyl aromatic rings. Modified peptides that have various amino acid mimetics or unnatural amino acids are particularly useful, as they tend to manifest increased stability in vivo. Such peptides may also possess improved shelf-life or manufacturing properties.

Peptide stability can be assayed in a number of ways. For instance, peptidases and various biological media, such as human plasma and serum, have been used to test stability. See, e.g., Verhoef, et al., Eur. J. Drug Metab. Pharmacokinetics 11:291 (1986). Half-life of the peptides of the present invention is conveniently determined using a 25% human serum (v/v) assay. The protocol is generally as follows: Pooled human serum (Type AB, non-heat inactivated) is delipidated by centrifugation before use. The serum is then diluted to 25% with RPMI-1640 or another suitable tissue culture medium. At predetermined time intervals, a small amount of reaction solution is removed and added to either 6% aqueous trichloroacetic acid (TCA) or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

As indicated above, the peptides in accordance with the invention can be a variety of lengths, and either in their neutral (uncharged) forms or in forms which are salts. The peptides in accordance with the invention can contain modifications such as glycosylation, side chain oxidation, or phosphorylation, generally subject to the condition that modifications do not destroy the biological activity of the peptides.

The peptides of the invention may be lyophylized, or may be in crystal form.

It is generally preferable that the variant peptide epitope be as small as possible while still maintaining substantially all of the immunologic activity of the native protein. When possible, it may be desirable to optimize HLA class I binding epitopes of the invention to a length of about 8 to about 13 amino acid residues, for example, 8, 9, 10, 11, 12 or 13, preferably 8 to 11 or 9 to 10. It is to be appreciated that one or more epitopes in this size range can be comprised by a longer peptide (see the Definition Section for the term “epitope” for further discussion of peptide length). HLA class II binding epitopes are preferably optimized to a length of about 6 to about 30 amino acids in length, e.g., 6, 7, 8, 9, 10, 11, 12, 13, 14, 15, 16, 17, 18, 19, 20, 21, 22, 23, 24, 25, 26, 27, 28, 29 or 30, preferably to between about 13 and about 20 residues, e.g., 13, 14, 15, 16, 17, 18, 19 or 20. Preferably, the epitopes are commensurate in size with endogenously processed pathogen-derived peptides or tumor cell peptides that are bound to the relevant HLA molecules. The identification and preparation of peptides of various lengths can be carried out using the techniques described herein.

Peptides in accordance with the invention can be prepared synthetically, by recombinant DNA technology or chemical synthesis, or can be isolated from natural sources such as native tumors or pathogenic organisms. Epitopes may be synthesized individually or joined directly or indirectly in a peptide. Although the peptide will preferably be substantially free of other naturally occurring host cell proteins and fragments thereof, in some embodiments the peptides may be synthetically conjugated to be joined to native fragments or particles.

The peptides of the invention can be prepared in a wide variety of ways. For relatively short sizes, the peptides can be synthesized in solution or on a solid support in accordance with conventional techniques. Various automatic, synthesizers are commercially available and can be used in accordance with known protocols. (See, for example, Stewart & Young, SOLID PHASE PEPTIDE SYNTHESIS, 2D. ED., Pierce Chemical Co., 1984). Further, individual peptides can be joined using chemical ligation to produce larger peptides that are still within the bounds of the invention.

Alternatively, recombinant DNA technology can be employed wherein a nucleotide sequence which encodes a peptide inserted into an expression vector, transformed or transfected into an appropriate host cell and cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989). Thus, recombinant peptides, which comprise or consist of one or more epitopes of the invention, can be used to present the appropriate T cell epitope.

Polynucleotides encoding each of the peptides above are also part of the invention. As appreciated by one of ordinary skill in the art, various nucleic acids will encode the same peptide due to the redundancy of the genetic code. Each of these nucleic acids falls within the scope of the present invention. This embodiment of the invention comprises DNA and RNA, and in certain embodiments a combination of DNA and RNA. It is to be appreciated that any polynucleotide that encodes a peptide in accordance with the invention falls within the scope of this invention.

The polynucleotides encoding peptides contemplated herein can be synthesized by chemical techniques, for example, the phosphotriester method of Matteucci, et al., J. Am. Chem. Soc. 103:3185 (1981). Polynucleotides encoding peptides comprising or consisting of a variant can be made simply by substituting the appropriate and desired nucleic acid base(s) for those that encode a related (e.g., analogous) epitope.

The polynucleotide, e.g. minigene (see below), may be produced by assembling oligonucleotides that encode the plus and minus strands of the polynucleotide, e.g. minigene. Overlapping oligonucleotides (15-100 bases long) may be synthesized, phosphorylated, purified and annealed under appropriate conditions using well known techniques. The ends of the oligonucleotides can be joined, for example, using T4 DNA ligase. A polynucleotide, e.g. minigene, encoding the peptide of the invention, can be cloned into a desired vector such as an expression vector. The coding sequence can then be provided with appropriate linkers and ligated into expression vectors commonly available in the art, and the vectors used to transform suitable hosts to produce the desired peptide such as a fusion protein.

A large number of such vectors and suitable host systems are known to those of skill in the art, and are commercially available. The following vectors are provided by way of example. Bacterial: pQE70, pQE60, pQE-9 (Qiagen), pBS, pD10, phagescript, psiX174, pBluescript SK, pbsks, pNH8A, pNH16a, pNH18A, pNH46A (Stratagene); ptrc99a, pKK223-3, pKK233-3, pDR540, pRIT5 (Pharmacia); pCR (Invitrogen). Eukaryotic: pWLNEO, pSV2CAT, pOG44, PXT1, pSG (Stratagene) pSVK3, pBPV, pMSG, pSVL (Pharmacia); p75.6 (valentis); pCEP (Invitrogen); pCEI (Epimmune). However, any other plasmid or vector can be used as long as it is replicable and viable in the host.

As representative examples of appropriate hosts, there can be mentioned: bacterial cells, such as E. coli, Bacillus subtilis, Salmonella typhimurium and various species within the genera Pseudomonas, Streptomyces, and Staphylococcus; fungal cells, such as yeast; insect cells such as Drosophila and Sf9; animal cells such as COS-7 lines of monkey kidney fibroblasts, described by Gluzman, Cell 23:175 (1981), and other cell lines capable of expressing a compatible vector, for example, the C127, 3T3, CHO, HeLa and BHK cell lines or Bowes melanoma; plant cells, etc. The selection of an appropriate host is deemed to be within the scope of those skilled in the art from the teachings herein.

Thus, the present invention is also directed to vectors, preferably expression vectors useful for the production of the peptides of the present invention, and to host cells comprising such vectors.

Host cells are genetically engineered (transduced or transformed or transfected) with the vectors of this invention which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the polynucletides. The culture conditions, such as temperature, pH and the like, are those previously used with the host cell selected for expression, and will be apparent to the ordinarily skilled artisan.

For expression of the peptides, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), ∀-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Yeast, insect or mammalian cell hosts may also be used, employing suitable vectors and control sequences. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. Such promoters may also be derived from viral sources, such as, e.g., human cytomegalovirus (CMV-IE promoter) or herpes simplex virus type-1 (HSV TK promoter). Nucleic acid sequences derived from the SV40 splice, and polyadenylation sites can be used to provide the required nontranscribed genetic elements.

Polynucleotides encoding peptides of the invention may also comprise a ubiquitination signal sequence, and/or a targeting sequence such as an endoplasmic reticulum (ER) signal sequence to facilitate movement of the resulting peptide into the endoplasmic reticulum.

Polynucleotides of the invention, e.g., minigenes, may be expressed in human cells. A human codon usage table can be used to guide the codon choice for each amino acid. Such polynucleotides preferably comprise spacer amino acid residues between variants, such as those described above, or may comprise naturally-occurring flanking sequences adjacent to the variants (and/or CTL and HTL epitopes).

The peptides of the invention can also be expressed by viral or bacterial vectors. Examples of expression vectors include attenuated viral hosts, such as vaccinia or fowlpox. As an example of this approach, vaccinia virus is used as a vector to express nucleotide sequences that encode the peptides of the invention. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the polypeptides of the invention, e.g. adeno and adeno-associated virus vectors, retroviral vectors, Salmonella typhi vectors, detoxified anthrax toxin vectors, and the like, will be apparent to those skilled in the art from the description herein. A preferred vector is Modified Vaccinia Ankara (MVA) (e.g., Bavarian Noridic (MVA-BN)).

Standard regulatory sequences well known to those of skill in the art are preferably included in the vector to ensure expression in the human target cells. Several vector elements are desirable: a promoter with a downstream cloning site for polynucleotide, e.g., minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences. A preferred promoter is the CMV-IE promoter.

Polynucleotides, e.g. minigenes, may comprise one or more synthetic or naturally-occurring introns in the transcribed region. The inclusion of mRNA stabilization sequences and sequences for replication in mammalian cells may also be considered for increasing polynucleotide, e.g. minigene, expression.

In addition, the polynucleotide, e.g. minigene, may comprise immunostimulatory sequences (ISSs or CpGs). These sequences may be included in the vector, outside the polynucleotide (e.g. minigene) coding sequence to enhance immunogenicity.

In some embodiments, a bi-cistronic expression vector which allows production of both the polynucleotide- (e.g. minigene-) encoded peptides of the invention and a second protein (e.g., one that modulates immunogenicity) can be used. Examples of proteins or polypeptides that, if co-expressed with peptides of the invention, can enhance an immune response include cytokines (e.g., IL-2, IL-12, GM-CSF), cytokine-inducing molecules (e.g., LeIF), costimulatory molecules, or pan-DR binding proteins (PADRE® molecules, Epimmune, San Diego, Calif.). Helper T cell (HTL) epitopes such as PADRE® molecules can be joined to intracellular targeting signals and expressed separately from expressed peptides of the invention. Specifically decreasing the immune response by co-expression of immunosuppressive molecules (e.g. TGF-β) may be beneficial in certain diseases.

Once an expression vector is selected, the polynucleotide, e.g. minigene, is cloned into the polylinker region downstream of the promoter. This plasmid is transformed into an appropriate bacterial strain, and DNA is prepared using standard techniques. The orientation and DNA sequence of the polynucleotide, e.g. minigene, as well as all other elements included in the vector, are confirmed using restriction mapping, DNA sequence analysis, and/or PCR analysis. Bacterial cells harboring the correct plasmid can be stored as cell banks.

Therapeutic/prophylactic quantities of DNA can be produced for example, by fermentation in E. coli, followed by purification. Aliquots from the working cell bank are used to inoculate growth medium, and are grown to saturation in shaker flasks or a bioreactor according to well known techniques. Plasmid DNA is purified using standard bioseparation technologies such as solid phase anion-exchange resins available, e.g., from QIAGEN, Inc. (Valencia, Calif.). If required, supercoiled DNA can be isolated from the open circular and linear forms using gel electrophoresis or other methods.

Purified polynucleotides, e.g. minigenes, can be prepared for injection using a variety of formulations. The simplest of these is reconstitution of lyophilized polynucleotide, e.g. DNA, in sterile phosphate-buffer saline (PBS). This approach, known as “naked DNA,” is currently being used for intramuscular (IM) administration in clinical trials. To maximize the immunotherapeutic effects of polynucleotide vaccines, alternative methods of formulating purified plasmid DNA may be used. A variety of such methods have been described, and new techniques may become available. Cationic lipids, glycolipids, and fusogenic liposomes can also be used in the formulation (see, e.g. WO 93/24640; Mannino & Gould-Fogerite, BioTechniques 6(7): 682 (1988); U.S. Pat. No. 5,279,833; WO 91/06309; and Felgner, et al., Proc. Natl. Acad. Sci. USA 84:7413 (1987). In addition, peptides and compounds referred to collectively as protective, interactive, non-condensing compounds (PINC) can also be complexed to purified plasmid DNA to influence variables such as stability, intramuscular dispersion, or trafficking to specific organs or cell types.

Known methods in the art can be used to enhance delivery and uptake of a polynucleotide in vivo. For example, the polynucleotide can be complexed to polyvinylpyrrolidone (PVP), to prolong the localized bioavailability of the polynucleotide, thereby enhancing uptake of the polynucleotide by the organisum (see e.g., U.S. Pat. No. 6,040,295; EP 0 465 529; WO 98/17814). PVP is a polyamide that is known to form complexes with a wide variety of substances, and is chemically and physiologically inert.

Target cell sensitization can be used as a functional assay of the expression and HLA class I presentation of polynucleotide- (e.g. minigene-) encoded peptides. For example, the polynucleotide, e.g. plasmid DNA, is introduced into a mammalian cell line that is a suitable target for standard CTL chromium release assays. The transfection method used will be dependent on the final formulation. For example, electroporation can be used for “naked” DNA, whereas cationic lipids or PVP-formulated DNA allow direct in vitro transfection. A plasmid expressing green fluorescent protein (GFP) can be co-transfected to allow enrichment of transfected cells using fluorescence activated cell sorting (FACS). The transfected cells are then chromium-51 (⁵¹Cr) labeled and used as targets for epitope-specific CTLs. Cytolysis of the target cells, detected by ⁵¹Cr release, indicates both production and HLA presentation of, polynucleotide-, e.g. minigene-, encoded variants of the invention, or peptides comprising them. Expression of HTL epitopes may be evaluated in an analogous manner using assays to assess HTL activity.

In vivo immunogenicity is a second approach for functional testing of polynucleotides, e.g. minigenes. Transgenic mice expressing appropriate human HLA proteins are immunized with the polynucleotide, e.g. DNA, product. The dose and route of administration are formulation dependent (e.g., IM for polynucleotide (e.g. naked DNA or PVP-formulated DNA) in PBS, intraperitoneal (IP) for lipid-complexed polynucleotide (e.g., DNA)). Eleven to twenty-one days after immunization, splenocytes are harvested and restimulated for one week in the presence of polynucleotides encoding each peptide being tested. Thereafter, for peptides comprising or consisting of variants, standard assays are conducted to determine if there is cytolysis of peptide-loaded, ⁵¹Cr-labeled target cells. Once again, lysis of target cells that were exposed to variants corresponding to those encoded by the polynucleotide (e.g. minigene) demonstrates polynucleotide (e.g., DNA) vaccine function and induction of CTLs. Immunogenicity of HTL epitopes is evaluated in transgenic mice in an analogous manner.

Alternatively, the nucleic acids can be administered using ballistic delivery as described, for instance, in U.S. Pat. No. 5,204,253. Using this technique, particles comprised solely of a polynucleotide such as DNA are administered. In a further alternative embodiment for ballistic delivery, polynucleotides such as DNA can be adhered to particles, such as gold particles.

The use of polynucleotides such as multi-epitope minigenes is described herein and in, e.g. co-pending application U.S. Ser. No. 09/311,784; Ishioka et al., J. Immunol. 162:3915-3925, 1999; An, L. and Whitton, J. L., J. Virol. 71:2292, 1997; Thomson, S. A. et al., J. Immunol. 157:822, 1996; Whitton, J. L. et al., J. Virol. 67:348, 1993; Hanke, R. et al., Vaccine 16:426, 1998. For example, a polynucleotide such as a multi-epitope DNA plasmid can be engineered which encodes an epitope derived from multiple regions of a infectious agent (e.g., p53, HER2/nev, MAGE-2/3, or CEA), a pan-DR binding peptide such as the PADRE® universal helper T cell epitope, and an endoplasmic reticulum-translocating signal sequence. As descibed in the sections above, a peptide/polynucleotide may also comprise/encode epitopes that are derived from other infectious agents.

Thus, the invention includes peptides as described herein, polynucleotides encoding each of said peptides, as well as compositions comprising the peptides and polynucleotides, and includes methods for producing and methods of using the peptides, polynucleotides, and compositions, as further described below.

Compositions. In other embodiments, the invention is directed to a composition comprising one or more peptides and/or polynucleotides of the invention and optionally another component(s).

In some embodiments, the composition comprises or consists of multiple peptides, e.g., 2, 3, 4, 5, 6, 7, 8, or 9 peptides of the invention. In some embodiments, the composition comprises at least 2, at least 3, at least 4, at least 5, at least 6, at least 7, or at least 8 peptides of the invention. Combinations of peptides include, for example, a peptide comprising or alternatively consisting of the Gag 545 variant EPLTSLKSLF (SEQ ID NO:______) and a peptide comprising or alternatively consisting of the Gag 545 variant YPLASLKSLF (SEQ ID NO:______), or combinations of peptides from different tables in Tables 6-9 and/or FIGS. 1A-4.

Compositions of the invention may comprise polynucleotides encoding the above peptides and/or combinations of peptides.

The composition can comprise at least 2, at least 3, at least 4, at least 5, at least 6, at least 7, or at least 8 peptides and/or polynucleotides selected from those described above or below. At least one of the one or more peptides can be a heteropolymer or a homopolymer. Additionally, the composition can comprise a CTL and/or HTL epitope, which can be derived from a tumor-associated antigen. The additional epitope can also be a PanDR binding molecule, (e.g., a PADRE® universal helper T cell epitope).

Optional components include excipients, diluents, proteins such as peptides comprising a CTL epitope, and/or an HTL epitope such as a pan-DR binding peptide (e.g., a PADRE® universal helper T cell epitope), and/or a carrier, polynucleotides encoding such proteins, lipids, or liposomes, as well as other components described herein. There are numerous embodiments of compositions in accordance with the invention, such as a cocktail of one or more peptides and/or polynucleotides (e.g., minigenes); a cocktail of one or more peptides and/or polynucleotides (e.g., minigenes) and one or more CTL and/or HTL epitopes.

Compositions may comprise one or more peptides (and/or polynucleotides such as minigenes) of the invention, along with one or more other components as described above and herein. “One or more” refers to any whole unit integer from 1-150, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, or 150 peptides, polynucleotides, or other components.

Compositions of the invention may be, for example, polynucleotides or polypeptides of the invention combined with or complexed to cationic lipid formulations; lipopeptides (e.g., Vitiello, A. et al., J. Clin. Invest. 95:341, 1995), encapsulated e.g., in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995); peptide compositions contained in immune stimulating complexes (ISCOMS) (see, e.g., Takahashi et al., Nature 344:873-875, 1990; Hu et al., Clin Exp Immunol. 113:235-243, 1998); multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996); viral, bacterial, or, fungal delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990); particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995); adjuvants (e.g., incomplete Freund's adjuvant) (Warren, H. S., Vogel, F. R., and Chedid, L. A. Annu. Rev. Immunol. 4:369, 1986; Gupta, R. K. et al., Vaccine 11:293, 1993); liposomes (Reddy, R. et al., J. Immunol. 148:1585, 1992; Rock, K. L., Immunol. Today 17:131, 1996); or, particle-absorbed cDNA or other polynucleotides of the invention (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993), etc. Toxin-targeted delivery technologies, also known as receptor mediated targeting, such as those of Avant Immunotherapeutics, Inc. (Needham, Mass.) or attached to a stress protein, e.g., HSP 96 (Stressgen Biotechnologies Corp., Victoria, BC, Canada) can also be used.

Compositions of the invention comprise polynucleotide-mediated modalities. DNA or RNA encoding one or more of the peptides of the invention can be administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; and, WO 98/04720. Examples of DNA-based delivery technologies include “naked DNA”, facilitated (bupivicaine, polymers (e.g., PVP, PINC, etc.), peptide-mediated) delivery, cationic lipid complexes, and particle-mediated (“gene gun”) or pressure-mediated delivery (see, e.g., U.S. Pat. No. 5,922,687). Accordingly, peptides of the invention can be expressed by viral or bacterial vectors. Examples of expression vectors include attenuated viral hosts, such as Modified Vaccinia Ankara (MVA) (e.g., Bavarian Noridic), vaccinia or fowlpox. For example, vaccinia virus is used as a vector to express nucleotide sequences that encode the peptides of the invention. Upon introduction into an acutely or chronically infected host or into a non-infected host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the peptides of the invention, e.g. adeno and adeno-associated virus vectors, alpha virus vectors, retroviral vectors, Salmonella typhi vectors, detoxified anthrax toxin vectors, and the like, are apparent to those skilled in the art from the description herein.

In certain embodiments, components that induce T cell responses are combined with components that induce antibody responses to the target antigen of interest. A preferred embodiment of such a composition comprises class I and class II epitopes in accordance with the invention. Alternatively, a composition comprises a class I and/or class II epitope in accordance with the invention, along with a PADRE® molecule (Epimmune, San Diego, Calif.).

Compositions of the invention can comprise antigen presenting cells, such as dendritic cells. Antigen presenting cells, e.g., dendritic cells, may be transfected, e.g., with a polynucleotide such as a minigene construct in accordance with the invention, in order to elicit immune responses. The peptide can be bound to an HLA molecule on the antigen-resenting cell, whereby when an HLA-restricted cytotoxic T lymphocyte (CTL) is present, a receptor of the CTL binds to a complex of the HLA molecule and the peptide.

The compositions of the invention may also comprise antiviral drugs such as interferon-α, or immune adjuvants such as IL-12, GM-CSF, etc.

Compositions may comprise an HLA heavy chain, β₂-microglobulin, streptavidin, and/or biotin. The streptavidin may be fluorescently labeled. Compositions may comprise tetramers (see e.g., U.S. Pat. No. 5,635,363; Science 274:94-96 (1996)). A tetramer composition comprising an HLA heavy chain, β₂-microglobulin, streptavidin, and biotin. The streptavidin may be fluorescently labeled. Compositions may also comprise dimers. A dimer composition comprises as MHC molecule and an Ig molecule (see e.g., PNAS 95:7568-73 (1998)).

In some embodiments it may be desirable to include in the compositions of the invention at least one component which primes cytotoxic T lymphocytes. Lipids have been identified as agents capable of priming CTL in vivo against viral antigens. For example, palmitic acid residues can be attached to the ε-and α-amino groups of a lysine residue and then linked, e.g., via one or more linking residues such as Gly, Gly-Gly-, Ser, Ser-Ser, or the like, to an immunogenic peptide. The lipidated peptide can then be administered either directly in a micelle or particle, incorporated into a liposome, or emulsified in an adjuvant, e.g., incomplete Freund's adjuvant. A preferred composition comprises palmitic acid attached to ε- and α-amino groups of Lys, which is attached via linkage, e.g., Ser-Ser, to the amino terminus of the peptide.

As another example of lipid priming of CTL responses, E. coli lipoproteins, such as tripalmitoyl-S-glycerylcysteinlyseryl-serine (P₃CSS) can be used to prime virus specific CTL when covalently attached to an appropriate peptide (see, e.g., Deres, et al., Nature 342:561, 1989). Peptides of the invention can be coupled to P₃CSS, for example, and the lipopeptide administered to an individual to specifically prime a CTL response to the target antigen. Moreover, because the induction of neutralizing antibodies can also be primed with P₃CSS-conjugated epitopes, two such compositions can be combined to more effectively elicit both humoral and cell-mediated responses.

Another preferred embodiment is a composition comprising one or more peptides of the invention emulsified in IFA.

Compositions of the invention may also comprise CTL and/or HTL peptides. Such CTL and HTL peptides can be modified by the addition of amino acids to the termini of a peptide to provide for ease of linking peptides one to another, for coupling to a carrier support or larger peptide, for modifying the physical or chemical properties of the peptide or oligopeptide, or the like. Amino acids such as tyrosine, cysteine, lysine, glutamic or aspartic acid, or naturally or unnaturally occuring amino acid residues, can be introduced at the carboxyl- or amino-terminus of the peptide or oligopeptide, particularly class I peptides. However, it is to be noted that modification at the carboxyl terminus of a CTL epitope may, in some cases, alter binding characteristics of the peptide. In addition, the peptide or oligopeptide sequences can differ from the natural sequence by being modified by terminal-NH₂ acylation, e.g., by alkanoyl (C₁-C₂₀) or thioglycolyl acetylation, terminal-carboxyl amidation, e.g., ammonia, methylamine, etc. In some instances these modifications may provide sites for linking to a support or other molecule. CTL and HTL epitopes may comprise additional amino acids, such as those described above including spacers.

A further embodiment of a composition in accordance with the invention is an antigen presenting cell that comprises one or more peptides in accordance with the invention. The antigen presenting cell can be a “professional” antigen presenting cell, such as a dendritic cell. The antigen presenting cell can comprise the peptide of the invention by any means known or to be determined in the art. Such means include pulsing of dendritic cells with one or more individual peptides, by nucleic acid administration such as ballistic nucleic acid delivery or by other techniques in the art for administration of nucleic acids, including vector-based, e.g. viral vector, delivery of nucleic acids.

Compositions may comprise carriers. Carriers that can be used with compositions of the invention are well known in the art, and include, e.g., thyroglobulin, albumins such as human serum albumin, tetanus toxoid, polyamino acids such as poly L-lysine, poly L-glutamic acid, influenza virus proteins, hepatitis B virus core protein, and the like.

The compositions (e.g. pharmaceutical compositions) can contain a physiologically tolerable diluent such as water, or a saline solution, preferably phosphate buffered saline. Additionally, as disclosed herein, CTL responses can be primed by conjugating peptides of the invention to lipids, such as tripalmitoyl-S-glyceryl-cysteinyl-seryl-serine (P₃CSS).

Compositions of the invention may be pharmaceutically acceptable compositions. Pharmaceutical compositions preferably contain an immunologically effective amount of one or more peptides and/or polynucleotides of the invention, and optionally one or more other components which are pharmaceutically acceptable. A preferred composition comprises one or more peptides of the invention and IFA. A more preferred composition of the invention comprises one or more peptides of the invention, one or more peptides, and IFA.

Upon immunization with a peptide and/or polynucleotide and/or composition in accordance with the invention, via injection (e.g., SC, ID, IM), aerosol, oral, transdermal, transmucosal, intrapleural, intrathecal, or other suitable routes, the immune system of the host responds to the vaccine by an immune response comprising the production of antibodies, CTLs and/or HTLs specific for the desired antigen(s). Consequently, the host becomes at least partially immune to subsequent exposure to the infectious agent(s), or at least partially resistant to further development of infectious agent-bearing cells and thereby derives a prophylactic or therapeutic benefit.

Furthermore, the peptides, primers, and epitopes of the invention can be used in any desired immunization or administration regimen; e.g., as part of periodic vaccinations such as annual vaccinations as in the veterinary arts or as in periodic vaccinations as in the human medical arts, or as in a prime-boost regime wherein an inventive vector or recombinant is administered either before or after the administration of the same or of a different epitope of interest or recombinant or vector expressing such as a same or different epitope of interest (including an inventive recombinant or vector expressing such as a same or different epitope of interest), see, e.g., U.S. Pat. Nos. 5,997,878; 6,130,066; 6,180,398; 6,267,965; and 6,348,450. An useful viral vector of the present invention is Modified Vaccinia Ankara (MVA) (e.g. Bavarian Noridic (MVA-BN)).

Recent studies have indicated that a prime-boost protocol, whereby immunization with a poxvirus recombinant expressing a foreign gene product is followed by a boost using a purified subunit preparation form of that gene product, elicits an enhanced immune response relative to the response elicited with either product alone. Human volunteers immunized with a vaccinia recombinant expressing the HIV-1 envelope glycoprotein and boosted with purified HIV-1 envelope glycoprotein subunit preparation exhibit higher HIV-1 neutralizing antibody titers than individuals immunized with just the vaccinia recombinant or purified envelope glycoprotein alone (Graham et al., J. Infect. Dis., 167:533-537 (1993); Cooney et al., Proc. Natl. Acad. Sci. USA, 90:1882-1886 (1993)). Humans immunized with two injections of an ALVAC-HIV-1 env recombinant (vCP125) failed to develop BV specific antibodies. Boosting with purified rgp160 from a vaccinia virus recombinant resulted in detectable HIV-1 neutralizing antibodies. Furthermore, specific lymphocyte T cell proliferation to rgp160 was clearly increased by the boost with rgp160. Envelope specific cytotoxic lymphocyte activity was also detected with this vaccination regimen (Pialoux et al., AIDS Res. and Hum. Retroviruses, 11:272-381 (1995)). Macaques immunized with a vaccinia recombinant expressing the simian immunodeficiency virus (SIV) envelope glycoprotein and boosted with SIV envelope glycoprotein from a baculovirus recombinant are protected against SIV challenge (Hu et al., AID Res. and Hum. Retroviruses, 3:615-620 (1991); Hu et al., Science 255:456-459 (1992)). In the same fashion, purified HCMVgB protein can be used in prime-boost protocols with NYVAC or ALVAC-gB recombinants.

In certain embodiments, the polynucleotides are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA 84:74137416 (1987), which is herein incorporated by reference); mRNA (Malone et al., Proc. Natl. Acad. Sci. USA 86:60776081 (1989), which is herein incorporated by reference); and purified transcription factors (Debs et al., J. Biol. Chem. 265:1018910192 (1990), which is herein incorporated by reference), in functional form.

Cationic liposomes are readily available. For example, N-[12,3-dioleyloxy)-propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA 84:74137416 (1987)). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:74137417. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially available dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome nucleic acid complexes are prepared using methods well known in the art. See, e.g., Straubinger et al., Methods of Immunology 101:512527 (1983). For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta 394:483 (1975); Wilson et al, Cell 17:77 (1979)); ether injection (Deamer, D. and Bangham, A., Biochim. Biophys. Acta 443:629 (1976); Ostro et al., Biochem. Biophys. Res. Commun. 76:836 (1977); Fraley et al., Proc. Natl. Acad. Sci. USA 76:3348 (1979)); detergent dialysis (Enoch, H. and Strittmatter, P., Proc. Natl. Acad. Sci. USA 76:145 (1979)); and reversephase evaporation (REV) (Fraley et al., J. Biol. Chem. 255:10431 (1980); Szoka, F. and Papahadjopoulos, D., Proc. Natl. Acad. Sci. USA 75:145 (1978); SchaeferRidder et al., Science 215:166 (1982)).

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 reports on the injection of genetic material, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 provide cationic lipids for use in transfecting DNA into cells and mammals. U.S. Pat. Nos. 5,589,466, 5,693,622, 5,580,859, 5,703,055, and international publication no. WO 94/9469 provide methods for delivering DNA-cationic lipid complexes to mammals.

Binding Affinity of Variants for HLA Molecules

As indicated herein, the large degree of HLA polymorphism is an important factor to be taken into account with the epitope-based approach to developing therapeutics and diagnostics. To address this factor, epitope selection encompassing identification of peptides capable of binding at high or intermediate affinity to multiple HLA molecules is preferably utilized, most preferably these epitopes bind at high or intermediate affinity to two or more allele-specific HLA molecules. However, in some embodiments, it is preferred that all epitopes in a given composition bind to the alleles of a single HLA supertype or a single HLA molecule.

Variants of the invention preferably include those that have an IC₅₀ or binding affinity value for a class I HLA molecule(s) of 500 nM or better (i.e., the value is ≦500 nM). In certain embodiments of the invention, peptides of interest have an IC₅₀ or binding affinity value for a class I HLA molecule(s) of 200 nM or better. In certain embodiments of the invention, peptides of interest, such as A1 and A24 peptides, have an IC₅₀ or binding affinity value for a class I HLA molecule(s) of 100 nM or better. If HTL epitopes are included, they preferably are HTL epitopes that have an IC₅₀ or binding affinity value for class II HLA molecules of 1000 nM or better, (i.e., the value is ≦1,000 nM). For example, peptide binding is assessed by testing the capacity of a candidate peptide to bind to a purified HLA molecule in vitro. Peptides exhibiting high or intermediate affinity are then considered for further analysis. Selected peptides are generally tested on other members of the supertype family. In preferred embodiments, peptides that exhibit cross-reactive binding are then used in cellular screening analyses or vaccines.

The relationship between binding affinity for HLA class I molecules and immunogenicity of discrete peptide epitopes on bound antigens was determined for the first time by inventors at Epimmune. As disclosed in greater detail herein, higher HLA binding affinity is correlated with greater immunogenicity.

Greater immunogenicity can be manifested in several different ways. Immunogenicity corresponds to whether an immune response is elicited at all, and to the vigor of any particular response, as well as to the extent of a population in which a response is elicited. For example, a peptide might elicit an immune response in a diverse array of the population, yet in no instance produce a vigorous response. In accordance with these principles, close to 90% of high binding peptides have been found to elicit a response and thus be “immunogenic,” as contrasted with about 50% of the peptides that bind with intermediate affinity. (See, e.g., Schaeffer et al. PNAS (1988)) High affinity-binding class I peptides generally have an affinity of less than or equal to 100 nM. Moreover, not only did peptides with higher binding affinity have an enhanced probability of generating an immune response, the generated response tended to be more vigorous than the response seen with weaker binding peptides. As a result, less peptide is required to elicit a similar biological effect if a high affinity binding peptide is used rather than a lower affinity one. Thus, in some preferred embodiments of the invention, high affinity binding epitopes are used.

The correlation between binding affinity and immunogenicity was analyzed by the present inventors by two different experimental approaches (see, e.g., Sette, et al., J. Immunol. 153:5586-5592 (1994)). In the first approach, the immunogenicity of potential epitopes ranging in HLA binding affinity over a 10,000-fold range was analyzed in HLA-A*0201 transgenic mice. In the second approach, the antigenicity of approximately 100 different hepatitis B virus (HIV)-derived potential epitopes, all carrying A*0201 binding motifs, was assessed by using PBL from acute hepatitis patients. Pursuant to these approaches, it was determined that an affinity threshold value of approximately 500 nM (preferably 50 nM or less) determines the capacity of a peptide epitope to elicit a CTL response. These data are true for class I binding affinity measurements for naturally processed peptides and for synthesized T cell epitopes. These data also indicate the important role of determinant selection in the shaping of T cell responses (see, e.g., Schaeffer et al. Proc. Natl. Acad. Sci. USA 86:4649-4653 (1989)).

An affinity threshold associated with immunogenicity in the context of HLA class II (i.e., HLA DR) molecules has also been delineated (see, e.g., Southwood et al. J. Immunology 160:3363-3373 (1998), and U.S. Pat. No. 6,413,527, issued Jul. 2, 2002). In order to define a biologically significant threshold of HLA class II binding affinity, a database of the binding affinities of 32 DR-restricted epitopes for their restricting element (i.e., the HLA molecule that binds the epitope) was compiled. In approximately half of the cases (15 of 32 epitopes), DR restriction was associated with high binding affinities, i.e. binding affinity values of 100 nM or less. In the other half of the cases (16 of 32), DR restriction was associated with intermediate affinity (binding affinity values in the 100-1000 nM range). In only one of 32 cases was DR restriction associated with an IC₅₀ of 1000 nM or greater. Thus, 1000 nM is defined as an affinity threshold associated with immunogenicity in the context of DR molecules.

The binding affinity of peptides for HLA molecules can be determined as described in Example 1, below.

Enhancing Population Coverage of the Vaccine

The primary anchor residues of the HLA class I peptide epitope supermotifs and motifs are summarized in Tables 1-2. Allele-specific HLA molecules that are comprised by the various HLA class I supertypes are listed in Table 4. In some cases, patterns of amino acid residues are present in both a motif and a supermotif. The relationship of a particular motif and any related supermotif is indicated in the description of the individual motifs.

By inclusion of one or more, epitopes from several motifs or supermotifs in a vaccine composition, enhanced population coverage for major global ethnicities can be obtained.

Assays to Detect T-Cell Responses

Once HLA binding peptides are identified, they can be tested for the ability to elicit a T-cell response. The preparation and evaluation of motif-bearing peptides are described, e.g., in PCT publications WO 94/20127 and WO 94/03205. Briefly, peptides comprising epitopes from a particular antigen are synthesized and tested for their ability to bind to relevant HLA proteins. These assays may involve evaluation of peptide binding to purified HLA class I molecules in relation to the binding of a radioiodinated reference peptide. Alternatively, cells expressing empty class I molecules (i.e. cell surface HLA molecules that lack any bound peptide) may be evaluated for peptide binding by immunofluorescent staining and flow microfluorimetry. Other assays that may be used to evaluate peptide binding include peptide-dependent class I assembly assays and/or the inhibition of CTL recognition by peptide competition. Those peptides that bind to an HLA class I molecule, typically with an affinity of 500 nM or less, are further evaluated for their ability to serve as targets for CTLs derived from infected or immunized individuals, as well as for their capacity to induce primary in vitro or in vivo CTL responses that can give rise to CTL populations capable of reacting with selected target cells associated with pathology.

Analogous assays are used for evaluation of HLA class II binding peptides. HLA class II motif-bearing peptides that are shown to bind, typically at an affinity of 1000 nM or less, are further evaluated for the ability to stimulate HTL responses.

Conventional assays utilized to detect T cell responses include proliferation assays, lymphokine secretion assays, direct cytotoxicity assays, and limiting dilution assays. For example, antigen-presenting cells that have been incubated with a peptide can be assayed for the ability to induce CTL responses in responder cell populations. Antigen-presenting cells can be normal cells such as peripheral blood mononuclear cells or dendritic cells. Alternatively, mutant, non-human mammalian cell lines that have been transfected with a human class I MHC gene, and that are deficient in their ability to load class I molecules with internally processed peptides, are used to evaluate the capacity of the peptide to induce in vitro primary CTL responses. Peripheral blood mononuclear cells (PBMCs) can be used as the source of CTL precursors. Antigen presenting cells are incubated with peptide, after which the peptide-loaded antigen-presenting cells are then incubated with the responder cell population under optimized culture conditions. Positive CTL activation can be determined by assaying the culture for the presence of CTLs that lyse radio-labeled target cells, either specific peptide-pulsed targets or target cells that express endogenously processed antigen from which the specific peptide was derived. Alternatively, the presence of epitope-specific CTLs can be determined by IFNγ in situ ELISA.

In an embodiment of the invention, directed to diagnostics, a method has been devised which allows direct quantification of antigen-specific T cells by staining with fluorescein-labelled HLA tetrameric complexes (Altman, J. D. et al., Proc. Natl. Acad. Sci. USA 90:10330, 1993; Altman, J. D. et al., Science 274:94, 1996). Other options include staining for intracellular lymphokines, and interferon release assays or ELISPOT assays. Tetramer staining, intracellular lymphokine staining and ELISPOT assays all appear to be at least 10-fold more sensitive than more conventional assays (Lalvani, A. et al., J. Exp. Med. 186:859, 1997; Dunbar, P. R. et al., Curr. Biol. 8:413, 1998; Murali-Krishna, K. et al., Immunity 8:177, 1998). Additionally, DimerX technology can be used as a means of quantitation (see, e.g., Science 274:94-99 (1996) and Proc. Natl. Acad. Sci. 95:7568-73 (1998)).

HTL activation may also be assessed using techniques known to those in the art, such as T cell proliferation or lymphokine secretion (see, e.g. Alexander et al., Immunity 1:751-761, 1994).

Alternatively, immunization of HLA transgenic mice can be used to determine immunogenicity of peptide epitopes. Several transgenic mouse strains, e.g., mice with human A2.1, A11 (which can additionally be used to analyze HLA-A3 epitopes), and B7 alleles have been characterized. Other transgenic mice strains (e.g. transgenic mice for HLA-A1 and A24) are being developed. Moreover, HLA-DR1 and HLA-DR3 mouse models have been developed. In accordance with principles in the art, additional transgenic mouse models with other HLA alleles are generated as necessary.

Such mice can be immunized with peptides emulsified in Incomplete Freund's Adjuvant; thereafter any resulting T cells can be tested for their capacity to recognize target cells that have been peptide-pulsed or transfected with genes encoding the peptide of interest. CTL responses can be analyzed using cytotoxicity assays described above. Similarly, HTL responses can be analyzed using, e.g., T cell proliferation or lymphokine secretion assays.

Minigenes

A number of different approaches are available which allow simultaneous delivery of multiple epitopes. Nucleic acids encoding multiple epitopes are a useful embodiment of the invention; discrete peptide epitopes or polyepitopic peptides can be encoded. The epitopes to be included in a minigene are preferably selected according to the guidelines set forth in the previous section. Examples of amino acid sequences that can be included in a minigene include: HLA class I epitopes, HLA class II epitopes, a ubiquitination signal sequence, and/or a targeting sequence such as an endoplasmic reticulum (ER) signal sequence to facilitate movement of the resulting peptide into the endoplasmic reticulum. Examples of minigene constructs are shown in Tables 23-28.

The use of multi-epitope minigenes is also described in, e.g. co-pending applications U.S. Ser. No. 09/311,784, 09/894,018, 60/419,973, 60/415,463; Ishioka et al., J. Immunol. 162:3915-3925, 1999; An, L. and Whitton, J. L., J. Virol. 71:2292, 1997; Thomson, S. A. et al., J. Immunol. 157:822, 1996; Whitton, J. L. et al., J. Virol 67:348, 1993; Hanke, R. et al., Vaccine 16:426, 1998. For example, a multi-epitope DNA plasmid encoding nine dominant HLA-A*0201- and A11-restricted CTL epitopes derived from the polymerase, envelope, and core proteins of HIV and human immunodeficiency virus (HIV), a PADRE® universal helper T cell (HTL) epitope, and an endoplasmic reticulum-translocating signal sequence has been engineered. Immunization of HLA transgenic mice with this plasmid construct resulted in strong CTL induction responses against the nine CTL epitopes tested. This CTL response was similar to that observed with a lipopeptide of known immunogenicity in humans, and significantly greater than immunization using peptides in oil-based adjuvants. Moreover, the immunogenicity of DNA-encoded epitopes in vitro was also correlated with the in vitro responses of specific CTL lines against target cells transfected with the DNA plasmid. These data show that the minigene served: 1.) to generate a CTL response and 2.) to generate CTLs that recognized cells expressing the encoded epitopes. A similar approach can be used to develop minigenes encoding epitopes of an infectious agent.

For example, to create a DNA sequence encoding the selected epitopes (minigene) for expression in human cells, the amino acid sequences of the epitopes may be reverse translated. A human codon usage table can be used to guide the codon choice for each amino acid. These epitope-encoding DNA sequences may be directly adjoined, so that when translated, a continuous peptide sequence is created. However, to optimize expression and/or immunogenicity, additional elements can be incorporated into the minigene design such as spacer amino acid residues between epitopes. HLA presentation of CTL and HTL epitopes may be improved by including synthetic (e.g. poly-alanine) or naturally-occurring flanking sequences adjacent to the CTL or HTL epitopes; these larger peptides comprising the epitope(s) are within the scope of the invention. In one embodiment, spacer amino acid residues between one or more CTL and/or HTL epitopes are designed so as to minimize junctional epitopes that may result from the juxtaposition of 2 CTL and/or HTL epitopes.

The minigene sequence may be converted to DNA by assembling oligonucleotides that encode the plus and minus strands of the minigene. Overlapping oligonucleotides (30-100 bases long) may be synthesized, phosphorylated, purified and annealed under appropriate conditions using well known techniques. The ends of the oligonucleotides can be joined, for example, using T4 DNA ligase. This synthetic minigene, encoding the epitope peptide, can then be cloned into a desired expression vector.

Standard regulatory sequences well known to those of skill in the art are preferably included in the vector to ensure expression in the target cells. Several vector elements are desirable: a promoter with a downstream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) CMV-IE promoter. See, e.g., U.S. Pat. Nos. 5,580,859 and 5,589,466 for other suitable promoter sequences.

Optimized peptide expression and immunogenicity can be achieved by certain modifications to a minigene construct. For example, in some cases introns facilitate efficient gene expression, thus one or more synthetic or naturally-occurring introns can be incorporated into the transcribed region of the minigene. The inclusion of mRNA stabilization sequences and sequences for replication in mammalian cells may also be considered for increasing minigene expression.

Once an expression vector is selected, the minigene is cloned into the polylinker region downstream of the promoter. This plasmid is transformed into an appropriate bacterial strain, and DNA is prepared using standard techniques. The orientation and DNA sequence of the minigene, as well as all other elements included in the vector, are confirmed using restriction mapping, PCR and/or DNA sequence analysis. Bacterial cells harboring the correct plasmid can be stored as cell banks.

In addition, immunostimulatory sequences (ISSs or CpGs) appear to play a role in the immunogenicity of DNA vaccines. These sequences may be included in the vector, outside the minigene coding sequence to enhance immunogenicity.

In some embodiments, a bi-cistronic expression vector which allows production of both the minigene-encoded epitopes and a second protein (e.g. one that modulates immunogenicity) can be used. Examples of proteins or polypeptides that, if co-expressed with epitopes, can enhance an immune response include cytokines (e.g., IL-2, IL-12, GM-CSF), cytokine-inducing molecules (e.g., LeIF), costimulatory molecules, or pan-DR binding proteins (PADRE®, Epimmune, San Diego, Calif.). Helper T cell (HTL) epitopes such as PADRE® molecules can be joined to intracellular targeting signals and expressed separately from expressed CTL epitopes. This can be done in order to direct HTL epitopes to a cell compartment different than that of the CTL epitopes, one that provides for more efficient entry of HTL epitopes into the HLA class II pathway, thereby improving HTL induction. In contrast to HTL or CTL induction, specifically decreasing the immune response by co-expression of immunosuppressive molecules (e.g. TGF-β) may be beneficial in certain diseases.

Therapeutic quantities of plasmid DNA can be produced for example, by fermentation in E. coli, followed by purification. Aliquots from the working cell bank are used to inoculate growth medium, and are grown to saturation in shaker flasks or a bioreactor according to well known techniques. Plasmid DNA is purified using standard bioseparation technologies such as solid phase anion-exchange resins available, e.g., from QIAGEN, Inc. (Valencia, Calif.). If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is currently being used for intramuscular (IM) administration in clinical trials. To maximize the immunotherapeutic effects of minigene vaccines, alternative methods of formulating purified plasmid DNA may be used. A variety of such methods have been described, and new techniques may become available. Cationic lipids, glycolipids, and fusogenic liposomes can also be used in the formulation (see, e.g., WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In addition, peptides and compounds referred to collectively as protective, interactive, non-condensing compounds (PINC) can also be complexed to purified plasmid DNA to influence variables such as stability, intramuscular dispersion, or trafficking to specific organs or cell types.

Known methods in the art can be used to enhance delivery and uptake of a polynucleotide in vivo. For example, the polynucleotide can be complexed to polyvinylpyrrolidone (PVP), to prolong the localized bioavailability of the polynucleotide, thereby enhancing uptake of the polynucleotide by the organisum (see e.g. U.S. Pat. No. 6,040,295; EP 0 465 529; WO 98/17814). PVP is a polyamide that is known to form complexes with a wide variety of substances, and is chemically and physiologically inert.

Target cell sensitization can be used as a functional assay of the expression and HLA class I presentation of minigene-encoded epitopes. For example, the plasmid DNA is introduced into a mammalian cell line that is a suitable target for standard CTL chromium release assays. The transfection method used will be dependent on the final formulation, electroporation can be used for “naked” DNA, whereas cationic lipids or DNA:PVP compositions allow direct in vitro transfection. A plasmid expressing green fluorescent protein (GFP) can be co-transfected to allow enrichment of transfected cells using fluorescence activated cell sorting (FACS). The transfected cells are then chromium-51 (⁵¹Cr) labeled and used as targets for epitope-specific CTLs. Cytolysis of the target cells, detected by ⁵¹Cr release, indicates both the production and HLA presentation of, minigene-encoded CTL epitopes. Expression of HTL epitopes may be evaluated in an analogous manner using assays to assess HTL activity.

In vivo immunogenicity is a second approach for functional testing of minigene DNA formulations. Transgenic mice expressing appropriate human HLA proteins are immunized with the DNA product. The dose and route of administration are formulation dependent (e.g., IM for DNA in PBS, intraperitoneal (IP) for lipid-complexed DNA). Eleven to twenty-one days after immunization, splenocytes are harvested and restimulated for one week in the presence of peptides encoding each epitope being tested. Thereafter, for CTLs, standard assays are conducted to determine if there is cytolysis of peptide-loaded, ⁵¹Cr-labeled target cells. Once again, lysis of target cells that were exposed to epitopes corresponding to those in the minigene, demonstrates DNA vaccine function and induction of CTLs. Immunogenicity of HTL epitopes is evaluated in transgenic mice in an analogous manner.

Alternatively, the nucleic acids can be administered using ballistic delivery as described, for instance, in U.S. Pat. No. 5,204,253. Using this technique, particles comprised solely of DNA are administered. In a further alternative embodiment for ballistic delivery, DNA can be adhered to particles, such as gold particles.

Vaccine Compositions

Vaccines that contain an immunologically effective amount of one or more peptides or polynucleotides of the invention are a further embodiment of the invention. The peptides can be delivered by various means or formulations, all collectively referred to as “vaccine” compositions. Such vaccine compositions, and/or modes of administration, can include, for example, naked DNA, DNA formulated with PVP, DNA in cationic lipid formulations; lipopeptides (e.g., Vitiello, A. et al., J. Clin. Invest. 95:341, 1995), DNA or peptides, encapsulated e.g., in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al.., Vaccine 13:675-681, 1995); peptide compositions contained in immune stimulating complexes (ISCOMS) (see, e.g., Takahashi et al., Nature 344:873-875, 1990; Hu et al., Clin Exp Immunol. 113:235-243, 1998); multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996); viral, bacterial, or, fungal delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990); particles of viral or synthetic origin (e.g., Kofler, N. et al, J. Immunol. Methods. 192:25, 1996; Eldridge, J. H. et al., Sem. Hematol. 30:16, 1993; Falo, L. D., Jr. et al., Nature Med. 7:649, 1995); adjuvants (e.g., incomplete freund's advjuvant) (Warren, H. S., Vogel, F. R., and Chedid, L. A. Annu. Rev. Immunol. 4:369, 1986; Gupta, R. K. et al., Vaccine 11:293, 1993); liposomes (Reddy, R. et al., J. Immunol. 148:1585, 1992; Rock, K. L., Immunol. Today 17:131, 1996); or, particle-absorbed DNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993), etc. Toxin-targeted delivery technologies, also known as receptor mediated targeting, such as those of Avant Immunotherapeutics, Inc. (Needham, Mass.) or attached to a stress protein, e.g., HSP 96 (Stressgen Biotechnologies Corp., Victoria, BC, Canada) can also be used.

Vaccines of the invention comprise nucleic acid mediated modalities. DNA or RNA encoding one or more of the peptides of the invention can be administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; and, WO 98/04720. Examples of DNA-based delivery technologies include “naked DNA”, facilitated (bupivicaine, polymers (e.g., PVP), peptide-mediated) delivery, cationic lipid complexes, and particle-mediated (“gene gun”) or pressure-mediated delivery (see, e.g., U.S. Pat. No. 5,922,687). Accordingly, peptide vaccines of the invention can be expressed by viral or bacterial vectors. Examples of expression vectors include attenuated viral hosts, such as vaccinia or fowlpox. For example, vaccinia virus is used as a vector to express nucleotide sequences that encode the peptides of the invention (e.g., MVA). Upon introduction into an acutely or chronically infected host or into a non-infected host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the peptides of the invention, e.g. adeno and adeno-associated virus vectors, alpha virus vectors, retroviral vectors, Salmonella typhi vectors, detoxified anthrax toxin vectors, and the like, are apparent to those skilled in the art from the description herein.

Furthermore, vaccines in accordance with the invention can comprise one or more peptides of the invention. Accordingly, a peptide can be present in a vaccine individually; alternatively, the peptide can exist as a homopolymer comprising multiple copies of the same peptide, or as a heteropolymer of various peptides. Polymers have the advantage of increased probability for immunological reaction and, where different peptide epitopes are used to make up the polymer, the ability to induce antibodies and/or T cells that react with different antigenic determinants of the antigen targeted for an immune response. The composition may be a naturally occurring region of an antigen or can be prepared, e.g., recombinantly or by chemical synthesis.

Carriers that can be used with vaccines of the invention are well known in the art, and include, e.g., thyroglobulin, albumins such as human serum albumin, tetanus toxoid, polyamino acids such as poly L-lysine, poly L-glutamic acid, influenza virus proteins, hepatitis B virus core protein, and the like. The vaccines can contain a physiologically tolerable diluent such as water, or a saline solution, preferably phosphate buffered saline. Generally, the vaccines also include an adjuvant. Adjuvants such as incomplete Freund's adjuvant, aluminum phosphate, aluminum hydroxide, or alum are examples of materials well known in the art. Additionally, as disclosed herein, CTL responses can be primed by conjugating peptides of the invention to lipids, such as tripalmitoyl-S-glyceryl-cysteinyl-seryl-serine (P₃CSS).

Upon immunization with a peptide composition in accordance with the invention, via injection (e.g., SC, ID, IM), aerosol, oral, transdermal, transmucosal, intrapleural, intrathecal, or other suitable routes, the immune system of the host responds to the vaccine by producing antibodies, CTLs and/or HTLs specific for the desired antigen. Consequently, the host becomes at least partially immune to subsequent exposure to the infectious agent, and thereby derives a prophylactic or therapeutic benefit.

In certain embodiments, components that induce T cell responses are combined with components that induce antibody responses to the target antigen of interest. A preferred embodiment of such a composition comprises class I and class II epitopes in accordance with the invention. Alternatively, a composition comprises a class I and/or class II epitope in accordance with the invention, along with a PADRE® molecule (Epimmune, San Diego, Calif.).

Vaccines of the invention can comprise antigen presenting cells, such as dendritic cells, as a vehicle to present peptides of the invention. For example, dendritic cells are transfected, e.g., with a minigene construct in accordance with the invention, in order to elicit immune responses. Minigenes are discussed in greater detail in a following section. Vaccine compositions can be created in vitro, following dendritic cell mobilization and harvesting, whereby loading of dendritic cells occurs in vitro.

The vaccine compositions of the invention may also be used in combination with antiviral drugs such as interferon-α, or immune adjuvants such as IL-12, GM-CSF, etc.

Preferably, the following principles are utilized when selecting epitope(s) and/or analogs for inclusion in a vaccine, either peptide-based or nucleic acid-based formulations. Exemplary variants that may be utilized in a vaccine to treat or prevent infectious agent-mediated disease are set out in Tables 6-9 and FIGS. 1A-4. Each of the following principles can be balanced in order to make the selection. When multiple epitopes are to be used in a vaccine, the epitopes may be, but need not be, contiguous in sequence in the native antigen from which the epitopes are derived. Such multiple epitotes can refer to the order of epitopes within a peptide, or to the selection of epitopes that come from the same reagion, for use in either individual peptides or in a multi-epitopic peptide.

1.) Variants are selected which, upon administration, mimic immune responses that have been observed to be correlated with prevention or clearance of infectious disease. For HLA Class I, this generally includes 3-7 variants from at least one infectious agent or antigen thereof.

2.) Variants are selected that have the requisite binding affinity established to be correlated with immunogenicity: for HLA Class I an IC₅₀ of 500 nM or less, or for Class II an IC₅₀ of 1000 nM or less. For HLA Class I it is presently preferred to select a peptide having an IC₅₀ of 200 nM or less, as this is believed to better correlate not only to induction of an immune response, but to in vitro tumor cell killing as well. For HLA A1 and A24, it is especially preferred to select a peptide having an IC₅₀ of 100 nM or less.

3.) Supermotif bearing-variants, or a sufficient array of allele-specific motif-bearing variants, are selected to give broad population coverage. In general, it is preferable to have at least 80% population coverage. A Monte Carlo analysis, a statistical evaluation known in the art, can be employed to assess the breadth of population coverage.

4.) Of particular relevance are “nested epitopes.” Nested epitopes occur where at least two epitopes overlap in a given peptide sequence. For example, a nested epitope can be a fragment of an antigen from a region that contains multiple epitopes that are overleapping, or one epitope that is completely encompassed by another, e.g., A2 peptides MAGE3.159 and MAGE3.160 are nested epitopes. A peptide comprising “transcendent nested epitopes” is a peptide that has both HLA class I and HLA class II epitopes in it. When providing nested epitopes, it is preferable to provide a sequence that has the greatest number of epitopes per provided sequence. Preferably, one avoids providing a peptide that is any longer than the amino terminus of the amino terminal epitope and the carboxyl terminus of the carboxyl terminal epitope in the peptide. When providing a sequence comprising nested epitopes, it is important to evaluate the sequence in order to insure that it does not have pathological or other deleterious biological properties; this is particularly relevant for vaccines directed to infectious organisms.

5.) If a protein with multiple epitopes or a polynucleotide (e.g., minigene) is created, an objective is to generate the smallest peptide that encompasses the epitopes of interest. This principle is similar, if not the same as that employed when selecting a peptide comprising nested epitopes. However, with an artificial peptide comprising multipe epitopes, the size minimization objective is balanced against the need to integrate any spacer sequences between epitopes in the polyepitopic protein. Spacer amino acid residues can be introduced to avoid junctional epitopes (an epitope recognized by the immune system, not present in the target antigen, and only created by the man-made juxtaposition of epitopes), or to facilitate cleavage between epitopes and thereby enhance epitope presentation. Junctional epitopes are generally to be avoided because the recipient may generate an immune response to that non-native epitope. Of particular concern is a junctional epitope that is a “dominant epitope.” A dominant epitope may lead to such a zealous response that immune responses to other epitopes are diminished or suppressed.

The principles are the same, except junctional epitopes applies to the sequences surrounding the epitope. One must also take care with other sequences in construct to avoid immune response.

T Cell Priming Materials

In some embodiments it may be desirable to include in the pharmaceutical compositions of the invention at least one component which primes cytotoxic T lymphocytes. Lipids have been identified as agents capable of facilitating the priming in vitro CTL response against viral antigens. For example, palmitic acid residues can be attached to the ε- and α-amino groups of a lysine residue and then linked to an immunogenic peptide. One or more linking moieties can be used such as Gly, Gly-Gly-, Ser, Ser-Ser, or the like. The lipidated peptide can then be administered directly in a micelle or particle, incorporated into a liposome, or emulsified in an adjuvant, e.g., incomplete Freund's adjuvant. A preferred immunogenic composition comprises palmitic acid attached to ε- and α-amino groups of Lys via a linking moiety, e.g., Ser-Ser, added to the amino terminus of an immunogenic peptide.

In another embodiment of lipid-facilitated priming of CTL responses, E. coli lipoproteins, such as tripalmitoyl-S-glyceryl-cysteinyl-seryl-serine (P₃CSS) can be used to prime CTL when covalently attached to an appropriate peptide. (See, e.g., Deres, et al., Nature 342:561, 1989). Thus, peptides of the invention can be coupled to P₃CSS, and the lipopeptide administered to an individual to specifically prime a CTL response to the target antigen. Moreover, because the induction of neutralizing antibodies can also be primed with P₃CSS-conjugated epitopes, two such compositions can be combined to elicit both humoral and cell-mediated responses.

Dendritic Cells Pulsed with CTL and/or HTL Peptides

An embodiment of a vaccine composition in accordance with the invention comprises ex vivo administration of a cocktail of epitope-bearing peptides to PBMC, or isolated DC therefrom, from the patient's blood. A pharmaceutical to facilitate harvesting of DC can be used, such as Progenipoietin™ (Monsanto, St. Louis, Mo.) or GM-CSF/IL-4. After pulsing the DC with peptides and prior to reinfusion into patients, the DC are washed to remove unbound peptides. In this embodiment, a vaccine comprises peptide-pulsed DCs which present the pulsed peptide epitopes in HLA molecules on their surfaces.

The DC can be pulsed ex vivo with a cocktail of peptides, some of which stimulate CTL responses to one or more antigens of interest, e.g., antigens from infectious agents such as HIV env, HIV pol, HIV gag, HIV vpu, HIV and/or the antigens in Tables 11-22, or otherwise described herein or know in the art. Optionally, a helper T cell (HTL) peptide such as PADRE®, can be included to facilitate the CTL response. Thus, a vaccine in accordance with the invention comprising epitopes from an infectious agent is used to treat or prevent disease mediated by these agents in patients. A vaccine can be used prior to, during, or following other therapies including, for example, antibiotic therepy, anti-viral therapy (e.g., highly active antiretroviral therapy (HAART) in the case of HIV-AIDS), antibody therapy, cancer therapy, and adjunct thereapy, whereupon the vaccine provides descreased morbidity, increased disease free survival and overall survival in recipients.

Diagnostic and Prognostic Uses

In one embodiment of the invention, HLA class I and class II binding peptides can be used as reagents to evaluate an immune response. Preferably, the following principles are utilized when selecting a variant(s) for diagnostic, prognostic and similar uses. Potential principles include having the binding affinities described earlier, and/or matching the HLA-motif/supermotif of a peptide with the HLA-type of a patient.

The evaluated immune response can be induced by any immunogen. For example, the immunogen may result in the production of antigen-specific CTLs or HTLs that recognize the peptide epitope(s) employed as the reagent. Thus, a peptide of the invention may or may not be used as the immunogen. Assay systems that can be used for such analyses include tetramer-based protocols (e.g., DimerX technology (see, e.g., Science 274:94-99 (1996) and Proc. Natl. Acad. Sci. 95:7568-73 (1998)), staining for intracellular lymphokines, interferon release assays, or ELISPOT assays.

For example, following exposure to a putative immunogen, a peptide of the invention can be used in a tetramer staining assay to assess peripheral blood mononuclear cells for the presence of any antigen-specific CTLs. The HLA-tetrameric complex is used to directly visualize antigen-specific CTLs and thereby determine the frequency of such antigen-specific CTLs in a sample of peripheral blood mononuclear cells (see, e.g., Ogg et al., Science 279:2103-2106, 1998; and Altman et al., Science 174:94-96, 1996).

A tetramer reagent comprising a peptide of the invention is generated as follows: A peptide that binds to an HLA molecule is refolded in the presence of the corresponding. HLA heavy chain and β₂-microglobulin to generate a trimolecular complex. The complex is biotinylated at the carboxyl terminal end of the HLA heavy chain, at a site that was previously engineered into the protein. Tetramer formation is then induced by adding streptavidin. When fluorescently labeled streptavidin is used, the tetrameric complex is used to stain antigen-specific cells. The labeled cells are then readily identified, e.g. by flow cytometry. Such procedures are used for diagnostic or prognostic purposes; the cells identified by the procedure can be used for therapeutic purposes.

Peptides of the invention are also used as reagents to evaluate immune recall responses. (see, e.g., Bertoni et al., J. Clin. Invest. 100:503-513, 1997 and Penna et al., J. Exp. Med. 174:1565-1570, 1991.) For example, a PBMC sample from an individual expressing a disease-associated antigen (e.g. an antigen from an infectious agent) can be analyzed for the presence of antigen-specific CTLs or HTLs using specific peptides. A blood sample containing mononuclear cells may be evaluated by cultivating the PBMCs and stimulating the cells with a peptide of the invention. After an appropriate cultivation period, the expanded cell population may be analyzed, for example, for CTL or for HTL activity.

Thus, the peptides can be used to evaluate the efficacy of a vaccine. PBMCs obtained from a patient vaccinated with an immunogen may be analyzed by methods such as those described herein. The patient is HLA typed, and peptide epitopes that are bound by the HLA molecule(s) present in that patient are selected for analysis. The immunogenicity of the vaccine is indicated by the presence of CTLs and/or HTLs directed to epitopes present in the vaccine.

The peptides of the invention may also be used to make antibodies, using techniques well known in the art (see, e.g. CURRENT PROTOCOLS IN IMMUNOLOGY, Wiley/Greene, NY; and Antibodies A Laboratory Manual Harlow, Harlow and Lane, Cold Spring Harbor Laboratory Press, 1989). Such antibodies are useful as reagents to determine the presence of disease-associated antigens. Antibodies in this category include those that recognize a peptide when bound by an HLA molecule, i.e., antibodies that bind to a peptide-MHC complex.

Administration for Therapeutic or Prophylactic Purposes

The peptides and polynucleotides of the present invention, including cells and compositions comprising them, are useful for administration to mammals, particularly humans, to treat and/or prevent infection by an infectious agent such as HIV, HBV, HCV, HPV, Plasmodium falciparum and other agents described herein or known in the art. Vaccine compositions containing the peptides of the invention are administered to a patient infected with a particular infectious agent or to an individual susceptible to, or otherwise at risk for, infection with such an agent to elicit an immune response against antigens of that agent and thus enhance the patient's own immune response capabilities. Where susceptible individuals are identified prior to infection, the composition can be targeted to them, thus minimizing the need for administration to a larger population.

In therapeutic applications, peptide and/or nucleic acid compositions are administered to a patient in an amount sufficient to elicit an effective immune response to the infectious agent antigen and to thereby cure, arrest or slow symptoms and/or complications. An amount adequate to accomplish this is defined as “therapeutically effective dose.” Amounts effective for this use will depend on, e.g., the particular composition administered, the manner of administration, the stage and severity of the disease being treated, the weight and general state of health of the patient, and the judgment of the prescribing physician.

The vaccine compositions of the invention can be used purely as prophylactic agents. Generally the dosage for an initial prophylactic immunization generally occurs in a unit dosage range where the lower value is about 1, 5, 50, 500, or 1000 μg of peptide and the higher value is about 10,000; 20,000; 30,000; or 50,000 μg of peptide. Dosage values for a human typically range from about 500 μg to about 50,000 μg of peptide per 70 kilogram patient. This is followed by boosting dosages of between about 1.0 μg to about 50,000 μg of peptide, administered at defined intervals from about four weeks to six months after the initial administration of vaccine. The immunogenicity of the vaccine may be assessed by measuring the specific activity of CTL and HTL obtained from a sample of the patient's blood.

As noted above, peptides comprising CTL and/or HTL epitopes of the invention induce immune responses when presented by HLA molecules and contacted with a CTL or HTL specific for an epitope comprised by the peptide. The manner in which the peptide is contacted with the CTL or HTL is not critical to the invention. For instance, the peptide can be contacted with the CTL or HTL either in vitro or in vivo. If the contacting occurs in vivo, peptide can be administered directly, or in other forms/vehicles, e.g. DNA vectors encoding one or more peptides, viral vectors encoding the peptide(s), liposomes, antigen presenting cells such as dendritic cells, and the like.

Accordingly, for pharmaceutical compositions of the invention in the form of peptides or polypeptides, the peptides or polypeptides can be administered directly. Alternatively, the peptide/polypeptides can be administered indirectly presented on APCs, or as DNA encoding them. Furthermore, the peptides or DNA encoding them can be administered individually or as fusions of one or more peptide sequences.

For therapeutic use, administration should generally begin at the first diagnosis of infectious agent-related disease. This is followed by boosting doses at least until symptoms are substantially abated and for a period thereafter. In chronic disease states, loading doses followed by boosting doses may be required.

The dosage for an initial therapeutic immunization generally occurs in a unit dosage range where the lower value is about 1, 5, 50, 500, or 1,000 μg of peptide and the higher value is about 10,000; 20,000; 30,000; or 50,000 μg of peptide. Dosage values for a human typically range from about 500 μg to about 50,000 μg of peptide per 70 kilogram patient. Boosting dosages of between about 1.0 μg to about 50,000 μg of peptide, administered pursuant to a boosting regimen over weeks to months, can be administered depending upon the patient's response and condition. Patient response can be determined by measuring the specific activity of CTL and HTL obtained from the patient's blood.

In certain embodiments, peptides and compositions of the present invention are used in serious disease states. In such cases, as a result of the minimal amounts of extraneous substances and the relative nontoxic nature of the peptides, it is possible and may be desirable to administer substantial excesses of these peptide compositions relative to these stated dosage amounts.

For treatment of chronic disease, a representative dose is in the range disclosed above, namely where the lower value is about 1, 5, 50, 500, or 1,000 μg of peptide and the higher value is about −10,000; 20,000; 30,000; or 50,000 μg of peptide, preferably from about 500 μg to about 50,000 μg of peptide per 70 kilogram patient. Initial doses followed by boosting doses at established intervals, e.g., from four weeks to six months, may be required, possibly for a prolonged period of time to effectively immunize an individual. In the case of chronic disease, administration should continue until at least clinical symptoms or laboratory tests indicate that the disease has been eliminated or substantially abated, and for a follow-up period thereafter. The dosages, routes of administration, and dose schedules are adjusted in accordance with methodologies known in the art.

The pharmaceutical compositions for therapeutic treatment are intended for parenteral, topical, oral, intrathecal, or local administration. Preferably, the pharmaceutical compositions are administered parentally, e.g., intravenously, subcutaneously, intradermally, or intramuscularly.

Thus, in a preferred embodiment the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances or pharmaceutical excipients as may be required to approximate physiological conditions, such as pH-adjusting and buffering agents, tonicity adjusting agents, wetting agents, preservatives, and the like, for example, sodium acetate, sodium lactate, sodium chloride, potassium chloride, calcium chloride, sorbitan monolaurate, triethanolamine oleate, etc.

The concentration of peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

A human unit dose form of the peptide composition is typically included in a pharmaceutical composition that also comprises a human unit dose of an acceptable carrier, preferably an aqueous carrier, and is administered in a volume of fluid that is known by those of skill in the art to be used for administration of such compositions to humans (see, e.g., Reminigton's Pharmaceutical Sciences, 17^{th} Edition, A. Gennaro, Editor, Mack Publishing Co., Easton, Pa., 1985).

The peptides of the invention can also be administered via liposomes, which serve to target the peptides to a particular tissue, such as lymphoid tissue, or to target selectively to infected cells, as well as to increase the half-life of the peptide composition. Liposomes include emulsions, foams, micelles, insoluble monolayers, liquid crystals, phospholipid dispersions, lamellar layers and the like. In these preparations, the peptide to be delivered is incorporated as part of a liposome, alone or in conjunction with a molecule which binds to a receptor prevalent among lymphoid cells (such as monoclonal antibodies which bind to the CD45 antigen) or with other therapeutic or immunogenic compositions. Thus, liposomes either filled or decorated with a desired peptide of the invention can be directed to the site of lymphoid cells, where the liposomes then deliver the peptide compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), and U.S. Pat. Nos. 4,235,871, 4,501,728, 4,837,028, and 5,019,369.

For targeting compositions of the invention to cells of the immune system, a ligand can be incorporated into the liposome, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, often at a concentration of 25%-75%.

For aerosol administration, the immunogenic peptides are preferably supplied in finely divided form, along with a surfactant and propellant. Typical percentages of peptides are 0.01%-20% by weight, often 1%-10%. The surfactant must, of course, be pharmaceutically acceptable, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant, although an atomizer may be used in which no propellant is necessary and other percentages are adjusted accordingly. A carrier can also be included, e.g., lecithin for intranasal delivery.

Antigenic peptides of the invention have been used to elicit a CTL and/or HTL response ex vivo, as well. The resulting CTLs or HTLs can be used to treat chronic infections, or tumors in patients that do not respond to other conventional forms of therapy, or who do not respond to a therapeutic peptide or nucleic acid vaccine in accordance with the invention. Ex vivo CTL or HTL responses to a particular antigen (infectious or tumor-associated) are induced by incubating in tissue culture the patient's, or genetically compatible, CTL or HTL precursor cells together with a source of antigen-presenting cells (APC), such as dendritic cells, and the appropriate immunogenic peptide. After an appropriate incubation time (typically about 7-28 days), in which the precursor cells are activated and expanded into effector cells, the cells are infused back into the patient, where they will destroy (CTL) or facilitate destruction (HTL) of their specific target cell (an infected cell or a tumor cell).

Kits

The peptide and nucleic acid compositions of this invention can be provided in kit form together with instructions for vaccine administration. Typically the kit would include desired composition(s) of the invention in a container, preferably in unit dosage form and instructions for administration. For example, a kit would include an APC, such as a dendritic cell, previously exposed to and now presenting peptides of the invention in a container, preferably in unit dosage form together with instructions for administration. An alternative kit would include a minigene construct with desired nucleic acids of the invention in a container, preferably in unit dosage form together with instructions for administration. Lymphokines such as IL-2 or IL-12 may also be included in the kit. Other kit components that may also be desirable include, for example, a sterile syringe, booster dosages, and other desired excipients.

The invention will be described in greater detail by way of specific examples. The following examples are offered for illustrative purposes, and are not intended to limit the invention in any manner. Those of skill in the art will readily recognize a variety of non-critical parameters that can be changed or modified to yield alternative embodiments in accordance with the invention.

### EXAMPLES

### Example 1

### HLA Class I and Class II Binding Assays

The following example of peptide binding to HLA molecules demonstrates quantification of binding affinities of HLA class I and class II peptides. Binding assays can be performed with peptides that are either motif-bearing or not motif-bearing.

Cell lysates were prepared and HLA molecules purified in accordance with disclosed protocols (Sidney et al., Current Protocols in Immunology 18.3.1 (1998); Sidney, et al., J. Immunol. 154:247 (1995); Sette, et al., Mol. Immunol. 31:813 (1994)). The cell lines used as sources of HLA molecules and the antibodies used for the extraction of the HLA molecules from the cell lysates are also described in these publications and are well known in the art.

Epstein-Barr virus (EBV)-transformed homozygous cell lines, fibroblasts, CIR, or 721.221-transfectants were used as sources of HLA class I molecules. These cells were cultured in RPMI 1640 medium supplemented with 2 mM L-glutamine (GIBCO, Grand Island, N.Y.), 50 μM 2-ME, 100 μg/ml of streptomycin, 100 U/ml of penicillin (Irvine Scientific) and 10% heat-inactivated FCS (Irvine Scientific, Santa Ana, Calif.).

Cell lysates were prepared as follows. Briefly, cells were lysed at a concentration of 10⁸ cells/ml in 50 mM Tris-HCl, pH 8.5, containing 1% Nonidet P-40 (Fluka Biochemika, Buchs, Switzerland), 150 mM NaCl, 5 mM EDTA, and 2 mM PMSF. Lysates were cleared of debris and nuclei by centrifugation at 15,000×g for 30 min.

HLA molecules were purified from lysates by affinity chromatography. Lysates were passed twice through two pre-columns of inactivated Sepharose CL4-B and protein A-Sepharose. Next, the lysate was passed over a column of Sepharose CL-4B beads coupled to an appropriate antibody. The anti-HLA column was then washed with 10-column volumes of 10 mM Tris-HCL, pH 8.0, in 1% NP-40, PBS, 2-column volumes of PBS, and 2-column volumes of PBS containing 0.4% n-octylglucoside. Finally, MHC molecules were eluted with 50 mM diethylamine in 0.15M NaCl containing 0.4% n-octylglucoside, pH 11.5. A 1/25 volume of 2.0M Tris, pH 6.8, was added to the eluate to reduce the pH to ˜8.0. Eluates were then concentrated by centrifugation in Centriprep 30 concentrators at 2000 rpm (Amicon, Beverly, Mass.). Protein content was evaluated by a BCA protein assay (Pierce Chemical Co., Rockford, Ill.) and confirmed by SDS-PAGE.

A detailed description of the protocol utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) were incubated with various unlabeled peptide inhibitors and 1-10 nM ¹²⁵I-radiolabeled probe peptides for 48 h in PBS containing 0.05% Nonidet P-40 (NP40) (or 20% w/v digitonin for H-2 IA assays) in the presence of a protease inhibitor cocktail. The final concentrations of protease inhibitors (each from CalBioChem, La Jolla, Calif.) were 1 mM PMSF, 1.3 nM 1.10 phenanthroline, 73 μM pepstatin A, 8 mM EDTA, 6 mM N-ethylmaleimide (for Class II assays), and 200 μM N alpha-p-tosyl-L-lysine chloromethyl ketone (TLCK). All assays were performed at pH 7.0 with the exception of DRB1*0301, which was performed at pH 4.5, and DRB1*1601 (DR2w21β₁) and DRB4*0101 (DRw53), which were performed at pH 5.0. pH was adjusted as described elsewhere (see Sidney et al., in Current Protocols in Immunology, Margulies, Ed., John Wiley & Sons, New York, Section 18.3, 1998).

Following incubation, MHC-peptide complexes were separated from free peptide by gel filtration on 7.8 mm×15 cm TSK200 columns (TosoHaas 16215, Montgomeryville, Pa.), eluted at 1.2 mls/min with PBS pH 6.5 containing 0.5% NP40 and 0.1% NaN₃. Because the large size of the radiolabeled peptide used for the DRB1*1501 (DR2w2β₁) assay makes separation of bound from unbound peaks more difficult under these conditions, all DRB1*1501 (DR2w2β₁) assays were performed using a 7.8 mm×30 cm TSK2000 column eluted at 0.6 mls/min. The eluate from the TSK columns was passed through a Beckman 170 radioisotope detector, and radioactivity was plotted and integrated using a Hewlett-Packard 3396A integrator, and the fraction of peptide bound was determined.

Radiolabeled peptides were iodinated using the chloramine-T method. Representative radiolabeled probe peptides utilized in each assay, and its assay specific IC₅₀ nM, are known in the art. Typically, in preliminary experiments, each MHC preparation was titered in the presence of fixed amounts of radiolabeled peptides to determine the concentration of HLA molecules necessary to bind 10-20% of the total radioactivity. All subsequent inhibition and direct binding assays were performed using these HLA concentrations.

Since under these conditions [label]<[HLA] and IC₅₀≧[HLA], the measured IC₅₀ values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding figure is calculated for each peptide by dividing the IC₅₀ of a positive control for inhibition by the IC₅₀ for each tested peptide (typically unlabeled versions of the radiolabeled probe peptide). For inter-experiment comparisons, relative binding values are compiled. These values can subsequently be converted back into IC₅₀ nM values by dividing the IC₅₀ nM of the positive controls for inhibition by the relative binding of the peptide of interest. This method of data compilation has proven to be the most accurate and consistent for comparing peptides that have been tested on different days, or with different lots of purified MHC.

Because the antibody used for HLA-DR purification (LB3.1) is α-chain specific, β₁ molecules are not separated from β₃ (and/or β₄ and β₅) molecules. The β₁ specificity of the binding assay is obvious in the cases of DRB1*0101 (DR1), DRB1*0802 (DR8w2), and DRB1*0803 (DR8w3), where no β₃ is expressed. It has also been demonstrated for DRB1*0301 (DR3) and DRB3*0101 (DR52a), DRB1*0401 (DR4w4), DRB1*0404 (DR4w14), DRB1*0405 (DR4w15), DRB1*1101 (DR5), DRB1*1201 (DRw12), DRB1*1302 (DR6w19) and DRB1*0701 (DR7). The problem of β chain specificity for DRB1*1501 (DR2w2β₁), DRB5*0101 (DP2w2β₂), DRB1*1601 (DR2w21β₁), DRB5*0201 (DR51Dw21), and DRB4*0101 (DRw53) assays is circumvented by the use of fibroblasts. Development and validation of assays with regard to DRβ molecule specificity have been described previously (see, e.g., Southwood et al., J. Immunol. 160:3363-3373, 1998).

Binding assays as outlined above may be used to analyze supermotif and/or motif-bearing epitopes.

### Example 2

### Recognition of Variant Peptides by CTL Derived from DNA Immunization

Variants corresponding to five HLA-A2 and -A3 restricted epitopes from 167 HIV varianst were identified and synthesized. These represented all the complete sequences in the Los Alamos database at the time (116 strains), as well as 51 complete clade C sequences from Botswana, and included 22 subtype B and 62 subtype C sequences. These peptides were then characterized with regard to MHC binding, variant distribution, and immunogenicity. To measure immunogenicity, HLA-A2/K^{b} or HLA-A11/K^{b} transgenic mice were immunized with the epitopes encoded in a DNA based format ( ). Eleven days after immunization, splenocytes were restimulated with either the epitope corresponding to the epitope encoded by the DNA (parent) or each of the variant peptides. After 6 days in culture, IFN-γ secretion was measured in response to the peptide used to stimulate each culture.

The data for these epitopes are shown in FIG. 1. The HLA-A2-restricted epitope corresponding to the Env 134 epitope (KLTPLCVTL; FIG. 1A) used as the immunogen was the form observed most often (134/167). All single anchor variants were recognized to approximately the same extent as the parent peptide. Many of the single non-anchor variants (9/13) were also recognized within 10-fold of the parent peptide. Conservative substitutions (R and Q for K; see Table 4) at position 1 (P1) were tolerated, while the non-conservative substitution (E for K; see Table 4) lowered binding and eliminated recognition. Three P4 variants were observed. Two of these (F or S for P) were recognized within 10-fold of the recognition of the parent peptide, while one substitution (Q for P) completely eliminated recognition. The binding for these peptides was not significantly different from the parent peptide, indicating that this residue may be involved in TCR recognition. Both the conservative (F for L) and non-conservative (R for L) substitutions seen at P5 completely abrogated recognition, indicating that this residue is important in TCR recognition. Finally, one substitution at P8 (I for V), and four substitutions at P9 show little effect on recognition. None of the variants with multiple substitutions were recognized, although this may be due to the poor binding of these peptides.

The Gag 386 sequence utilized as the immunogen was the second most common form (VLAEAMSQV), present in 54 strains (FIG. 1B). The most prevalent variant, differing by a single tolerated C terminal anchor residue (V to A; 67 strains), was recognized equally to the parent epitope by CTL raised against the parent, as were the remaining single-anchor variants. Single substitutions were also tolerated at the non-anchor positions, P1 (I for V) and P8 (R, K, or H for Q). Only the P7 variant (G for S), probably a TCR contact residue, was not recognized.

Many of the multiple variants for Gag 386 were also recognized by CTL raised against the parent peptide. All the variants with multiple changes combined a change of V to A or T at the C terminus with 1-3 additional substitutions. Two variants with N terminal changes (V to A or I) were observed. The non-conservative A substitution was not recognized, while the conservative I substitution was. A double variant with a conservative substitution at P3 (A to G) was not recognized, implicating P3 in TCR recognition. Double variants with conservative changes at position 8 (Q to R, K, or H) were not well recognized, although the variants with single changes at the same positions were recognized. The variant combining a non-conservative A residue at position 8 with A at the C terminus was recognized as well as the parent. Equally surprising was the observation that all the variants with 3 or 4 substitutions were recognized within 10-fold of the parent peptide.

The parent form of the HLA-A2-restricted epitope, Vpr 62 (RILQQLLFI; FIG. 1C) was the most common form observed (86/167). Seven well-tolerated single anchor substitutions, 4 P2 and 3 C terminal, were also observed, accounting for most of the remaining variants (47/167). Single substitutions were, in general, also well tolerated. The single exception was the non-conservative substitution (P for L) at P6, while an M for L substitution at the same site was well tolerated. Binding was not affected for either variant, indicating that the reduction in activity is due to a change in a contact residue. Most variants with multiple changes also showed recognition to approximately the same extent as the parent. Several variants however did show reduced recognition. The variant with changes at both anchors (I to T at P2 and I to T at P9) had reduced binding (IC₅₀ of 9700), and recognition of the peptide was reduced, although not lost completely. Two variants with Q to H changes at P5, in combination with anchor residue changes (I to M at P2 and I to A at P9), exhibited greatly reduced recognition although binding was not affected. Other changes at P5 (Q to R or L at P5) reduced recognition only slightly.

The HLA-A3/11-restricted epitope, Pol 98 (FIG. 1D), represented the most diverse epitope in terms of the number of variant epitopes identified. The peptide encoded in the DNA was represented in only 18 out of 167 strains. Approximately a third of the peptides identified at that position (49 out of 167) did not have recognizable A3/A11 motifs. The most common variant (30 strains) differed from the parent peptide at 3 residues (VSIKVGGQIK), but was recognized within 10-fold of the parent peptide. Two variants with conservative changes at anchor residues were both recognized, although the T to A substitution at P2 resulted in a 10-fold reduction in recognition of the variant peptide. All peptides with single changes in non-anchor positions were also recognized, although the P5 variant (G to E) exhibited a decrease in recognition. As the binding was not affected, this probably indicates involvement in T cell recognition.

Peptides with two changes showed mixed results. In general, peptides with a V substitution at position 3, in combination with another substitution were recognized to the same extent as the corresponding single substitution, indicating the V substitution was tolerated well and is not a TCR contact residue. Combinations including the P2 anchor residue (T to A or N) were not recognized, although the binding of these peptides was also low. Variants with 3 substitutions were generally not recognized well. Two exceptions with very conservative substitutions were noted (FIG. 1D). CTL were unable to recognize peptides with four or more substitutions.

The HLA-A3/11-restricted Env 47 epitope (FIG. 1E; VTVYYGVPVWK) was highly conserved, with only 9 variants identified. The most common form observed was the parent peptide (99 strains), while the second most common form, a single anchor substitution observed in 40 strains, was recognized to the same extent as the parent. All the variants were recognized within 10-fold of the parent epitope.

Taken together, these data show trends towards promiscuous recognition of variant peptides by CTL generated from immunization with a single peptide. In general, changes that disrupted binding also decreased recognition. Recognition was also affected by the position of the change, with potential TCR contact residues (P3-7) exerting a greater effect on recognition than other residues. In general, conservative residue changes were more widely tolerated than were non-conservative changes. Recognition was also dependent on the number of changes, with progressively lower recognition with a greater number of changes.

Recognition after multiple restimulations The observed recognition of variant peptides by CTL raised against the parent peptide might be due to either promiscuous recognition at the level of a single TCR or simply a mixture of TCRs against the immunizing peptide which are each able to recognize subtly different peptides. To distinguish between these two possibilities, Env 134- or Gag 386-specific T cell lines were generated by stimulating five times with the immunizing peptide, and then tested for recognition of a partial panel of variant peptides. These T cell lines were also characterized for Vβ TCR usage against a panel of antibodies predicted to react with the TCR of the mouse strains utilized for these experiments.

The data for these peptide-specific lines are shown in Table 5. Because the SU is a measure of the number of cells needed to secrete a defined amount of IFN-γ, a higher SU value would correspond to an enrichment of IFN-γ, producing cells. A comparison of one and five peptide stimulations indeed shows an enrichment of CTL specific for the immunizing peptide for both of the peptide lines generated (Table 5A and 5B, first line). The Gag 386 line (Table 5A) also demonstrated increased recognition of all the variant peptides measured except one peptide (ILAEAMSKA) that was never recognized. The Env 134 line also demonstrated enrichment for CTL able to recognize several of the variant peptides (Table 5B).

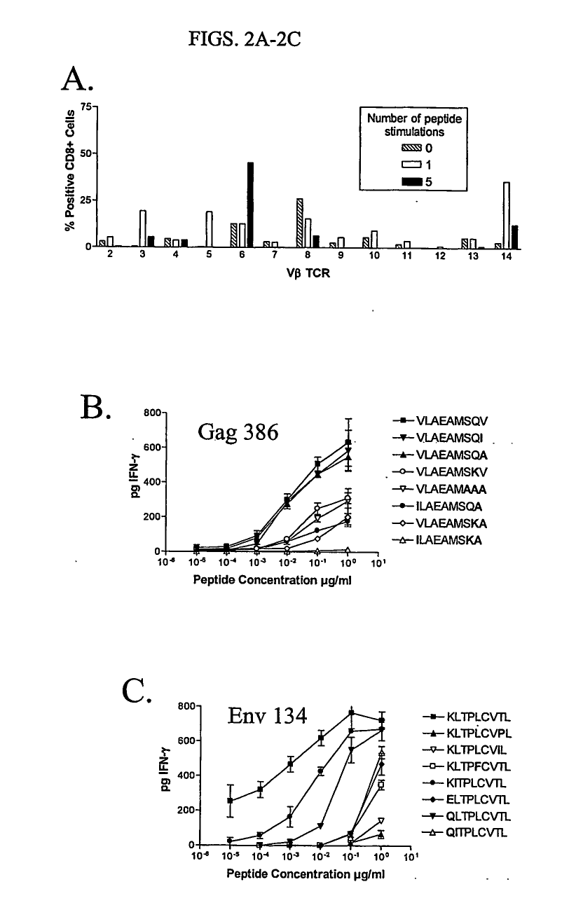

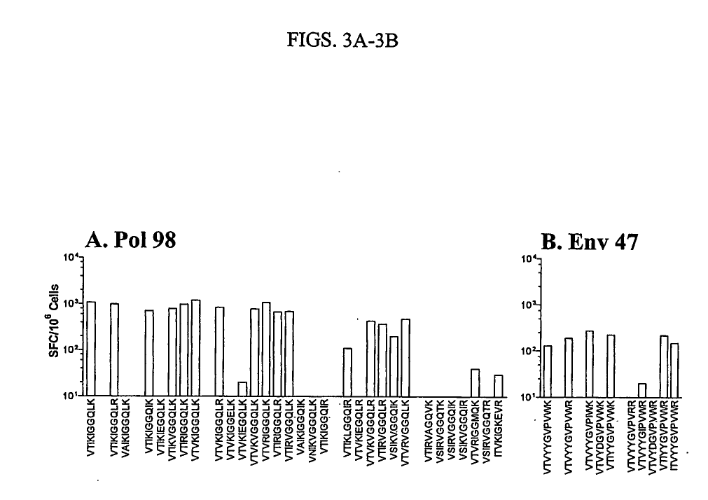

To further characterize these lines, we examined them for Vβ usage, utilizing a panel of commercially available antibodies available for mouse TCR Vβ 2-14. To determine background levels for the various TCR Vβ molecules, primary splenocytes from mice that had been immunized with EP HIV-1090 were also examined. The results for the Gag 386 line are shown in FIG. 2A. After a single stimulation with the parent peptide, the Gag 386 line showed a mixture of TCR positive populations, including Vβ 3, 5, and 14. After 5 stimulations, those populations had been reduced to background levels, and approximately 50% of the CD8+ cells expressed the Vβ 6 TCR. The Env 134 line showed a similar pattern of multiple TCR positive populations after a single round of stimulation with reduction to background levels after 5 stimulations (data not shown). However, no single Vβ usage significantly above background could be demonstrated, probably due to lack of the relevant TCR Vβ antibody.

Both lines were also characterized with regard to the affinity of certain of the variant peptides by titrating the variant peptides examined above (Table 5A and 5B). The data for both the Gag 386 and Env 134 lines are shown in FIG. 2B. For the Gag 386 line, the parent peptide along with two single anchor variants (VLAEAMSQI and VLAEAMSQA) showed the highest affinity. Four other peptides demonstrated lower affinity, but still produced IFN-γ in response to higher peptide concentrations. A single peptide (ILAEAMSKA) was not recognized.

As expected, the parent peptide, which was used to generate the Env 134 line, showed the highest affinity for the TCR. The other 2 variant peptides, KITPLCVTL and QLTPLCVTL, also demonstrated higher affinity, but reduced from the parent peptide by approximately 10-fold and 100-fold, respectively. It was notable that only at the highest peptide concentration examined (1 μg/ml) was any IFN-γ secretion detected for five of the peptides (QITPLCVTL, ELTPLCVTL, KLTPFCVTL, KLTPLCVIL, and KLTPLCVPL). These five peptides showed little or no enrichment of CTL able to recognize them, and exhibited the lowest activity as measured by SU after five restimulations (see Table 5B).

In summary, these cell lines seem to consist of a narrow, possibly single, TCR population. This TCR population recognizes the parent peptide with the highest affinity, but is also able to recognize a number of other variant peptides with equal or lesser affinity.

Recognition of Variant Peptides by CTL Derived from an HIV Infected Patient.

To determine if the same immunological conservation was observed in natural infections, we identified an HIV-infected individual expressing the HLA-A3 allele. The HIV strain and subtype with which this patient was infected is unknown. We had previously shown that T cells from this individual responded to the HLA-A3 restricted epitopes Pol 98 and Env 47. PBL from this patient were examined in an ELISPOT assay to determine if they also showed the capacity for broad cross-reactivity. The data are shown in FIG. 3. Although the actual peptide represented in the HIV strain with which this individual is infected is unknown, we observed recognition of a large number of the variant peptides for both Pol 98 (FIG. 3A) and Env 47 (FIG. 3B). The recognition patterns were remarkably similar for the mouse and patient data (compare FIG. 1 and FIG. 3), although the mouse expressed a transgene for HLA-A11 and the patient was HLA-A3.

Prediction of Immunological Conservation. We had observed that the variant peptides that were recognized by CTL raised against the parent epitope had amino acid substitutions that followed previous observations. For example, the anchor residue changes that were tolerated in the variant peptides were also described as anchors that to define the respective HLA supertypes ( ). In general, conservative substitutions were tolerated at non-anchor residues, while non-conservative substitutions were less well tolerated. These followed closely the prediction model used to identify heteroclitic analogs (Tangri et al).

Based on these observations, we designed a computer program to predict immunological conservation. For anchor positions, this program utilized the conserved anchor residues described for the A2, A3, and B7 supertypes. For non-anchor positions only conservative substitutions, as defined in Tangri et. al. ( ), were allowed. All substitutions at non-anchor positions were analyzed independently and all conservative substitutions were allowed regardless of the number of substitutions. Finally, the position of the substitution was not factored into analysis. Each variant was compared with the parent epitope, and its ability to be recognized was predicted as either positive or negative.

The first sets of epitopes to be evaluated by this program were the five HIV epitopes and variants previously described. For the Env 134 epitope, the program predicted that 13 of the variant peptides should be immunologically conserved, while 6 should not be recognized. Comparison of the observed immunological data with the prediction showed that the program predicted correctly for 14 of the peptides and incorrectly for 5. Of the incorrect predictions, in two cases the program predicted negative results for peptides that were recognized, while in 3 cases the program predicted positive results for peptides that were not recognized. A similar analysis was performed for all five peptides. Of 101 total variant peptides, 68 were correctly identified (67%). The discordant data were fairly evenly split between peptides incorrectly predicted negative (15) and those incorrectly predicted positive (18).

As noted previously, the more substitutions present in a variant peptide, the lower the likelihood of its immunogenicity. Since the prediction program treated all substitutions independently, and did not take into account the number of substitutions, we hypothesized that prediction of single substitutions would be more accurate. Indeed, the immunogenicity of 38 of 47 single substitution variants (80%) was correctly predicted.

With the limitations of the program in mind, it is useful to predict the recognition of the variants for a package of HLA-A2, -A3, and -B7 supertype epitopes. These epitopes had been identified as being well conserved in Clade B variants. When comparing the conservation of this group of epitopes based on sequence identity versus immunological conservation, it is interesting to note that the predicted recognition gains taking into account immunological conservation are significant (Table 6).

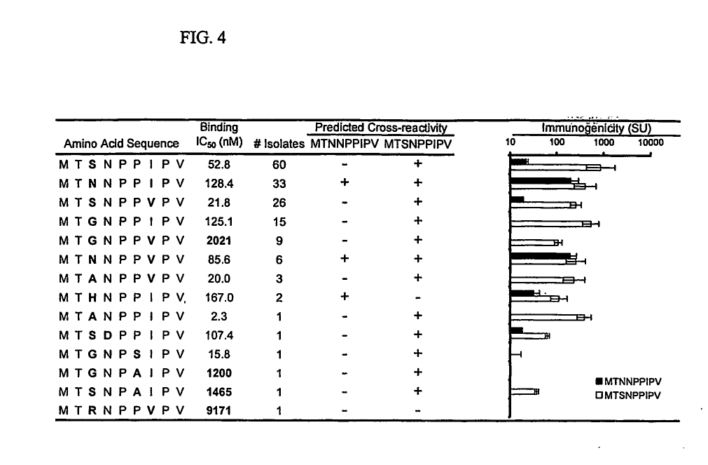

This particular group of 21 epitopes was selected based on their identity conservation in Clade B HIV sequences, with conservation across HIV clades as a secondary consideration. Because of this criteria, the form of epitope chosen as the parent peptide was not the most common variant (e.g. Gag 386, Gag 271, Pol 98). In some cases (e.g., see Gag 386 data), the “parent” epitope and the most common variant were recognized to the same extent. However, in some cases the selection of epitope to include as the “parent” epitope was predicted to make a difference in the immunological conservation. An example of this was the Gag 271 epitope (FIG. 4). The variant most commonly seen in clade B sequences was the MTNNPPIPV form, while the most common form of the epitope was MTSNPPIPV. Not all amino acids are considered equal to each other in their ability to substitute (Tangri). For example, asparagine (N) is considered a conservative substitution for serine (S), while the opposite substitution in only considered semi-conserved. When the program calculated immunological conservation using the MTNNPPIPV peptide as the parent peptide, only two variants were predicted to be immunogenic. However, when the immunological conservation was predicted using the MTSNPPIPV peptide, most of the variants were predicted to be recognized (FIG. 4). This prediction was tested using HLA-A2 transgenic mice. The results show that if the MTSNPPIPV form of the peptide was utilized in vaccines, approximately 152 of 167 variants would be recognized, while if the MTNNPPIPV form of the epitope was utilized, only 39 of 167 variants would be recognized. This has important implications in epitope selection for vaccine development, and epitope performance can be predicted.

### Example 3

### A Padre® Molecule as a Helper Epitope for Enhancement of CTL Induction

There is increasing evidence that HTL activity is critical for the induction of long lasting CTL responses (Livingston et al. J. Immunol 162:3088-3095 (1999); Walter et al., New Engl. J. Med. 333:1038-1044 (1995); Hu et al., J. Exp. Med. 177:1681-1690 (1993)). Therefore, one or more peptides that bind to HLA class II molecules and stimulate HTLs can be used in accordance with the invention. Accordingly, a preferred embodiment of a vaccine includes a molecule from the PADRE® family of universal T helper cell epitopes (HTL) that target most DR molecules in a manner designed to stimulate helper T cells. For instance, a pan-DR-binding epitope peptide having the formula: aKXVAAZTLKAAa, where “X” is either cyclohexylalanine, phenylalanine, or tyrosine; “Z” is either tryptophan, tyrosine, histidine or asparagine; and “a” is either D-alanine or L-alanine (SEQ ID NO:29), has been found to bind to most HLA-DR alleles, and to stimulate the response of T helper lymphocytes from most individuals, regardless of their HLA type.

A particularly preferred PADRE® molecule is a synthetic peptide, aKXVAAWTLKAAa (a=D-alanine, X=cyclohexylalanine), containing non-natural amino acids, specifically engineered to maximize both HLA-DR binding capacity and induction of T cell immune responses.

Alternative preferred PADRE® molecules are the peptides, aKFVAAWTLKAAa, aKYVAAWTLKAAa, aKFVAAYTLKAAa, aKXVAAYTLKAAa, aKYVAAYTLKAAa, aKFVAAHTLKAAa, aKXVAAHTLKAAa, aKYVAAHTLKAAa, aKFVAANTLKAAa, aKXVAANTLKAAa, aKYVAANTLKAAa, AKXVAAWTLKAAA (SEQ ID NO:30), AKFVAAWTLKAAA (SEQ ID NO:31), AKYVAAWTLKAAA (SEQ ID NO:32), AKFVAAYTLKAAA (SEQ ID NO:33), AKXVAAYTLKAAA (SEQ ID NO:34), AKYVAAYTLKAAA (SEQ ID NO:35), AKFVAAHTLKAAA (SEQ ID NO:36), AKXVAAHTLKAAA (SEQ ID NO:37), AKYVAAHTLKAAA (SEQ ID NO:38), AKFVAANTLKAAA (SEQ ID NO:39), AKXVAANTLKAAA (SEQ ID NO:40), AKYVAANTLKAAA (SEQ ID NO:41) (a=D-alanine, X=cyclohexylalanine).

In a preferred embodiment, the PADRE® peptide is amidated. For example, a particularly preferred amidated embodiment of a PADRE® molecule is conventionally written aKXVAAWTLKAAa-NH₂.

Competitive inhibition assays with purified HLA-DR molecules demonstrated that the PADRE® molecule aKXVAAWTLKAAa-NH₂ binds with high or intermediate affinity (IC₅₀≦1,000 nM) to 15 out of 16 of the most prevalent HLA-DR molecules ((Kawashima et al., Human Immunology 59:1-14 (1998); Alexander et al., Immunity 1:751-761 (1994)). A comparison of the DR binding capacity of PADRE® and tetanus toxoid (TT) peptide 830-843, a “universal” epitope has been published (Panina-Bordignon et al, Eur. J. Immunology 19:2237-2242 (1989)). The TT 830-843 peptide bound to only seven of 16 DR molecules tested, while PADRE® bound 15 of 16. At least 1 of the 15 DR molecules that bind PADRE® is predicted to be present in >95% of all humans. Therefore, this PADRE® molecule is anticipated to induce an HTL response in virtually all patients, despite the extensive polymorphism of HLA-DR molecules in the human population.

PADRE® has been specifically engineered for optimal immunogenicity for human T cells. Representative data from in vitro primary immunizations of normal human T cells with TT 830-843 antigen and the PADRE® molecule aKXVAAWTLKAAa-NH₂ are shown in FIG. 1. Peripheral blood mononuclear cells (PBMC) from three normal donors were stimulated with the peptides in vitro. Following the third round of stimulation, it was observed that PADRE® generated significant primary T cell responses for all three donors as measured in a standard T cell proliferation assay. With the PADRE® peptide, the 10,000 cpm proliferation level was generally reached with 10 to 100 ng/ml of antigen. In contrast, TT 830-843 antigen generated responses for only 2 out of 3 of the individuals tested. Responses approaching the 10,000 cpm range were reached with about 10,000 ng/ml of antigen. In this respect, it was noted that PADRE® was, on a molar basis, about 100-fold more potent than TT 830-843 antigen for activation of T cell responses.

Early data from a phase I/II investigator-sponsored trial, conducted at the University of Leiden (C. J. M. Melief), support the principle that the PADRE® molecule aKXVAAWTLKAAa, possibly the amidated aKXVAAWTLKAAa —NH₂, is highly immunogenic in humans (Ressing et al., J. Immunother. 23(2):255-66 (2000)). In this trial, a PADRE® molecule was co-emulsified with various human papilloma virus (HPV)-derived CTL epitopes and was injected into patients with recurrent or residual cervical carcinoma. However, because of the late stage of carcinoma with the study patients, it was expected that these patients were immunocompromised. The patients' immuno compromised status was demonstrated by their low frequency of influenza virus-specific CTL, reduced levels of CD3 expression, and low incidence of proliferative recall responses after in vitro stimulation with conventional antigens. Thus, no efficacy was anticipated in the University of Leiden trial, rather the goal of that trial was essentially to evaluate safety. Safety was, in fact, demonstrated. In addition to a favorable safety profile, PADRE® T cell reactivity was detected in four of 12 patients (FIG. 2) in spite of the reduced immune competence of these patients.

Thus, the PADRE® peptide component(s) of the vaccine bind with broad specificity to multiple allelic forms of HLA-DR molecules. Moreover, PADRE® peptide component(s) bind with high affinity (IC₅₀≦1000 nM), i.e., at a level of affinity correlated with being immunogenic for HLA Class II restricted T cells. The in vivo administration of PADRE® peptide(s) stimulates the proliferation of HTL in normal humans as well as patient populations.

One or more PADRE® peptide(s) may be included in a composition, e.g., a vaccine, comprising one or more peptides, either as an individual peptide(s), fused to one or more variant peptides, or both.

### Example 4

### CTL Recognition of Endogenous Processed Antigens after Priming

This example determines that CTL induced by native or analoged peptide epitopes recognize endogenously synthesized, i.e., native antigens.

Effector cells isolated from transgenic mice that are immunized with peptide epitopes are re-stimulated in vitro using peptide-coated stimulator cells. Six days later, effector cells are assayed for cytotoxicity and the cell lines that contain peptide-specific cytotoxic activity are further re-stimulated. An additional six days later, these cell lines are tested for cytotoxic activity on ⁵¹Cr labeled Jurkat-A2.1/K^{b} target cells in the absence or presence of peptide, and also tested on ⁵¹Cr labeled target cells bearing the endogenously synthesized antigen, i.e. cells that are stably transfected with HIV expression vectors.

The result will demonstrate that CTL lines obtained from animals primed with peptide epitope recognize endogenously synthesized HIV antigen. The choice of transgenic mouse model to be used for such an analysis depends upon the epitope(s) that is being evaluated. In addition to HLA-A*0201/K^{b} transgenic mice, several other transgenic mouse models including mice with human A11, which may also be used to evaluate A3 epitopes, and B7 alleles have been characterized and others (e.g., transgenic mice for HLA-A1 and A24) are being developed. HLA-DR1 and HLA-DR3 mouse models have also been developed, which may be used to evaluate HTL epitopes.

### Example 5

### Activity of CTL-HTL Conjugated Epitopes in Transgenic Mice

This example illustrates the induction of CTLs and HTLs in transgenic mice by use of a HIV CTL/HTL peptide conjugate whereby the vaccine composition comprises peptides administered to an HIV-infected patient or an individual at risk for HIV. The peptide composition can comprise multiple CTL and/or HTL epitopes. This analysis demonstrates enhanced immunogenicity that can be achieved by inclusion of one or more HTL epitopes in a vaccine composition. Such a peptide composition can comprise an HTL epitope conjugated to a preferred CTL epitope containing, for example, at least one CTL epitope, or an analog of that epitope. The peptides may be lipidated, if desired.

Immunization procedures: Immunization of transgenic mice is performed as described (Alexander et al., J. Immunol. 159:4753-4761, 1997). For example, A2/K^{b} mice, which are transgenic for the human HLA A2.1 allele and are useful for the assessment of the immunogenicity of HLA-A*0201 motif- or HLA-A2 supermotif-bearing epitopes, are primed subcutaneously (base of the tail) with a 0.1 ml of peptide in Incomplete Freund's Adjuvant, or if the peptide composition is a lipidated CTL/HTL conjugate, in DMSO/saline or if the peptide composition is a polypeptide, in PBS or Incomplete Freund's Adjuvant. Seven days after priming, splenocytes obtained from these animals are restimulated with syngenic irradiated LPS-activated lymphoblasts coated with peptide.

Cell lines: Target cells for peptide-specific cytotoxicity assays are Jurkat cells transfected with the HLA-A2.1/K^{b} chimeric gene (e.g., Vitiello et al., J. Exp. Med. 173:1007, 1991).

In vitro CTL activation: One week after priming, spleen cells (30×10⁶ cells/flask) are co-cultured at 37° C. with syngeneic, irradiated (3000 rads), peptide coated lymphoblasts (10×10⁶ cells/flask) in 10 ml of culture medium/T25 flask. After six days, effector cells are harvested and assayed for cytotoxic activity.

Assay for cytotoxic activity: Target cells (1.0 to 1.5×10⁶) are incubated at 37° C. in the presence of 200 μl of ⁵¹Cr. After 60 minutes, cells are washed three times and resuspended in R10 medium. Peptide is added where required at a concentration of 1 μg/ml. For the assay, 10^{4 51} Cr-labeled target cells are added to different concentrations of effector cells (final volume of 200 μl) in U-bottom 96-well plates. After a 6 hour incubation period at 37° C., a 0.1 ml aliquot of supernatant is removed from each well and radioactivity is determined in a Micromedic automatic gamma counter. The percent specific lysis is determined by the formula: percent specific release=100×(experimental release−spontaneous release)/(maximum release−spontaneous release). To facilitate comparison between separate CTL assays run under the same conditions, % ⁵¹Cr release data is expressed as lytic units/10⁶ cells. One lytic unit is arbitrarily defined as the number of effector cells required to achieve 30% lysis of 10,000 target cells in a 6 hour ⁵¹Cr release assay. To obtain specific lytic units/10⁶, the lytic units/10⁶ obtained in the absence of peptide is subtracted from the lytic units/10⁶ obtained in the presence of peptide. For example, if 30% ⁵¹Cr release is obtained at the effector (E): target (T) ratio of 50:1 (i.e., 5×10⁵ effector cells for 10,000 targets) in the absence of peptide and 5:1 (i.e., 5×10⁴ effector cells for 10,000 targets) in the presence of peptide, the specific lytic units would be: [( 1/50,000)−( 1/500,000)]×10⁶=18 LU.

The results are analyzed to assess the magnitude of the CTL responses of animals injected with the immunogenic CTL/HTL conjugate vaccine preparation and are compared to the magnitude of the CTL response achieved using the CTL epitope as outlined in above. Analyses similar to this may be performed to evaluate the immunogenicity of peptide conjugates containing multiple CTL epitopes and/or multiple HTL epitopes. In accordance with these procedures it is found that a CTL response is induced, and concomitantly that an HTL response is induced upon administration of such compositions.

### Example 6

### Selection of CTL and HTL Epitopes for Inclusion in an HIV-Specific Vaccine

This example illustrates the procedure for the selection of peptide epitopes for vaccine compositions of the invention. The peptides in the composition can be in the form of a nucleic acid sequence, either single or one or more sequences (i.e., minigene) that encodes peptide(s), or can be single and/or polyepitopic peptides.

The following principles are utilized when selecting an array of epitopes for inclusion in a vaccine composition. Each of the following principles is balanced in order to make the selection.

Epitopes are selected which, upon administration, mimic immune responses that correlate with virus clearance. For example, if it has been observed that patients who clear HIV generate an immune response to at least 3 epitopes on at least one HIV antigen, then 3-4 epitopes should be included for HLA class I. A similar rationale is used to determine HLA class II epitopes.

When selecting an array of HIV epitopes, it is preferred that at least some of the epitopes are derived from early and late proteins. The early proteins of HIV are expressed when the virus is replicating, either following acute or dormant infection. Therefore, it is particularly preferred to use epitopes from early stage proteins to alleviate disease manifestations at the earliest stage possible.

Epitopes are often selected that have a binding affinity of an IC₅₀ of 500 nM or less for an HLA class I molecule, or for class II, an IC₅₀ of 1000 nM or less.

Sufficient supermotif bearing peptides, or a sufficient array of allele-specific motif bearing peptides, are selected to give broad population coverage. For example, epitopes are selected to provide at least 80% population coverage. A Monte Carlo analysis, a statistical evaluation known in the art, can be employed to assess breadth, or redundancy, of population coverage.

When creating a polyepitopic compositions, e.g. a minigene, it is typically desirable to generate the smallest peptide possible that encompasses the epitopes of interest. The principles employed are similar, if not the same, as those employed when selecting a peptide comprising nested epitopes.

In cases where the sequences of multiple variants of the same target protein are available, potential peptide epitopes can also be selected on the basis of their conservancy. For example, a criterion for conservancy may define that the entire sequence of an HLA class I binding peptide or the entire 9-mer core of a class II binding peptide be conserved in a designated percentage of the sequences evaluated for a specific protein antigen.

Peptide epitopes for inclusion in vaccine compositions are, for example, selected from those listed in Tables 6-9 or FIGS. 1A-4. A vaccine composition comprised of selected peptides, when administered, is safe, efficacious, and elicits an immune response similar in magnitude of an immune response that clears an acute HIV infection.

### Example 7

### Construction of Minigene Multi-Epitope DNA Plasmids

This example provides general guidance for the construction of a minigene expression plasmid. Minigene plasmids may, of course, contain various configurations of CTL and/or HTL epitopes or epitope analogs as described herein. Expression plasmids have been constructed and evaluated as described, for example, in co-pending U.S. Ser. No. 09/311,784 filed May 13, 1999 and in Ishioka et al., J. Immunol. 162:3915-3925, 1999. An example of such a plasmid for the expression of HIV epitopes is shown in FIG. 2, which illustrates the orientation of HIV peptide epitopes in a minigene construct.

A minigene expression plasmid typically includes multiple CTL and HTL peptide epitopes. In the present example, HLA-A2, -A3, -B7 supermotif-bearing peptide epitopes and HLA-A1 and -A24 motif-bearing peptide epitopes are used in conjunction with DR supermotif-bearing epitopes and/or DR3 epitopes (FIG. 2). Preferred epitopes are identified, for example, in Tables 6-9 and FIGS. 1A-4. HLA class I supermotif or motif-bearing peptide epitopes derived from multiple HIV antigens, are selected such that multiple supermotifs/motifs are represented to ensure broad population coverage. Similarly, HLA class II epitopes are selected from multiple HIV antigens to provide broad population coverage, i.e. both HLA DR-1-4-7 supermotif-bearing epitopes and HLA DR-3 motif-bearing epitopes are selected for inclusion in the minigene construct. The selected CTL and HTL epitopes are then incorporated into a minigene for expression in an expression vector.

Such a construct may additionally include sequences that direct the HTL epitopes to the endoplasmic reticulum. For example, the Ii protein may be fused to one or more HTL epitopes as described in co-pending application U.S. Ser. No. 09/311,784 filed May 13, 1999, wherein the CLIP sequence of the Ii protein is removed and replaced with an HLA class II epitope sequence os that HLA class II epitope is directed to the endoplasmic reticulum, where the epitope binds to an HLA class II molecules.

This example illustrates the methods to be used for construction of a minigene-bearing expression plasmid. Other expression vectors that may be used for minigene compositions are available and known to those of skill in the art.

The minigene DNA plasmid contains a consensus Kozak sequence and a consensus murine kappa Ig-light chain signal sequence followed by CTL and/or HTL epitopes selected in accordance with principles disclosed herein. The construct can also include, for example, The sequence encodes an open reading frame fused to the Myc and His antibody epitope tag coded for by the pcDNA 3.1 Myc-His vector.

Overlapping oligonucleotides, for example eight oligonucleotides, averaging approximately 70 nucleotides in length with 15 nucleotide overlaps, are synthesized and HPLC-purified. The oligonucleotides encode the selected peptide epitopes as well as appropriate linker nucleotides, Kozak sequence, and signal sequence. The final multiepitope minigene is assembled by extending the overlapping oligonucleotides in three sets of reactions using PCR. A Perkin/Elmer 9600 PCR machine is used and a total of 30 cycles are performed using the following conditions: 95° C. for 15 sec, annealing temperature (5° below the lowest calculated Tm of each primer pair) for 30 sec, and 72° C. for 1 min.

For the first PCR reaction, 5 μg of each of two oligonucleotides are annealed and extended: Oligonucleotides 1+2, 3+4, 5+6, and 7+8 are combined in 100 μl reactions containing Pfu polymerase buffer (1×=10 mM KCL, 10 mM (NH₄)₂SO₄, 20 mM Tris-chloride, pH 8.75, 2 mM MgSO₄, 0.1% Triton X-100, 100 μg/ml BSA), 0.25 mM each dNTP, and 2.5 U of Pfu polymerase. The full-length dimer products are gel-purified, and two reactions containing the product of 1+2 and 3+4, and the product of 5+6 and 7+8 are mixed, annealed, and extended for 10 cycles. Half of the two reactions are then mixed, and 5 cycles of annealing and extension carried out before flanking primers are added to amplify the full length product for 25 additional cycles. The full-length product is gel-purified and cloned into pCR-blunt (Invitrogen) and individual clones are screened by sequencing.

### Example 8

### The Plasmid Construct and the Degree to which it Induces Immunogenicity

The degree to which a plasmid construct, for example a plasmid constructed in accordance as above is able to induce immunogenicity can be evaluated in vitro by testing for epitope presentation by APC following transduction or transfection of the APC with an epitope-expressing nucleic acid construct. Such a study determines “antigenicity” and allows the use of human APC. The assay determines the ability of the epitope to be presented by the APC in a context that is recognized by a T cell by quantifying the density of epitope-HLA class I complexes on the cell surface. Quantitation can be performed by directly measuring the amount of peptide eluted from the APC (see, e.g., Sijts et al., J. Immunol. 156:683-692, 1996; Demotz et al., Nature 342:682-684, 1989); or the number of peptide-HLA class I complexes can be estimated by measuring the amount of lysis or lymphokine release induced by infected or transfected target cells, and then determining the concentration of peptide necessary to obtained equivalent levels of lysis or lymphokine release (see, e.g., Kageyama et al., J. Immunol. 154:567-576, 1995).

Atlernatively, immunogenicity can be evaluated through in vivo injections into mice and subsequent in vitro assessment of CTL and HTL activity, which are analysed using cytotoxicity and proliferation assays, respectively, as detailed e.g., in copending U.S. Ser. No. 09/311,784 filed May 13, 1999 and Alexander et al., Immunity 1:751-761, 1994.

For example, to assess the capacity of a DNA minigene construct (e.g., a pMin minigene construct generated as decribed in U.S. Ser. No. 09/311,784) containing at least one HLA-A2 supermotif peptide to induce CTLs in vivo, HLA-A2.1/K^{b} transgenic mice, for example, are immunized intramuscularly with 100 μg of naked cDNA. As a means of comparing the level of CTLs induced by cDNA immunization, a control group of animals is also immunized with an actual peptide composition that comprises multiple epitopes synthesized as a single polypeptide as they would be encoded by the minigene.

Splenocytes from immunized animals are stimulated twice with each of the respective compositions (peptide epitopes encoded in the minigene or the polyepitopic peptide), then assayed for peptide-specific cytotoxic activity in a ⁵¹Cr release assay. The results indicate the magnitude of the CTL response directed against the A2-restricted epitope, thus indicating the in vivo immunogenicity of the minigene vaccine and polyepitopic vaccine. It is, therefore, found that the minigene elicits immune responses directed toward the HLA-A2 supermotif peptide epitopes as does the polyepitopic peptide vaccine. A similar analysis is also performed using other HLA-A3 and HLA-B7 transgenic mouse models to assess CTL induction by HLA-A3 and HLA-B7 motif or supermotif epitopes.

To assess the capacity of a class II epitope encoding minigene to induce HTLs in vivo, DR transgenic mice, or for those epitope that cross react with the appropriate mouse MHC molecule, I-A^{b}-restricted mice, for example, are immunized intramuscularly with 100 μg of plasmid DNA. As a means of comparing the level of HTLs induced by DNA immunization, a group of control animals is also immunized with an actual peptide composition emulsified in complete Freund's adjuvant. CD4+ T cells, i.e. HTLs, are purified from splenocytes of immunized animals and stimulated with each of the respective compositions (peptides encoded in the minigene). The HTL response is measured using a ³H-thymidine incorporation proliferation assay, (see, e.g. Alexander et al. Immunity 1:751-761, 1994). The results indicate the magnitude of the HTL response, thus demonstrating the in vivo immunogenicity of the minigene.

DNA minigenes, constructed as described above or below, may also be evaluated as a vaccine in combination with a boosting agent using a prime boost protocol. The boosting agent can consist of recombinant protein (e.g. Barnett et al., Aids Res. and Human Reroviruses 14, Supplement 3:S299-S309, 1998) or recombinant vaccinia, for example, expressing a minigene or DNA encoding the complete protein of interest (see, e.g., Hanke et al., Vaccine 16:439-445, 1998; Sedegah et al., Proc. Natl. Acad. Sci USA 95:7648-53, 1998; Hanke and McMichael, Immunol. Letters 66:177-181, 1999; and Robinson et al., Nature Med. 5:526-34, 1999).

For example, the efficacy of the DNA minigene used in a prime boost protocol is initially evaluated in transgenic mice. In this example, A2.1/K^{b} transgenic mice are immunized IM with 100 μg of a DNA minigene encoding the immunogenic peptides including at least one HLA-A2 supermotif-bearing peptide. After an incubation period (ranging from 3-9 weeks), the mice are boosted IP with 10⁷ pfu/mouse of a recombinant vaccinia virus expressing the same sequence encoded by the DNA minigene. Control mice are immunized with 100 μg of DNA or recombinant vaccinia without the minigene sequence, or with DNA encoding the minigene, but without the vaccinia boost. After an additional incubation period of two weeks, splenocytes from the mice are immediately assayed for peptide-specific activity in an ELISPOT assay. Additionally, splenocytes are stimulated in vitro with the A2-restricted peptide epitopes encoded in the minigene and recombinant vaccinia, then assayed for peptide-specific activity in an IFN-γ ELISA.

It is found that the minigene utilized in a prime-boost protocol elicits greater immune responses toward the HLA-A2 supermotif peptides than with DNA alone. Such an analysis can also be performed using HLA-A11 or HLA-B7 transgenic mouse models to assess CTL induction by HLA-A3 or HLA-B7 motif or supermotif epitopes.

The use of prime boost protocols in humans is described in below.

### Example 9

### Peptide Composition for Prophylactic Uses

Vaccine compositions of the present invention can be used to prevent HIV infection in persons who are at risk for such infection. For example, a polyepitopic peptide epitope composition (or a nucleic acid comprising the same) containing multiple CTL and HTL epitopes, which are also selected to target greater than 80% of the population, is administered to individuals at risk for HIV infection.

For example, a peptide-based composition can be provided as a single polypeptide that encompasses multiple epitopes. The vaccine is typically administered in a physiological solution that comprises an adjuvant, such as Incomplete Freunds Adjuvant. The dose of peptide for the initial immunization is from about 1 to about 50,000 μg, generally 100-5,000 μg, for a 70 kg patient. The initial administration of vaccine is followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious as a prophylaxis against HIV infection.

Alternatively, a composition typically comprising transfecting agents can be used for the administration of a nucleic acid-based vaccine in accordance with methodologies known in the art and disclosed herein.

### Example 10

### Polyepitopic Vaccine Compositions Derived from Native HIV Sequences

A native HIV polyprotein sequence is screened, preferably using computer algorithms defined for each class I and/or class II supermotif or motif, to identify “relatively short” regions of the polyprotein that comprise multiple epitopes and is preferably less in length than an entire native antigen. This relatively short sequence that contains multiple distinct, even overlapping, epitopes is selected and used to generate a minigene construct. The construct is engineered to express the peptide, which corresponds to the native protein sequence. The “relatively short” peptide is generally less than 250 amino acids in length, often less than 100 amino acids in length, preferably less than 75 amino acids in length, and more preferably less than 50 amino acids in length. The protein sequence of the vaccine composition is selected because it has maximal number of epitopes contained within the sequence, i.e., it has a high concentration of epitopes. As noted herein, epitope motifs may be nested or overlapping, for example, two 9-mer epitopes and one 10-mer epitope can be present in a 10 amino acid peptide. Such a vaccine composition is administered for therapeutic or prophylactic purposes.

The vaccine composition will preferably include, for example, three CTL epitopes and at least one HTL epitope from HIV. This polyepitopic native sequence is administered either as a peptide or as a nucleic acid sequence which encodes the peptide. Alternatively, an analog can be made of this native sequence, whereby one or more of the epitopes comprise substitutions that alter the cross-reactivity and/or binding affinity properties of the polyepitopic peptide.

The embodiment of this example provides for the possibility that an as yet undiscovered aspect of immune system processing will apply to the native nested sequence and thereby facilitate the production of therapeutic or prophylactic immune response-inducing vaccine compositions. Additionally such an embodiment provides for the possibility of motif-bearing epitopes for an HLA makeup that is presently unknown. Furthermore, this embodiment (absent analogs) directs the immune response to multiple peptide sequences that are actually present in native HIV antigens thus avoiding the need to evaluate any junctional epitopes. Lastly, the embodiment provides an economy of scale when producing nucleic acid vaccine compositions.

Related to this embodiment, computer programs can be derived in accordance with principles in the art, which identify in a target sequence, the greatest number of epitopes per sequence length.

### Example 11

### Polyepitopic Vaccine Compositions Directed to Multiple Diseases

The HIV peptide epitopes of the present invention are used in conjunction with peptide epitopes from target antigens related to one or more other diseases, to create a vaccine composition that is useful for the prevention or treatment of HIV as well as the one or more other disease(s). Examples of the other diseases include, but are not limited to, HCV and HBV.

For example, a polyepitopic peptide composition comprising multiple CTL and HTL epitopes that target greater than 98% of the population may be created for administration to individuals at risk for both HBV and HIV infection. The composition can be provided as a single polypeptide that incorporates the multiple epitopes from the various disease-associated sources, or can be administered as a composition comprising one or more discrete epitopes.

### Example 12

### Use of Peptides to Evaluate an Immune Response

Peptides of the invention may be used to analyze an immune response for the presence of specific CTL or HTL populations directed to HIV. Such an analysis may be performed in a manner as that described by Ogg et al., Science 279:2103-2106, 1998. In the following example, peptides in accordance with the invention are used as a reagent for diagnostic or prognostic purposes, not as an immunogen.

In this example highly sensitive human leukocyte antigen tetrameric complexes (“tetramers”) are used for a cross-sectional analysis of, for example, HIV HLA-A*0201-specific CTL frequencies from HLA A*0201-positive individuals at different stages of infection or following immunization using an HIV peptide containing an A*0201 motif Tetrameric complexes are synthesized as described (Musey et al., N. Engl. J. Med. 337:1267, 1997). Briefly, purified HLA heavy chain (A*0201 in this example) and β2-microglobulin are synthesized by means of a prokaryotic expression system. The heavy chain is modified by deletion of the transmembrane-cytosolic tail and COOH-terminal addition of a sequence containing a BirA enzymatic biotinylation site. The heavy chain, β2-microglobulin, and peptide are refolded by dilution. The 45-kD refolded product is isolated by fast protein liquid chromatography and then biotinylated by BirA in the presence of biotin. (Sigma, St. Louis, Mo.), adenosine 5′triphosphate and magnesium. Streptavidin-phycoerythrin conjugate is added in a 1:4 molar ratio, and the tetrameric product is concentrated to 1 mg/ml. The resulting product is referred to as tetramer-phycoerythrin.

For the analysis of patient blood samples, approximately one million PBMCs are centrifuged at 300×g for 5 minutes and resuspended in 50 pd of cold phosphate-buffered saline. Tri-color analysis is performed with the tetramer-phycoerythrin, along with anti-CD8-Tricolor, and anti-CD38. The PBMCs are incubated with tetramer and antibodies on ice for 30 to 60 min and then washed twice before formaldehyde fixation. Gates are applied to contain >99.98% of control samples. Controls for the tetramers include both A*0201-negative individuals and A*0201-positive uninfected donors. The percentage of cells stained with the tetramer is then determined by flow cytometry. The results indicate the number of cells in the PBMC sample that contain epitope-restricted CTLs, thereby readily indicating the extent of immune response to the HIV epitope, and thus the stage of infection with HIV, the status of exposure to HIV, or exposure to a vaccine that elicits a protective or therapeutic response.

### Example 13

### Use of Peptide Epitopes to Evaluate Recall Responses

The peptide epitopes of the invention are used as reagents to evaluate T cell responses, such as acute or recall responses, in patients. Such an analysis may be performed on patients who have recovered from infection, who are chronically infected with HIV, or who have been vaccinated with an HIV vaccine.

For example, the class I restricted CTL response of persons who have been vaccinated may be analyzed. The vaccine may be any HIV vaccine. PBMC are collected from vaccinated individuals and HLA typed. Appropriate peptide epitopes of the invention that, optimally, bear supermotifs to provide cross-reactivity with multiple HLA supertype family members, are then used for analysis of samples derived from individuals who bear that HLA type.

PBMC from vaccinated individuals are separated on Ficoll-Histopaque density gradients (Sigma Chemical Co., St. Louis, Mo.), washed three times in HBSS (GIBCO Laboratories), resuspended in RPMI-1640 (GIBCO Laboratories) supplemented with L-glutamine (2 mM), penicillin (50 U/ml), streptomycin (50 μg/ml), and Hepes (10 mM) containing 10% heat-inactivated human AB serum (complete RPMI) and plated using microculture formats. A synthetic peptide comprising an epitope of the invention is added at 10 μg/ml to each well and HBV core 128-140 epitope is added at 1 μg/ml to each well as a source of T cell help during the first week of stimulation.

In the microculture format, 4×10⁵ PBMC are stimulated with peptide in 8 replicate cultures in 96-well round bottom plate in 100 μl/well of complete RPMI. On days 3 and 10, 100 ml of complete RPMI and 20 U/ml final concentration of rIL-2 are added to each well. On day 7 the cultures are transferred into a 96-well flat-bottom plate and restimulated with peptide, rIL-2 and 10⁵ irradiated (3,000 rad) autologous feeder cells. The cultures are tested for cytotoxic activity on day 14. A positive CTL response requires two or more of the eight replicate cultures to display greater than 10% specific ⁵¹Cr release, based on comparison with uninfected control subjects as previously described (Rehermann, et al., Nature Med. 2:1104,1108, 1996; Rehermann et al., J. Clin. Invest. 97:1655-1665, 1996; and Rehermann et al. J. Clin. Invest. 98:1432-1440, 1996).

Target cell lines are autologous and allogeneic EBV-transformed B-LCL that are either purchased from the American Society for Histocompatibility and Immunogenetics (ASHI, Boston, Mass.) or established from the pool of patients as described (Guilhot, et al. J. Virol. 66:2670-2678, 1992).

Cytotoxicity assays are performed in the following manner. Target cells consist of either allogeneic HLA-matched or autologous EBV-transformed B lymphoblastoid cell line that are incubated overnight with the synthetic peptide epitope of the invention at 10 μM, and labeled with 100 μCi of ⁵¹Cr (Amersham Corp., Arlington Heights, Ill.) for 1 hour after which they are washed four times with HBSS.

Cytolytic activity is determined in a standard 4-h, split well ⁵¹Cr release assay using U-bottomed 96 well plates containing 3,000 targets/well. Stimulated PBMC are tested at effector/target (E/T) ratios of 20-50:1 on day 14. Percent cytotoxicity is determined from the formula: 100×[(experimental release-spontaneous release)/maximum release-spontaneous release)]. Maximum release is determined by lysis of targets by detergent (2% Triton X-100; Sigma Chemical Co., St. Louis, Mo.). Spontaneous release is <25% of maximum release for all experiments.

The results of such an analysis indicate the extent to which HLA-restricted CTL populations have been stimulated by previous exposure to HIV or an HIV vaccine.

The class II restricted HTL responses may also be analyzed. Purified PBMC are cultured in a 96-well flat bottom plate at a density of 1.5×10⁵ cells/well and are stimulated with 10 μg/ml synthetic peptide, whole antigen, or PHA. Cells are routinely plated in replicates of 4-6 wells for each condition. After seven days of culture, the medium is removed and replaced with fresh medium containing 10 U/ml IL-2. Two days later, 1 μCi ³H-thymidine is added to each well and incubation is continued for an additional 18 hours. Cellular DNA is then harvested on glass fiber mats and analyzed for ³H-thymidine incorporation. Antigen-specific T cell proliferation is calculated as the ratio of ³H-thymidine incorporation in the presence of antigen divided by the ³H-thymidine incorporation in the absence of antigen.

### Example 14

### Induction of Specific CTL Response in Humans

A human clinical trial for an immunogenic composition comprising CTL and HTL epitopes of the invention is set up as an IND Phase I, dose escalation study and carried out as a randomized, double-blind, placebo-controlled trial. Such a trial is designed, for example, as follows:

A total of about 27 subjects are enrolled and divided into 3 groups:

Group I: 3 subjects are injected with placebo and 6 subjects are injected with 5 μg of peptide composition;

Group II: 3 subjects are injected with placebo and 6 subjects are injected with 50 μg peptide composition;

Group III: 3 subjects are injected with placebo and 6 subjects are injected with 500 μg of peptide composition.

After 4 weeks following the first injection, all subjects receive a booster inoculation at the same dosage.

The endpoints measured in this study relate to the safety and tolerability of the peptide composition as well as its immunogenicity. Cellular immune responses to the peptide composition are an index of the intrinsic activity of this the peptide composition, and can therefore be viewed as a measure of biological efficacy. The following summarize the clinical and laboratory data that relate to safety and efficacy endpoints.

Safety: The incidence of adverse events is monitored in the placebo and drug treatment group and assessed in terms of degree and reversibility.

Evaluation of Vaccine Efficacy: For evaluation of vaccine efficacy, subjects are bled before and after injection. Peripheral blood mononuclear cells are isolated from fresh heparinized blood by Ficoll-Hypaque density gradient centrifugation, aliquoted in freezing media and stored frozen. Samples are assayed for CTL and HTL activity.

The vaccine is found to be both safe and efficacious.

### Example 15

### Phase II Trials in Patients Infected with HIV

Phase II trials are performed to study the effect of administering the CTL-HTL peptide compositions to HIV-infected patients. The main objectives of the trials are to determine an effective dose and regimen for inducing CTLs in chronically infected HIV patients, to establish the safety of inducing a CTL and HTL response in these patients, and to see to what extent activation of CTLs improves the clinical picture of chronically infected HIV patients, as manifested by a reduction in viral load and an increase in CD4⁺ cells counts. Such a study is designed, for example, as follows:

The studies are performed in multiple centers. The trial design is an open-label, uncontrolled, dose escalation protocol wherein the peptide composition is administered as a single dose followed six weeks later by a single booster shot of the same dose. The dosages are 50, 500 and 5,000 micrograms per injection. Drug-associated adverse effects (severity and reversibility) are recorded.

There are three patient groupings. The first group is injected with 50 micrograms of the peptide composition and the second and third groups with 500 and 5,000 micrograms of peptide composition, respectively. The patients within each group range in age from 21-65, include both males and females, and represent diverse ethnic backgrounds. All of them are infected with HIV for over five years and are HCV, HBV and delta hepatitis virus (HDV) negative, but have positive levels of HIV antigen.

The viral load and CD4⁺ levels are monitored to assess the effects of administering the peptide compositions. The vaccine composition is found to be both safe and efficacious in the treatment HIV infection.

### Example 16

### Induction of CTL Responses Using a Prime Boost Protocol

A prime boost protocol can also be used for the administration of the vaccine to humans. Such a vaccine regimen can include an initial administration of, for example, naked DNA followed by a boost using recombinant virus encoding the vaccine, or recombinant protein/polypeptide or a peptide mixture administered in an adjuvant.

For example, the initial, immunization is performed using an expression vector, such as that constructed above, in the form of naked nucleic acid administered IM (or SC or ID) in the amounts of 0.5-5 mg at multiple sites. The nucleic acid (0.1 to 1000 μg) can also be administered using a gene gun. Following an incubation period of 3-4 weeks, a booster dose is then administered. The booster is, for example, recombinant fowlpox virus administered at a dose of 5-10⁷ to 5×10⁹ pfu. An alternative recombinant virus, such as an MVA, canarypox, adenovirus, or adeno-associated virus, can also be used for the booster, or the polyepitopic protein or a mixture of the peptides can be administered. For evaluation of vaccine efficacy, patient blood samples are obtained before immunization as well as at intervals following administration of the initial vaccine and booster doses of the vaccine. Peripheral blood mononuclear cells are isolated from fresh heparinized blood by Ficoll-Hypaque density gradient centrifugation, aliquoted in freezing media and stored frozen. Samples are assayed for CTL and HTL activity.

Analysis of the results indicates that a magnitude of sufficient response to achieve protective immunity against HIV is generated.

### Example 17

### Administration of Vaccine Compositions Using Dendritic Cells

Vaccines comprising peptide epitopes of the invention can be administered using APCs, or “professional” APCs such as DC. In this example, the peptide-pulsed DC are administered to a patient to stimulate a CTL response in vivo. In this method, dendritic cells are isolated, expanded, and pulsed with a vaccine comprising peptide CTL and HTL epitopes of the invention. The dendritic cells are infused back into the patient to elicit CTL and HTL responses in vivo. The induced CTL and HTL then destroy or facilitate destruction of the specific target cells that bear the proteins from which the epitopes in the vaccine are derived.

For example, a cocktail of epitope-bearing peptides is administered ex vivo to PBMC, or isolated DC therefrom. A pharmaceutical to facilitate harvesting of DC can be used, such as Progenipoietin™ (Monsanto, St. Louis, Mo.) or GM-CSF/IL-4. After pulsing the DC with peptides and prior to reinfusion into patients, the DC are washed to remove unbound peptides.

As appreciated clinically, and readily determined by one of skill based on clinical outcomes, the number of DC reinfused into the patient can vary (see, e.g., Nature Med. 4:328, 1998; Nature Med. 2:52, 1996 and Prostate 32:272, 1997). Although 2-50×10⁶ DC per patient are typically administered, larger number of DC, such as 10⁷ or 10⁸ can also be provided. Such cell populations typically contain between 50-90% DC.

In some embodiments, peptide-loaded PBMC are injected into patients without purification of the DC. For example, PBMC containing DC generated after treatment with an agent such as Progenipoietin™ are injected into patients without purification of the DC. The total number of PBMC that are administered often ranges from 10⁸ to 10¹⁰. Generally, the cell doses injected into patients is based on the percentage of DC in the blood of each patient, as determined, for example, by immunofluorescence analysis with specific anti-DC antibodies. Thus, for example, if Progenipoietin™ mobilizes 2% DC in the peripheral blood of a given patient, and that patient is to receive 5×10⁶ DC, then the patient will be injected with a total of 2.5×10⁸ peptide-loaded PBMC. The percent DC mobilized by an agent such as Progenipoietin™ is typically estimated to be between 2-10%, but can vary as appreciated by one of skill in the art.

Ex Vivo ACTIVATION of CTL/HTL Responses

Alternatively, ex vivo CTL or HTL responses to HIV antigens can be induced by incubating in tissue culture the patient's, or genetically compatible, CTL or HTL precursor cells together with a source of APC, such as DC, and the appropriate immunogenic peptides. After an appropriate incubation time (typically about 7-28 days), in which the precursor cells are activated and expanded into effector cells, the cells are infused back into the patient, where they will destroy or facilitate destruction of their specific target cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications and sequence listings cited herein are hereby incorporated by reference in their entirety for all purposes.

LENGTHY TABLE REFERENCED HERE
US20070054262A1-20070308-T00001
Please refer to the end of the specification for access instructions.

LENGTHY TABLE REFERENCED HERE
US20070054262A1-20070308-T00002
Please refer to the end of the specification for access instructions.

LENGTHY TABLE REFERENCED HERE
US20070054262A1-20070308-T00003
Please refer to the end of the specification for access instructions.

LENGTHY TABLE REFERENCED HERE
US20070054262A1-20070308-T00004
Please refer to the end of the specification for access instructions.

LENGTHY TABLE REFERENCED HERE
US20070054262A1-20070308-T00005
Please refer to the end of the specification for access instructions.

LENGTHY TABLE REFERENCED HERE
US20070054262A1-20070308-T00006
Please refer to the end of the specification for access instructions.

LENGTHY TABLE REFERENCED HERE
US20070054262A1-20070308-T00007
Please refer to the end of the specification for access instructions.

LENGTHY TABLE REFERENCED HERE
US20070054262A1-20070308-T00008
Please refer to the end of the specification for access instructions.

LENGTHY TABLE REFERENCED HERE
US20070054262A1-20070308-T00009
Please refer to the end of the specification for access instructions.

LENGTHY TABLE REFERENCED HERE
US20070054262A1-20070308-T00010
Please refer to the end of the specification for access instructions.

LENGTHY TABLE REFERENCED HERE
US20070054262A1-20070308-T00011
Please refer to the end of the specification for access instructions.

LENGTHY TABLE REFERENCED HERE
US20070054262A1-20070308-T00012
Please refer to the end of the specification for access instructions.

LENGTHY TABLE REFERENCED HERE
US20070054262A1-20070308-T00013
Please refer to the end of the specification for access instructions.

LENGTHY TABLE REFERENCED HERE
US20070054262A1-20070308-T00014
Please refer to the end of the specification for access instructions.

LENGTHY TABLE REFERENCED HERE
US20070054262A1-20070308-T00015
Please refer to the end of the specification for access instructions.

LENGTHY TABLE REFERENCED HERE
US20070054262A1-20070308-T00016
Please refer to the end of the specification for access instructions.

LENGTHY TABLE REFERENCED HERE
US20070054262A1-20070308-T00017
Please refer to the end of the specification for access instructions.

LENGTHY TABLE REFERENCED HERE
US20070054262A1-20070308-T00018
Please refer to the end of the specification for access instructions.

LENGTHY TABLE REFERENCED HERE
US20070054262A1-20070308-T00019
Please refer to the end of the specification for access instructions.

LENGTHY TABLE REFERENCED HERE
US20070054262A1-20070308-T00020
Please refer to the end of the specification for access instructions.

LENGTHY TABLE REFERENCED HERE
US20070054262A1-20070308-T00021
Please refer to the end of the specification for access instructions.

LENGTHY TABLE REFERENCED HERE
US20070054262A1-20070308-T00022
Please refer to the end of the specification for access instructions.

LENGTHY TABLE REFERENCED HERE
US20070054262A1-20070308-T00023
Please refer to the end of the specification for access instructions.

LENGTHY TABLE REFERENCED HERE
US20070054262A1-20070308-T00024
Please refer to the end of the specification for access instructions.

LENGTHY TABLE REFERENCED HERE
US20070054262A1-20070308-T00025
Please refer to the end of the specification for access instructions.

LENGTHY TABLE REFERENCED HERE
US20070054262A1-20070308-T00026
Please refer to the end of the specification for access instructions.

LENGTHY TABLE REFERENCED HERE
US20070054262A1-20070308-T00027
Please refer to the end of the specification for access instructions.

LENGTHY TABLE REFERENCED HERE
US20070054262A1-20070308-T00028
Please refer to the end of the specification for access instructions.

LENGTHY TABLE REFERENCED HERE
US20070054262A1-20070308-T00029
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070054262A1). An electronic copy of the table will also be available from the USPTO upon request and payment of the fee set forth in 37 CFR 1.19(b)(3).

1. A method for identifying a candidate peptide epitope which induces a HLA class I CTL response against variants of said peptide epitope, comprising
a) identifying, from a particular antigen of an infectious agent, variants of a peptide epitope 8-11 amino acids in length, each variant comprising primary anchor residues of the same HLA class I binding motif; and
b) determining whether one of said variants comprises only conserved non-anchor residues in comparison to at least one remaining variant, thereby identifying a candidate peptide epitope.

2. A method for identifying a candidate peptide epitope which induces a HLA class I CTL response against variants of said peptide epitope, comprising
a) identifying, from a particular antigen of an infectious agent, variants of a peptide epitope 8-11 amino acids in length, each variant comprising primary anchor residues of the same HLA class I binding motif;
b) determining whether each of said variants comprises conserved, semi-conserved or non-conserved non-anchor residues in comparison to each of the remaining variants; and
c) identifying a variant which comprises only conserved non-anchor residues in comparison to at least one remaining variant.

3. A method for identifying a candidate peptide epitope which induces a HLA class I CTL response against variants of said peptide epitope, comprising
a) identifying, from a particular antigen of an infectious agent, a population of variants of a peptide epitope 8-11 amino acids in length, each peptide epitope comprising primary anchor residues of the same HLA class I binding motif;
b) choosing a variant selected from the group consisting of:
i) a variant which comprises preferred primary anchor residues of said motif; and
ii) a variant which occurs with high frequency within the population of variants; and
c) determining whether the variant of (b) comprises only conserved non-anchor residues in comparison to at least one remaining variant, thereby identifying a candidate peptide epitope.

4. A method for identifying a candidate peptide epitope which induces a HLA class I CTL response against variants of said peptide epitope, comprising
a) identifying, from a particular antigen of an infectious agent, a population of variants of a peptide epitope 8-11 amino acids in length, each peptide epitope comprising primary anchor residues of the same HLA class I binding motif;
b) choosing a variant selected from the group consisting of:
i) a variant which comprises preferred primary anchor residues of said motif; and
ii) a variant which occurs with high frequency within the population of variants; and
c) determining whether the variant of (b) comprises conserved, semi-conserved or non-conserved non-anchor residues in comparison to each of the remaining variants; and
d) identifying a variant which comprises only conserved non-anchor residues in comparison to at least one remaining variant.

5. The method of claim 1, wherein (b) comprises identifying a variant which comprises only conserved non-anchor residues in comparison to at least 25%, at least 50%, at least 75%, at least 80%, at least 85%, at least 90%, at least 95%, at least 97%, or at least 99% of the remaining variants.

6. The method of claim 2, wherein (c) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

7. The method of claim 4, wherein (d) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

8-15. (canceled)

16. The method of claim 1, wherein the infectious agent is selected from the group consisting of: HIV, HBV, HCV, HPV, Plasmodium falciparum, Influenza virus, Dengue virus, Epstein-Barr virus, Mycobacterium tuberculosis, Chlamydia, Candida albicans, Cryptococcus neoformans, Coccidoides spp., Histoplasma spp, Aspergillus fumigatis, Plasmodium spp., Trypanosoma spp., Schistosoma spp., and Leishmania spp.

17-22. (canceled)

23. The method of claim 1, wherein the selected variant and the at least one remaining variant comprise different primary anchor residues of the same motif or supermotif.

24-25. (canceled)

26. The method of claim 1, wherein the variant comprises only 1-3 conserved non-anchor residues compared to at least one remaining variant.

27-30. (canceled)

31. The method of claim 3, wherein (c) comprises identifying a variant which comprises only conservative non-anchor residues in comparison to at least 25%, at least 50%, at least 75%, at least 80%, at least 85%, at least 90%, at least 95%, at least 97%, or at least 99% of the remaining variants.

32. The method of claim 2, wherein the infectious agent is selected from the group consisting of: HIV, HBV, HCV, HPV, Plasmodium falciparum, Influenza virus, Dengue virus, Epstein-Barr virus, Mycobacterium tuberculosis, Chlamydia, Candida albicans, Cryptococcus neoformans, Coccidoides spp., Histoplasma spp, Aspergillus fumigatis, Plasmodium spp., Trypanosoma spp., Schistosoma spp., and Leishmania spp.

33. The method of claim 3, wherein the infectious agent is selected from the group consisting of: HIV, HBV, HCV, HPV, Plasmodium falciparum, Influenza virus, Dengue virus, Epstein-Barr virus, Mycobacterium tuberculosis, Chlamydia, Candida albicans, Cryptococcus neoformans, Coccidoides spp., Histoplasma spp, Aspergillus fumigatis, Plasmodium spp., Trypanosoma spp., Schistosoma spp., and Leishmania spp.

34. The method of claim 4, wherein the infectious agent is selected from the group consisting of: HIV, HBV, HCV, HPV, Plasmodium falciparum, Influenza virus, Dengue virus, Epstein-Barr virus, Mycobacterium tuberculosis, Chlamydia, Candida albicans, Cryptococcus neoformans, Coccidoides spp., Histoplasma spp, Aspergillus fumigatis, Plasmodium spp., Trypanosoma spp., Schistosoma spp., and Leishmania spp.

35. The method of claim 2, wherein the selected variant and the at least one remaining variant comprise different primary anchor residues of the same motif or supermotif.

36. The method of claim 3, wherein the selected variant and the at least one remaining variant comprise different primary anchor residues of the same motif or supermotif.

37. The method of claim 4, wherein the selected variant and the at least one remaining variant comprise different primary anchor residues of the same motif or supermotif.

38. The method of claim 2, wherein the variant comprises only 1-3 conserved non-anchor residues compared to at least one remaining variant.

39. The method of claim 3, wherein the variant comprises only 1-3 conserved non-anchor residues compared to at least one remaining variant.

40. The method of claim 4, wherein the variant comprises only 1-3 conserved non-anchor residues compared to at least one remaining variant.
